SEQUENCE LISTING (27 sequences)

SEQ ID NO: 1 - DNA, 444 bp, Corynebacterium glutamicum
atgaacgtacagtttgaatcagacatggccgtccaaccaggaaacaccatggaagctaccgtcaccgacattcgtgatgccaagcgtaaaacaacccagcttgattcagtaacgccgtttaagaagaattgcccgagccgcaccttgctcgacaccatcagtgacaagtgggcggtgctgatcctgctcagcatggaaaatggtccacagcgcaatggtgaaatcaaagatcaggtccaaggaattaccccaaagatgctcacccagcgtcttggagtgttggtggaagacggactggtcactcgcacctcccacgcagttgtgccgcctcgtgtggattatcagctcaccgatctgggtgcttctgtcattgagccttgccgtgcgatgtattcctgggcagtggagaacattaagcaagtggaggcctaccgctcagcataa

SEQ ID NO: 2 - protein, 147 aa, Corynebacterium glutamicum
MNVQFESDMAVQPGNTMEATVTDIRDAKRKTTQLDSVTPFKKNCPSRTLLDTISDKWAVLILLSMENGPQRNGEIKDQVQGITPKMLTQRLGVLVEDGLVTRTSHAVVPPRVDYQLTDLGASVIEPCRAMYSWAVENIKQVEAYRSA

SEQ ID NO: 3 - DNA, 990 bp, Corynebacterium glutamicum
atggtttcaaccacaacatctcgctcaatcgctggactgtcagtgcttgtggcaacagcactaatcgctggctgtagttccgcagaggatgggacggttgactcggggagcagcacagaggtcaccacaacccaaagcaaggaaggttttcctgtcaccgtcacgtttgccccagaagcacctgtgaccattgaggatcaaccagagcgcatcgtcagtttgtccccagcgattacagaaaccttgttcgctgtcggggcaggggatcatgtcgtcgcagtggatgaatactcaaactacccagaggacgcaccgctggtgcagggtctgtctggttttactcccaatgtggagtccatcttggattacgatcctgacctggtcgtgttgatgtctgcagatgattccattttgaccggcctggatgctgcaggagtggatactttagtgatccccgcagcagagaacttggatgagacctactcccagattgaacaagtaggtcgagccaccggatttgaagatcaagcaacaacggttgttgatcagatgaaaaccgccattgatgctgcagttgccacagttcctgaagaggtaaaagagcagggcttaacctacttccacgagctgggcagtgatttgttcactgtgtcagagcaaacctacatcggtcagatttacgacatgtttggtctcacctctattgctgacggtggcgacgcttactcgcagctatccaacgaagcaatcattgcggcaaaccctgatctgattttcctcagcgatgccaaggccgaaaacctcactgcagaagatattgcggcgcgtccaggctgggacaccattgatgcagtagccaatggacgtatctacattttggacgatgatattgcttccaggtggggacctcgcgtatcccagctggtggaagaaatcgcagcgcagttgaatcagcttgcttcttctgaagctgtgccggccgctgcttaa

SEQ ID NO: 4 - protein, 329 aa, Corynebacterium glutamicum
MVSTTTSRSIAGLSVLVATALIAGCSSAEDGTVDSGSSTEVTTTQSKEGFPVTVTFAPEAPVTIEDQPERIVSLSPAITETLFAVGAGDHVVAVDEYSNYPEDAPLVQGLSGFTPNVESILDYDPDLVVLMSADDSILTGLDAAGVDTLVIPAAENLDETYSQIEQVGRATGFEDQATTVVDQMKTAIDAAVATLPEEVKEQGLTYFHELGSDLFTVSEQTYIGQIYDMFGLTSIADGGDAYSQLSNEAIIAANPDLIFLSDAKAENLTAEDIAARPGWDTIDAVANGRIYILDDDIASRWGPRVSQLVEEIAAQLNQLASSEAVPAAA

SEQ ID NO: 5 - DNA, 1041 bp, Corynebacterium glutamicum
gtgctgaagagaattttcctcaacccctgggtggctaccgcgttgtcggtagtcattttggggtttgtggtgctgttttcaggttttagcggtgttattgatttaagccccacagcagtgattagacatttgagtgggcaggacacgctcacccctcgagatcaggccatcttctttgatatccggctgcctcgaattatcgctggtgtcattgtcggagcaacgctggctatttctggtgcttcttaccaagcggtatttagaaacccgctggctgatccttatttgttgggtgtgtccgcaggttctggccttggtgtcacggcagtgattgttggcggtaccgtgctgggattttctgcaccgagcatcggcgtgattggtgcagcatttgtaggtggtgttgccgcagtacttgccacgctgatggtgagtcggggagtaggacagggatcatcaaccaccgtggttattttggcgggcgtggcggttgctgcttttgccagttccatccagacctatattcagcaacgacacatcgatacggtggcgcgcgtatatgtgtggatgttgggcaacctcaatgtcaccaactggatgtcgatcttcatcgtggctgtggtggcgggactatgcgcggccgtgatcatgtcctgcgccaggttgttagacgtgatggctgttggtgatgtggaagcccgcacattgggcgtcgatccaggcctcgtacgcattggcattgtcatcgtggcaacccttggtacagctgcagtggtatccatttccggtctcatcgggtttgtgggcatcattgttccgcacgccctgcgcctaattgttggcccggggcatcggattttactgccactgtctttcgtatggggtgccattttcctcgtgttggcagataccgcagggcgaacattgatggctcctcaggaacttcccgtgggtgtggtgacagctgcactcggcgcaccgttcttcttatttattttgcgcagaaccagcagacaacgagttccaaaaaggagtgcttaa

SEQ ID NO: 6 - protein, 346 aa, Corynebacterium glutamicum
MLKRIFLNPWVATALSVVILGFVVLFSGFSGVIDLSPTAVIRHLSGQDTLTPRDQAIFFDIRLPRIIAGVIVGATLAISGASYQAVFRNPLADPYLLGVSAGSGLGVTAVIVGGTVLGFSAPSIGVIGAAFVGGVAAVLATLMVSRGVGQGSSTTVVILAGVAVAAFASSIQTYIQQRHIDTVARVYVWMLGNLNVTNWMSIFIVAVVAGLCAAVIMSCARLLDVMAVGDVEARTLGVDPGLVRIGIVIVATLGTAAVVSISGLIGFVGIIVPHALRLIVGPGHRILLPLSFVWGAIFLVLADTAGRTLMAPQELPVGVVTAALGAPFFLFILRRTSRQRVPKRSA

SEQ ID NO: 7 - DNA, 801 bp, Corynebacterium glutamicum
gtggcgatcattgaatgcgaagacatcacggttcgccacccacatgcgcccacacctgcggtttctgggttgagcacctttgtagaacaaggggaatggcttaatatcgtaggtcccaacggctgcggaaagagtacgttgctgcacgcttttgctcaggtactgtcactggaatcgggaaggctgaaaatgggggagggggacgtcgaaaagcattttgcttttggtcttaaagctgcgaagcagcgtcgctttttcgcgcgtaccgtggccctcatgccacagaatcctactattcctgcaggtctgagcgtttttgattatgtgctgctggggcgtcatccgcacagttacgcgccggggcgtgctgatgatgagatcgtgaagcggtgcctcgctgatctgaaattggagcatttcagcgaccgcggcttagacgaattgtccggcggcgagcgtcaacgcgtcagccttgcccgcgcgctcgcccaagaaccgcgcatcgtgcttctcgacgagccgacctccgcgcttgacatcggccatgcgcaggaaacgcttgagcttatcgacgccatccggcaccgactcggcctcaccgtgatcgcggcgatgcatgacctcaccctgactgcgcaatacggcgatcgggtgctcatgatgaatggtggccgcaaagttttcgagggcactgcagccgaagtgctcaccgcgcagcggatttcggagatttatgatgccactgtgattgttgaggttattgatgggcgtcccgtggtgattccgcagcggtcgcactga

SEQ ID NO: 8 - protein, 266 aa, Corynebacterium glutamicum
MAIIECEDITVRHPHAPTPAVSGLSTFVEQGEWLNIVGPNGCGKSTLLHAFAQVLSLESGRLKMGEGDVEKHFAFGLKAAKQRRFFARTVALMPQNPTIPAGLSVFDYVLLGRHPHSYAPGRADDEIVKRCLADLKLEHFSDRGLDELSGGERQRVSLARALAQEPRIVLLDEPTSALDIGHAQETLELIDAIRHRLGLTVIAAMHDLTLTAQYGDRVLMMNGGRKVFEGTAAEVLTAQRISEIYDATVIVEVIDGRPVVIPQRSH

SEQ ID NO: 9 - DNA, 192 bp, artificial
  Promoter PSOD
gagctgccaattattccgggcttgtgacccgctacccgataaataggtcggctgaaaaatttcgttgcaatatcaacaaaaaggcctatcattgggaggtgtcgcaccaagtacttttgcgaagcgccatctgacggattttcaaaagatgtatatgctcggtgcggaaacctacgaaaggattttttaccc

SEQ ID NO: 10 - DNA, 184 bp, artificial
  Promoter PgroES
ggtcgagcggcttaaagtttggctgccatgtgaatttttagcaccctcaacagttgagtgctggcactctcgggggtagagtgccaaataggttgtttgacacacagttgttcacccgcgacgacggctgtgctggaaacccacaaccggcacacacaaaatttttctcatggagggattcatc

SEQ ID NO: 11 - DNA, 199 bp, artificial
  Promoter P EFTu
ggccgttaccctgcgaatgtccacagggtagctggtagtttgaaaatcaacgccgttgcccttaggattcagtaactggcacattttgtaatgcgctagatctgtgtgctcagtcttccaggctgcttatcacagtgaaagcaaaaccaattcgtggctgcgaaagtcgtagccaccacgaagtccaggaggacataca

SEQ ID NO: 12 - DNA, 114 bp, artificial
  Promoter lambdaPL
gtcgactcatacgttaaatctatcaccgcaagggataaatatctaacaccgtgcgtgttgactattttacctctggcggtgataatggttgcatgtactaaggaggattaatta

SEQ ID NO: 13 - DNA, 7070 bp, artificial
  Plasmid pH273
tcgagaggcctgacgtcgggcccggtaccacgcgtcatatgactagttggagaatcatgacctcagcatctgccccaagctttaaccccggcaagggtcccggctcagcagtcggaattgcccttttaggattcggaacagtcggcactgaggtgatgcgtctgatgaccgagtacggtgatgaacttgcgcaccgcattggtggcccactggaggttcgtggcattgctgtttctgatatctcaaagccacgtgaaggcgttgcacctgagctgctcactgaggacgcttttgcactcatcgagcgcgaggatgttgacatcgtcgttgaggttatcggcggcattgagtacccacgtgaggtagttctcgcagctctgaaggccggcaagtctgttgttaccgccaataaggctcttgttgcagctcactctgctgagcttgctgatgcagcggaagccgcaaacgttgacctgtacttcgaggctgctgttgcaggcgcaattccagtggttggcccactgcgtcgctccctggctggcgatcagatccagtctgtgatgggcatcgttaacggcaccaccaacttcatcttggacgccatggattccaccggcgctgactatgcagattctttggctgaggcaactcgtttgggttacgccgaagctgatccaactgcagacgtcgaaggccatgacgccgcatccaaggctgcaattttggcatccatcgctttccacacccgtgttaccgcggatgatgtgtactgcgaaggtatcagcaacatcagcgctgccgacattgaggcagcacagcaggcaggccacaccatcaagttgttggccatctgtgagaagttcaccaacaaggaaggaaagtcggctatttctgctcgcgtgcacccgactctattacctgtgtcccacccactggcgtcggtaaacaagtcctttaatgcaatctttgttgaagcagaagcagctggtcgcctgatgttctacggaaacggtgcaggtggcgcgccaaccgcgtctgctgtgcttggcgacgtcgttggtgccgcacgaaacaaggtgcacggtggccgtgctccaggtgagtccacctacgctaacctgccgatcgctgatttcggtgagaccaccactcgttaccacctcgacatggatgtggaagatcgcgtgggggttttggctgaattggctagcctgttctctgagcaaggaatcttcctgcgtacaatccgacaggaagagcgcgatgatgatgcacgtctgatcgtggtcacccactctgcgctggaatctgatctttcccgcaccgttgaactgctgaaggctaagcctgttgttaaggcaatcaacagtgtgatccgcctcgaaagggactaattttactgacatggcaattgaactgaacgtcggtcgtaaggttaccgtcacggtacctggatcttctgcaaacctcggacctggctttgacactttaggtttggcactgtcggtatacgacactgtcgaagtggaaattattccatctggcttggaagtggaagtttttggcgaaggccaaggcgaagtccctcttgatggctcccacctggtggttaaagctattcgtgctggcctgaaggcagctgacgctgaagttcctggattgcgagtggtgtgccacaacaacattccgcagtctcgtggtcttggctcctctgctgcagcggcggttgctggtgttgctgcagctaatggtttggcggatttcccgctgactcaagagcagattgttcagttgtcctctgcctttgaaggccacccagataatgctgcggcttctgtgctgggtggagcagtggtgtcgtggacaaatctgtctatcgacggcaagagccagccacagtatgctgctgtaccacttgaggtgcaggacaatattcgtgcgactgcgctggttcctaatttccacgcatccaccgaagctgtgcgccgagtccttcccactgaagtcactcacatcgatgcgcgatttaacgtgtcccgcgttgcagtgatgatcgttgcgttgcagcagcgtcctgatttgctgtgggagggtactcgtgaccgtctgcaccagccttatcgtgcagaagtgttgcctattacctctgagtgggtaaaccgcctgcgcaaccgtggctacgcggcatacctttccggtgccggcccaaccgccatggtgctgtccactgagccaattccagacaaggttttggaagatgctcgtgagtctggcattaaggtgcttgagcttgaggttgcgggaccagtcaaggttgaagttaaccaaccttaggcccaacaaggaaggcccccttcgaatcaagaagggggccttattagtgcagcaattattcgctgaacacgtgaaccttacaggtgcccggcgcgttgagtggtttgagttccagctggatgcggttgttttcaccgaggctttcttggatgaatccggcgtggatggcgcagacgaaggctgatgggcgtttgtcgttgaccacaaatgggcagctgtgtagagcgagggagtttgcttcttcggtttcggtggggtcaaagcccatttcgcggaggcggttaatgagcggggagagggcttcgtcgagttcttcggcttcggcgtggttaatgcccatgacgtgtgcccactgggttccgatggaaagtgctttggcgcggaggtcggggttgtgcattgcgtcatcgtcgacatcgccgagcatgttggccatgagttcgatcagggtgatgtattctttggcgacagcgcggttgtcggggacgcgtgtttggaagatgagggaggggcgggatcctctagacccgggatttaaatcgctagcgggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacgctagcggcgcgccggccggcccggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaaggccggccgcggccgccatcggcattttcttttgcgtttttatttgttaactgttaattgtccttgttcaaggatgctgtctttgacaacagatgttttcttgcctttgatgttcagcaggaagctcggcgcaaacgttgattgtttgtctgcgtagaatcctctgtttgtcatatagcttgtaatcacgacattgtttcctttcgcttgaggtacagcgaagtgtgagtaagtaaaggttacatcgttaggatcaagatccatttttaacacaaggccagttttgttcagcggcttgtatgggccagttaaagaattagaaacataaccaagcatgtaaatatcgttagacgtaatgccgtcaatcgtcatttttgatccgcgggagtcagtgaacaggtaccatttgccgttcattttaaagacgttcgcgcgttcaatttcatctgttactgtgttagatgcaatcagcggtttcatcacttttttcagtgtgtaatcatcgtttagctcaatcataccgagagcgccgtttgctaactcagccgtgcgttttttatcgctttgcagaagtttttgactttcttgacggaagaatgatgtgcttttgccatagtatgctttgttaaataaagattcttcgccttggtagccatcttcagttccagtgtttgcttcaaatactaagtatttgtggcctttatcttctacgtagtgaggatctctcagcgtatggttgtcgcctgagctgtagttgccttcatcgatgaactgctgtacattttgatacgtttttccgtcaccgtcaaagattgatttataatcctctacaccgttgatgttcaaagagctgtctgatgctgatacgttaacttgtgcagttgtcagtgtttgtttgccgtaatgtttaccggagaaatcagtgtagaataaacggatttttccgtcagatgtaaatgtggctgaacctgaccattcttgtgtttggtcttttaggatagaatcatttgcatcgaatttgtcgctgtctttaaagacgcggccagcgtttttccagctgtcaatagaagtttcgccgactttttgatagaacatgtaaatcgatgtgtcatccgcatttttaggatctccggctaatgcaaagacgatgtggtagccgtgatagtttgcgacagtgccgtcagcgttttgtaatggccagctgtcccaaacgtccaggccttttgcagaagagatatttttaattgtggacgaatcaaattcagaaacttgatatttttcatttttttgctgttcagggatttgcagcatatcatggcgtgtaatatgggaaatgccgtatgtttccttatatggcttttggttcgtttctttcgcaaacgcttgagttgcgcctcctgccagcagtgcggtagtaaaggttaatactgttgcttgttttgcaaactttttgatgttcatcgttcatgtctccttttttatgtactgtgttagcggtctgcttcttccagccctcctgtttgaagatggcaagttagttacgcacaataaaaaaagacctaaaatatgtaaggggtgacgccaaagtatacactttgccctttacacattttaggtcttgcctgctttatcagtaacaaacccgcgcgatttacttttcgacctcattctattagactctcgtttggattgcaactggtctattttcctcttttgtttgatagaaaatcataaaaggatttgcagactacgggcctaaagaactaaaaaatctatctgtttcttttcattctctgtattttttatagtttctgttgcatgggcataaagttgcctttttaatcacaattcagaaaatatcataatatctcatttcactaaataatagtgaacggcaggtatatgtgatgggttaaaaaggatcggcggccgctcgatttaaatc

SEQ ID NO: 14 - DNA, 7070 bp, artificial; identical to SEQ ID NO: 13
  Plasmid pH373

SEQ ID NO: 15 - DNA, 8766 bp, artificial
  Plasmid pH304
tcgagaggcctgacgtcgggcccggtaccacgcgtcatatgactagttcggacctagggatatcgtcgacatcgatgctcttctgcgttaattaacaattgggatctctcaactaatgcagcgatgcgttctttccagaatgctttcatgacagggatgctgtcttgatcaggcaggcgtctgtgctggatgccgaagctggatttattgtcgcctttggaggtgaagttgacgctcactcgagaatcatcggccaaccatttggcattgaatgttctaggttcggaggcggaggttttctcaattagtgcgggatcgagccactgcgcccgcaggtcatcgtctccgaagagcttccacactttttcgaccggcaggttaagggttttggaggcattggccgcgaacccatcgctggtcatcccgggtttgcgcatgccacgttcgtattcataaccaatcgcgatgccttgagcccaccagccactgacatcaaagttgtccacgatgtgctttgcgatgtgggtgtgagtccaagaggtggcttttacgtcgtcaagcaattttagccactcttcccacggctttccggtgccgttgaggatagcttcaggggacatgcctggtgttgagccttgcggagtggagtcagtcatgcgaccgagactagtggcgctttgggtaccgggccccccctcgaggtcgagcggcttaaagtttggctgccatgtgaatttttagcaccctcaacagttgagtgctggcactctcgggggtagagtgccaaataggttgtttgacacacagttgttcacccgcgacgacggctgtgctggaaacccacaaccggcacacacaaaatttttctcatggagggattcatcatgtcgacttcagttacttcaccagcccacaacaacgcacattcctccgaatttttggatgcgttggcaaaccatgtgttgatcggcgacggcgccatgggcacccagctccaaggctttgacctggacgtggaaaaggatttccttgatctggaggggtgtaatgagattctcaacgacacccgccctgatgtgttgaggcagattcaccgcgcctactttgaggcgggagctgacttggttgagaccaatacttttggttgcaacctgccgaacttggcggattatgacatcgctgatcgttgccgtgagcttgcctacaagggcactgcagtggctagggaagtggctgatgagatggggccgggccgaaacggcatgcggcgtttcgtggttggttccctgggacctggaacgaagcttccatcgctgggccatgcaccgtatgcagatttgcgtgggcactacaaggaagcagcgcttggcatcatcgacggtggtggcgatgcctttttgattgagactgctcaggacttgcttcaggtcaaggctgcggttcacggcgttcaagatgccatggctgaacttgatacattcttgcccattatttgccacgtcaccgtagagaccaccggcaccatgctcatgggttctgagatcggtgccgcgttgacagcgctgcagccactgggtatcgacatgattggtctgaactgcgccaccggcccagatgagatgagcgagcacctgcgttacctgtccaagcacgccgatattcctgtgtcggtgatgcctaacgcaggtcttcctgtcctgggtaaaaacggtgcagaatacccacttgaggctgaggatttggcgcaggcgctggctggattcgtctccgaatatggcctgtccatggtgggtggttgttgtggcaccacacctgagcacatccgtgcggtccgcgatgcggtggttggtgttccagagcaggaaacctccacactgaccaagatccctgcaggccctgttgagcaggcctcccgcgaggtggagaaagaggactccgtcgcgtcgctgtacacctcggtgccattgtcccaggaaaccggcatttccatgatcggtgagcgcaccaactccaacggttccaaggcattccgtgaggcaatgctgtctggcgattgggaaaagtgtgtggatattgccaagcagcaaacccgcgatggtgcacacatgctggatctttgtgtggattacgtgggacgagacggcaccgccgatatggcgaccttggcagcacttcttgctaccagctccactttgccaatcatgattgactccaccgagccagaggttattcgcacaggccttgagcacttgggtggacgaagcatcgttaactccgtcaactttgaagacggcgatggccctgagtcccgctaccagcgcatcatgaaactggtaaagcagcacggtgcggccgtggttgcgctgaccattgatgaggaaggccaggcacgtaccgctgagcacaaggtgcgcattgctaaacgactgattgacgatatcaccggcagctacggcctggatatcaaagacatcgttgtggactgcctgaccttcccgatctctactggccaggaagaaaccaggcgagatggcattgaaaccatcgaagccatccgcgagctgaagaagctctacccagaaatccacaccaccctgggtctgtccaatatttccttcggcctgaaccctgctgcacgccaggttcttaactctgtgttcctcaatgagtgcattgaggctggtctggactctgcgattgcgcacagctccaagattttgccgatgaaccgcattgatgatcgccagcgcgaagtggcgttggatatggtctatgatcgccgcaccgaggattacgatccgctgcaggaattcatgcagctgtttgagggcgtttctgctgccgatgccaaggatgctcgcgctgaacagctggccgctatgcctttgtttgagcgtttggcacagcgcatcatcgacggcgataagaatggccttgaggatgatctggaagcaggcatgaaggagaagtctcctattgcgatcatcaacgaggaccttctcaacggcatgaagaccgtgggtgagctgtttggttccggacagatgcagctgccattcgtgctgcaatcggcagaaaccatgaaaactgcggtggcctatttggaaccgttcatggaagaggaagcagaagctaccggatctgcgcaggcagagggcaagggcaaaatcgtcgtggccaccgtcaagggtgacgtgcacgatatcggcaagaacttggtggacatcattttgtccaacaacggttacgacgtggtgaacttgggcatcaagcagccactgtccgccatgttggaagcagcggaagaacacaaagcagacgtcatcggcatgtcgggacttcttgtgaagtccaccgtggtgatgaaggaaaaccttgaggagatgaacaacgccggcgcatccaattacccagtcattttgggtggcgctgcgctgacgcgtacctacgtggaaaacgatctcaacgaggtgtacaccggtgaggtgtactacgcccgtgatgctttcgagggcctgcgcctgatggatgaggtgatggcagaaaagcgtggtgaaggacttgatcccaactcaccagaagctattgagcaggcgaagaagaaggcggaacgtaaggctcgtaatgagcgttcccgcaagattgccgcggagcgtaaagctaatgcggctcccgtgattgttccggagcgttctgatgtctccaccgatactccaaccgcggcaccaccgttctggggaacccgcattgtcaagggtctgcccttggcggagttcttgggcaaccttgatgagcgcgccttgttcatggggcagtggggtctgaaatccacccgcggcaacgagggtccaagctatgaggatttggtggaaactgaaggccgaccacgcctgcgctactggctggatcgcctgaagtctgagggcattttggaccacgtggccttggtgtatggctacttcccagcggtcgcggaaggcgatgacgtggtgatcttggaatccccggatccacacgcagccgaacgcatgcgctttagcttcccacgccagcagcgcggcaggttcttgtgcatcgcggatttcattcgcccacgcgagcaagctgtcaaggacggccaagtggacgtcatgccattccagctggtcaccatgggtaatcctattgctgatttcgccaacgagttgttcgcagccaatgaataccgcgagtacttggaagttcacggcatcggcgtgcagctcaccgaagcattggccgagtactggcactcccgagtgcgcagcgaactcaagctgaacgacggtggatctgtcgctgattttgatccagaagacaagaccaagttcttcgacctggattaccgcggcgcccgcttctcctttggttacggttcttgccctgatctggaagaccgcgcaaagctggtggaattgctcgagccaggccgtatcggcgtggagttgtccgaggaactccagctgcacccagagcagtccacagacgcgtttgtgctctaccacccagaggcaaagtactttaacgtctaatctagacccgggatttaaatcgctagcgggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacgctagcggcgcgccggccggcccggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaaggccggccgcggccgccatcggcattttcttttgcgtttttatttgttaactgttaattgtccttgttcaaggatgctgtctttgacaacagatgttttcttgcctttgatgttcagcaggaagctcggcgcaaacgttgattgtttgtctgcgtagaatcctctgtttgtcatatagcttgtaatcacgacattgtttcctttcgcttgaggtacagcgaagtgtgagtaagtaaaggttacatcgttaggatcaagatccatttttaacacaaggccagttttgttcagcggcttgtatgggccagttaaagaattagaaacataaccaagcatgtaaatatcgttagacgtaatgccgtcaatcgtcatttttgatccgcgggagtcagtgaacaggtaccatttgccgttcattttaaagacgttcgcgcgttcaatttcatctgttactgtgttagatgcaatcagcggtttcatcacttttttcagtgtgtaatcatcgtttagctcaatcataccgagagcgccgtttgctaactcagccgtgcgttttttatcgctttgcagaagtttttgactttcttgacggaagaatgatgtgcttttgccatagtatgctttgttaaataaagattcttcgccttggtagccatcttcagttccagtgtttgcttcaaatactaagtatttgtggcctttatcttctacgtagtgaggatctctcagcgtatggttgtcgcctgagctgtagttgccttcatcgatgaactgctgtacattttgatacgtttttccgtcaccgtcaaagattgatttataatcctctacaccgttgatgttcaaagagctgtctgatgctgatacgttaacttgtgcagttgtcagtgtttgtttgccgtaatgtttaccggagaaatcagtgtagaataaacggatttttccgtcagatgtaaatgtggctgaacctgaccattcttgtgtttggtcttttaggatagaatcatttgcatcgaatttgtcgctgtctttaaagacgcggccagcgtttttccagctgtcaatagaagtttcgccgactttttgatagaacatgtaaatcgatgtgtcatccgcatttttaggatctccggctaatgcaaagacgatgtggtagccgtgatagtttgcgacagtgccgtcagcgttttgtaatggccagctgtcccaaacgtccaggccttttgcagaagagatatttttaattgtggacgaatcaaattcagaaacttgatatttttcatttttttgctgttcagggatttgcagcatatcatggcgtgtaatatgggaaatgccgtatgtttccttatatggcttttggttcgtttctttcgcaaacgcttgagttgcgcctcctgccagcagtgcggtagtaaaggttaatactgttgcttgttttgcaaactttttgatgttcatcgttcatgtctccttttttatgtactgtgttagcggtctgcttcttccagccctcctgtttgaagatggcaagttagttacgcacaataaaaaaagacctaaaatatgtaaggggtgacgccaaagtatacactttgccctttacacattttaggtcttgcctgctttatcagtaacaaacccgcgcgatttacttttcgacctcattctattagactctcgtttggattgcaactggtctattttcctcttttgtttgatagaaaatcataaaaggatttgcagactacgggcctaaagaactaaaaaatctatctgtttcttttcattctctgtattttttatagtttctgttgcatgggcataaagttgcctttttaatcacaattcagaaaatatcataatatctcatttcactaaataatagtgaacggcaggtatatgtgatgggttaaaaaggatcggcggccgctcgatttaaatc

SEQ ID NO: 16 - DNA, 7070 bp, artificial
  Plasmid pH399
tcgagaggcctgacgtcgggcccggtaccacgcgtcatatgactagttggagaatcatgacctcagcatctgccccaagctttaaccccggcaagggtcccggctcagcagtcggaattgcccttttaggattcggaacagtcggcactgaggtgatgcgtctgatgaccgagtacggtgatgaacttgcgcaccgcattggtggcccactggaggttcgtggcattgctgtttctgatatctcaaagccacgtgaaggcgttgcacctgagctgctcactgaggacgcttttgcactcatcgagcgcgaggatgttgacatcgtcgttgaggttatcggcggcattgagtacccacgtgaggtagttctcgcagctctgaaggccggcaagtctgttgttaccgccaataaggctcttgttgcagctcactctgctgagcttgctgatgcagcggaagccgcaaacgttgacctgtacttcgaggctgctgttgcaggcgcaattccagtggttggcccactgcgtcgctccctggctggcgatcagatccagtctgtgatgggcatcgttaacggcaccaccaacttcatcttggacgccatggattccaccggcgctgactatgcagattctttggctgaggcaactcgtttgggttacgccgaagctgatccaactgcagacgtcgaaggccatgacgccgcatccaaggctgcaattttggcatccatcgctttccacacccgtgttaccgcggatgatgtgtactgcgaaggtatcagcaacatcagcgctgccgacattgaggcagcacagcaggcaggccacaccatcaagttgttggccatctgtgagaagttcaccaacaaggaaggaaagtcggctatttctgctcgcgtgcacccgactctattacctgtgtcccacccactggcgtcggtaaacaagtcctttaatgcaatctttgttgaagcagaagcagctggtcgcctgatgttctacggaaacggtgcaggtggcgcgccaaccgcgtctgctgtgcttggcgacgtcgttggtgccgcacgaaacaaggtgcacggtggccgtgctccaggtgagtccacctacgctaacctgccgatcgctgatttcggtgagaccaccactcgttaccacctcgacatggatgtggaagatcgcgtgggggttttggctgaattggctagcctgttctctgagcaaggaatcttcctgcgtacaatccgacaggaagagcgcgatgatgatgcacgtctgatcgtggtcacccactctgcgctggaatctgatctttcccgcaccgttgaactgctgaaggctaagcctgttgttaaggcaatcaacagtgtgatccgcctcgaaagggactaattttactgacatggcaattgaactgaacgtcggtcgtaaggttaccgtcacggtacctggatcttctgcaaacctcggacctggctttgacactttaggtttggcactgtcggtatacgacactgtcgaagtggaaattattccatctggcttggaagtggaagtttttggcgaaggccaaggcgaagtccctcttgatggctcccacctggtggttaaagctattcgtgctggcctgaaggcagctgacgctgaagttcctggattgcgagtggtgtgccacaacaacattccgcagtctcgtggtcttggctccgctgctgcagcggcggttgctggtgttgctgcagctaatggtttggcggatttcccgctgactcaagagcagattgttcagttgtcctctgcctttgaaggccacccagataatgctgcggcttctgtgctgggtggagcagtggtgtcgtggacaaatctgtctatcgacggcaagagccagccacagtatgctgctgtaccacttgaggtgcaggacaatattcgtgcgactgcgctggttcctaatttccacgcatccaccgaagctgtgcgccgagtccttcccactgaagtcactcacatcgatgcgcgatttaacgtgtcccgcgttgcagtgatgatcgttgcgttgcagcagcgtcctgatttgctgtgggagggtactcgtgaccgtctgcaccagccttatcgtgcagaagtgttgcctattacctctgagtgggtaaaccgcctgcgcaaccgtggctacgcggcatacctttccggtgccggcccaaccgccatggtgctgtccactgagccaattccagacaaggttttggaagatgctcgtgagtctggcattaaggtgcttgagcttgaggttgcgggaccagtcaaggttgaagttaaccaaccttaggcccaacaaggaaggcccccttcgaatcaagaagggggccttattagtgcagcaattattcgctgaacacgtgaaccttacaggtgcccggcgcgttgagtggtttgagttccagctggatgcggttgttttcaccgaggctttcttggatgaatccggcgtggatggcgcagacgaaggctgatgggcgtttgtcgttgaccacaaatgggcagctgtgtagagcgagggagtttgcttcttcggtttcggtggggtcaaagcccatttcgcggaggcggttaatgagcggggagagggcttcgtcgagttcttcggcttcggcgtggttaatgcccatgacgtgtgcccactgggttccgatggaaagtgctttggcgcggaggtcggggttgtgcattgcgtcatcgtcgacatcgccgagcatgttggccatgagttcgatcagggtgatgtattctttggcgacagcgcggttgtcggggacgcgtgtttggaagatgagggaggggcgggatcctctagacccgggatttaaatcgctagcgggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacgctagcggcgcgccggccggcccggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaaggccggccgcggccgccatcggcattttcttttgcgtttttatttgttaactgttaattgtccttgttcaaggatgctgtctttgacaacagatgttttcttgcctttgatgttcagcaggaagctcggcgcaaacgttgattgtttgtctgcgtagaatcctctgtttgtcatatagcttgtaatcacgacattgtttcctttcgcttgaggtacagcgaagtgtgagtaagtaaaggttacatcgttaggatcaagatccatttttaacacaaggccagttttgttcagcggcttgtatgggccagttaaagaattagaaacataaccaagcatgtaaatatcgttagacgtaatgccgtcaatcgtcatttttgatccgcgggagtcagtgaacaggtaccatttgccgttcattttaaagacgttcgcgcgttcaatttcatctgttactgtgttagatgcaatcagcggtttcatcacttttttcagtgtgtaatcatcgtttagctcaatcataccgagagcgccgtttgctaactcagccgtgcgttttttatcgctttgcagaagtttttgactttcttgacggaagaatgatgtgcttttgccatagtatgctttgttaaataaagattcttcgccttggtagccatcttcagttccagtgtttgcttcaaatactaagtatttgtggcctttatcttctacgtagtgaggatctctcagcgtatggttgtcgcctgagctgtagttgccttcatcgatgaactgctgtacattttgatacgtttttccgtcaccgtcaaagattgatttataatcctctacaccgttgatgttcaaagagctgtctgatgctgatacgttaacttgtgcagttgtcagtgtttgtttgccgtaatgtttaccggagaaatcagtgtagaataaacggatttttccgtcagatgtaaatgtggctgaacctgaccattcttgtgtttggtcttttaggatagaatcatttgcatcgaatttgtcgctgtctttaaagacgcggccagcgtttttccagctgtcaatagaagtttcgccgactttttgatagaacatgtaaatcgatgtgtcatccgcatttttaggatctccggctaatgcaaagacgatgtggtagccgtgatagtttgcgacagtgccgtcagcgttttgtaatggccagctgtcccaaacgtccaggccttttgcagaagagatatttttaattgtggacgaatcaaattcagaaacttgatatttttcatttttttgctgttcagggatttgcagcatatcatggcgtgtaatatgggaaatgccgtatgtttccttatatggcttttggttcgtttctttcgcaaacgcttgagttgcgcctcctgccagcagtgcggtagtaaaggttaatactgttgcttgttttgcaaactttttgatgttcatcgttcatgtctccttttttatgtactgtgttagcggtctgcttcttccagccctcctgtttgaagatggcaagttagttacgcacaataaaaaaagacctaaaatatgtaaggggtgacgccaaagtatacactttgccctttacacattttaggtcttgcctgctttatcagtaacaaacccgcgcgatttacttttcgacctcattctattagactctcgtttggattgcaactggtctattttcctcttttgtttgatagaaaatcataaaaggatttgcagactacgggcctaaagaactaaaaaatctatctgtttcttttcattctctgtattttttatagtttctgttgcatgggcataaagttgcctttttaatcacaattcagaaaatatcataatatctcatttcactaaataatagtgaacggcaggtatatgtgatgggttaaaaaggatcggcggccgctcgatttaaatc

SEQ ID NO: 17 - DNA, 6625 bp, artificial
  Plasmid pH484
tcgagaggcctgacgtcgggcccggtaccgttgctcgctgatctttcggcttaacaactttgtattcaatcagtcgggcatagaaagaaaacgcaatgatataggaaccaactgccgccaaaaccagccacacagagttgattgtttcgccacgggagaaagcgattgctccccaacccaccgccgcgataaccccaaagacaaggagaccaacgcgggcggtcggtgacattttaggggacttcttcacgcctactggaaggtcagtagcgttgctgtacaccaaatcatcgtcattgatgttgtcagtctgttttatggtcacgatctttactgttttctcttcgggtcgtttcaaagccactatgcgtagaaacagcgggcagaaacagcgggcagaaactgtgtgcagaaatgcatgcagaaaaaggaaagttcggccagatgggtgtttctgtatgccgatgatcggatctttgacagctgggtatgcgacaaatcaccgagagttgttaattcttaacaatggaaaagtaacattgagagatgatttataccatcctgcaccatttagagtggggctagtcatacccccataaccctagctgtacgcaatcgatttcaaatcagttggaaaaagtcaagaaaattacccgagacatatgcggcttaaagtttggctgccatgtgaatttttagcaccctcaacagttgagtgctggcactctcgagggtagagtgccaaataggttgtttgacacacagttgttcacccgcgacgacggctgtgctggaaacccacaaccggcacacacaaaatttttctcatggccgttaccctgcgaatgtccacagggtagctggtagtttgaaaatcaacgccgttgcccttaggattcagtaactggcacattttgtaatgcgctagatctgtgtgctcagtcttccaggctgcttatcacagtgaaagcaaaaccaattcgtggctgcgaaagtcgtagccaccacgaagtccaggaggacatacaatgccaaagtacgacaattccaatgctgaccagtggggctttgaaacccgctccattcacgcaggccagtcagtagacgcacagaccagcgcacgaaaccttccgatctaccaatccaccgctttcgtgttcgactccgctgagcacgccaagcagcgtttcgcacttgaggatctaggccctgtttactcccgcctcaccaacccaaccgttgaggctttggaaaaccgcatcgcttccctcgaaggtggcgtccacgctgtagcgttctcctccggacaggccgcaaccaccaacgccattttgaacctggcaggagcgggcgaccacatcgtcacctccccacgcctctacggtggcaccgagactctattccttatcactcttaaccgcctgggtatcgatgtttccttcgtggaaaaccccgacgaccctgagtcctggcaggcagccgttcagccaaacaccaaagcattcttcggcgagactttcgccaacccacaggcagacgtcctggatattcctgcggtggctgaagttgcgcaccgcaacagcgttccactgatcatcgacaacaccatcgctaccgcagcgctcgtgcgcccgctcgagctcggcgcagacgttgtcgtcgcttccctcaccaagttctacaccggcaacggctccggactgggcggcgtgcttatcgacggcggaaagttcgattggactgtcgaaaaggatggaaagccagtattcccctacttcgtcactccagatgctgcttaccacggattgaagtacgcagaccttggtgcaccagccttcggcctcaaggttcgcgttggccttctacgcgacaccggctccaccctctccgcattcaacgcatgggctgcagtccagggcatcgacaccctttccctgcgcctggagcgccacaacgaaaacgccatcaaggttgcagaattcctcaacaaccacgagaaggtggaaaaggttaacttcgcaggcctgaaggattccccttggtacgcaaccaaggaaaagcttggcctgaagtacaccggctccgttctcaccttcgagatcaagggcggcaaggatgaggcttgggcatttatcgacgccctgaagctacactccaaccttgcaaacatcggcgatgttcgctccctcgttgttcacccagcaaccaccacccattcacagtccgacgaagctggcctggcacgcgcgggcgttacccagtccaccgtccgcctgtccgttggcatcgagaccattgatgatatcatcgctgacctcgaaggcggctttgctgcaatctagcactagttcggacctagggatatcgtcgacatcgatgctcttctgcgttaattaacaattgggatcctctagacccgggatttaaatcgctagcgggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacgctagcggcgcgccggccggcccggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaaggccggccgcggccgccatcggcattttcttttgcgtttttatttgttaactgttaattgtccttgttcaaggatgctgtctttgacaacagatgttttcttgcctttgatgttcagcaggaagctcggcgcaaacgttgattgtttgtctgcgtagaatcctctgtttgtcatatagcttgtaatcacgacattgtttcctttcgcttgaggtacagcgaagtgtgagtaagtaaaggttacatcgttaggatcaagatccatttttaacacaaggccagttttgttcagcggcttgtatgggccagttaaagaattagaaacataaccaagcatgtaaatatcgttagacgtaatgccgtcaatcgtcatttttgatccgcgggagtcagtgaacaggtaccatttgccgttcattttaaagacgttcgcgcgttcaatttcatctgttactgtgttagatgcaatcagcggtttcatcacttttttcagtgtgtaatcatcgtttagctcaatcataccgagagcgccgtttgctaactcagccgtgcgttttttatcgctttgcagaagtttttgactttcttgacggaagaatgatgtgcttttgccatagtatgctttgttaaataaagattcttcgccttggtagccatcttcagttccagtgtttgcttcaaatactaagtatttgtggcctttatcttctacgtagtgaggatctctcagcgtatggttgtcgcctgagctgtagttgccttcatcgatgaactgctgtacattttgatacgtttttccgtcaccgtcaaagattgatttataatcctctacaccgttgatgttcaaagagctgtctgatgctgatacgttaacttgtgcagttgtcagtgtttgtttgccgtaatgtttaccggagaaatcagtgtagaataaacggatttttccgtcagatgtaaatgtggctgaacctgaccattcttgtgtttggtcttttaggatagaatcatttgcatcgaatttgtcgctgtctttaaagacgcggccagcgtttttccagctgtcaatagaagtttcgccgactttttgatagaacatgtaaatcgatgtgtcatccgcatttttaggatctccggctaatgcaaagacgatgtggtagccgtgatagtttgcgacagtgccgtcagcgttttgtaatggccagctgtcccaaacgtccaggccttttgcagaagagatatttttaattgtggacgaatcaaattcagaaacttgatatttttcatttttttgctgttcagggatttgcagcatatcatggcgtgtaatatgggaaatgccgtatgtttccttatatggcttttggttcgtttctttcgcaaacgcttgagttgcgcctcctgccagcagtgcggtagtaaaggttaatactgttgcttgttttgcaaactttttgatgttcatcgttcatgtctccttttttatgtactgtgttagcggtctgcttcttccagccctcctgtttgaagatggcaagttagttacgcacaataaaaaaagacctaaaatatgtaaggggtgacgccaaagtatacactttgccctttacacattttaggtcttgcctgctttatcagtaacaaacccgcgcgatttacttttcgacctcattctattagactctcgtttggattgcaactggtctattttcctcttttgtttgatagaaaatcataaaaggatttgcagactacgggcctaaagaactaaaaaatctatctgtttcttttcattctctgtattttttatagtttctgttgcatgggcataaagttgcctttttaatcacaattcagaaaatatcataatatctcatttcactaaataatagtgaacggcaggtatatgtgatgggttaaaaaggatcggcggccgctcgatttaaatc

SEQ ID NO: 18 - DNA, 363 bp, artificial
  Promoter P497_P1284 = PgrESPEFTu
cggcttaaagtttggctgccatgtgaatttttagcaccctcaacagttgagtgctggcactctcgagggtagagtgccaaataggttgtttgacacacagttgttcacccgcgacgacggctgtgctggaaacccacaaccggcacacacaaaatttttctcatggccgttaccctgcgaatgtccacagggtagctggtagtttgaaaatcaacgccgttgcccttaggattcagtaactggcacattttgtaatgcgctagatctgtgtgctcagtcttccaggctgcttatcacagtgaaagcaaaaccaattcgtggctgcgaaagtcgtagccaccacgaagtccaggaggacataca

SEQ ID NO: 19 - DNA, 6350 bp, artificial
  Plasmid pH491
tcgagctcggcgcagacgttgtcgtcgcttccctcaccaagttctacaccggcaacggctccggactgggcggcgtgcttatcgacggcggaaagttcgattggactgtcgaaaaggatggaaagccagtattcccctacttcgtcactccagatgctgcttaccacggattgaagtacgcagaccttggtgcaccagccttcggcctcaaggttcgcgttggccttctacgcgacaccggctccaccctctccgcattcaacgcatgggctgcagtccagggcatcgacaccctttccctgcgcctggagcgccacaacgaaaacgccatcaaggttgcagaattcctcaacaaccacgagaaggtggaaaaggttaacttcgcaggcctgaaggattccccttggtacgcaaccaaggaaaagcttggcctgaagtacaccggctccgttctcaccttcgagatcaagggcggcaaggatgaggcttgggcatttatcgacgccctgaagctacactccaaccttgcaaacatcggcgatgttcgctccctcgttgttcacccagcaaccaccacccattcacagtccgacgaagctggcctggcacgcgcgggcgttacccagtccaccgtccgcctgtccgttggcatcgagaccattgatgatatcatcgctgacctcgaaggcggctttgctgcaatctagcactagttcggacctagggatatcgtcgagagctgccaattattccgggcttgtgacccgctacccgataaataggtcggctgaaaaatttcgttgcaatatcaacaaaaaggcctatcattgggaggtgtcgcaccaagtacttttgcgaagcgccatctgacggattttcaaaagatgtatatgctcggtgcggaaacctacgaaaggattttttacccatgcccaccctcgcgccttcaggtcaacttgaaatccaagcgatcggtgatgtctccaccgaagccggagcaatcattacaaacgctgaaatcgcctatcaccgctggggtgaataccgcgtagataaagaaggacgcagcaatgtcgttctcatcgaacacgccctcactggagattccaacgcagccgattggtgggctgacttgctcggtcccggcaaagccatcaacactgatatttactgcgtgatctgtaccaacgtcatcggtggttgcaacggttccaccggacctggctccatgcatccagatggaaatttctggggtaatcgcttccccgccacgtccattcgtgatcaggtaaacgccgaaaaacaattcctcgacgcactcggcatcaccacggtcgccgcagtacttggtggttccatgggtggtgcccgcaccctagagtgggccgcaatgtacccagaaactgttggcgcagctgctgttcttgcagtttctgcacgcgccagcgcctggcaaatcggcattcaatccgcccaaattaaggcgattgaaaacgaccaccactggcacgaaggcaactactacgaatccggctgcaacccagccaccggactcggcgccgcccgacgcatcgcccacctcacctaccgtggcgaactagaaatcgacgaacgcttcggcaccaaagcccaaaagaacgaaaacccactcggtccctaccgcaagcccgaccagcgcttcgccgtggaatcctacttggactaccaagcagacaagctagtacagcgtttcgacgccggctcctacgtcttgctcaccgacgccctcaaccgccacgacattggtcgcgaccgcggaggcctcaacaaggcactcgaatccatcaaagttccagtccttgtcgcaggcgtagataccgatattttgtacccctaccaccagcaagaacacctctccagaaacctgggaaatctactggcaatggcaaaaatcgtatcccctgtcggccacgatgctttcctcaccgaaagccgccaaatggatcgcatcgtgaggaacttcttcagcctcatctccccagacgaagacaacccttcgacctacatcgagttctacatctaacatatgactagttcggacctagggatatcgtcgacatcgatgctcttctgcgttaattaacaattgggatcctctagacccgggatttaaatcgctagcgggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacgctagcggcgcgccggccggcccggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaaggccggccgcggccgccatcggcattttcttttgcgtttttatttgttaactgttaattgtccttgttcaaggatgctgtctttgacaacagatgttttcttgcctttgatgttcagcaggaagctcggcgcaaacgttgattgtttgtctgcgtagaatcctctgtttgtcatatagcttgtaatcacgacattgtttcctttcgcttgaggtacagcgaagtgtgagtaagtaaaggttacatcgttaggatcaagatccatttttaacacaaggccagttttgttcagcggcttgtatgggccagttaaagaattagaaacataaccaagcatgtaaatatcgttagacgtaatgccgtcaatcgtcatttttgatccgcgggagtcagtgaacaggtaccatttgccgttcattttaaagacgttcgcgcgttcaatttcatctgttactgtgttagatgcaatcagcggtttcatcacttttttcagtgtgtaatcatcgtttagctcaatcataccgagagcgccgtttgctaactcagccgtgcgttttttatcgctttgcagaagtttttgactttcttgacggaagaatgatgtgcttttgccatagtatgctttgttaaataaagattcttcgccttggtagccatcttcagttccagtgtttgcttcaaatactaagtatttgtggcctttatcttctacgtagtgaggatctctcagcgtatggttgtcgcctgagctgtagttgccttcatcgatgaactgctgtacattttgatacgtttttccgtcaccgtcaaagattgatttataatcctctacaccgttgatgttcaaagagctgtctgatgctgatacgttaacttgtgcagttgtcagtgtttgtttgccgtaatgtttaccggagaaatcagtgtagaataaacggatttttccgtcagatgtaaatgtggctgaacctgaccattcttgtgtttggtcttttaggatagaatcatttgcatcgaatttgtcgctgtctttaaagacgcggccagcgtttttccagctgtcaatagaagtttcgccgactttttgatagaacatgtaaatcgatgtgtcatccgcatttttaggatctccggctaatgcaaagacgatgtggtagccgtgatagtttgcgacagtgccgtcagcgttttgtaatggccagctgtcccaaacgtccaggccttttgcagaagagatatttttaattgtggacgaatcaaattcagaaacttgatatttttcatttttttgctgttcagggatttgcagcatatcatggcgtgtaatatgggaaatgccgtatgtttccttatatggcttttggttcgtttctttcgcaaacgcttgagttgcgcctcctgccagcagtgcggtagtaaaggttaatactgttgcttgttttgcaaactttttgatgttcatcgttcatgtctccttttttatgtactgtgttagcggtctgcttcttccagccctcctgtttgaagatggcaagttagttacgcacaataaaaaaagacctaaaatatgtaaggggtgacgccaaagtatacactttgccctttacacattttaggtcttgcctgctttatcagtaacaaacccgcgcgatttacttttcgacctcattctattagactctcgtttggattgcaactggtctattttcctcttttgtttgatagaaaatcataaaaggatttgcagactacgggcctaaagaactaaaaaatctatctgtttcttttcattctctgtattttttatagtttctgttgcatgggcataaagttgcctttttaatcacaattcagaaaatatcataatatctcatttcactaaataatagtgaacggcaggtatatgtgatgggttaaaaaggatcggcggccgctcgatttaaatc

SEQ ID NO: 20 - DNA, 5477 bp, Artificial
  Plasmid pH429
tcgagctctccaatctccactgaggtacttaatccttccggggaattcgggcgcttaaatcgagaaattaggccatcaccttttaataacaatacaatgaataattggaataggtcgacacctttggagcggagccggttaaaattggcagcattcaccgaaagaaaaggagaaccacatgcttgccctaggttggattacatggatcattattggtggtctagctggttggattgcctccaagattaaaggcactgatgctcagcaaggaattttgctgaacatagtcgtcggtattatcggtggtttgttaggcggctggctgcttggaatcttcggagtggatgttgccggtggcggcttgatcttcagcttcatcacatgtctgattggtgctgtcattttgctgacgatcgtgcagttcttcactcggaagaagtaatctgctttaaatccgtagggcctgttgatatttcgatatcaacaggccttttggtcattttggggtggaaaaagcgctagacttgcctgtggattaaaactatacgaaccggtttgtctatattggtgttagacagttcgtcgtatcttgaaacagaccaacccgaaaggacgtggccgaacgtggctgctagctaatccttgatggtggacttgctggatctcgattggtccacaacatcagtcctcttgagacggctcgcgatttggctcggcagttgttgtcggctccacctgcggactactcaatttagtttcttcattttccgaaggggtatcttcgttgggggaggcgtcgataagccccttctttttagctttaacctcagcgcgacgctgctttaagcgctgcatggcggcgcggttcatttcacgttgcgtttcgcgcctcttgttcgcgatttctttgcgggcctgttttgcttcgttgatttcggcagtacgggttttggtgagttccacgtttgttgcgtgaagcgttgaggcgttccatggggtgagaatcatcagggcgcggtttttgcgtcgtgtccacaggaagatgcgcttttctttttgttttgcgcggtagatgtcgcgctgctctaggtggtgcactttgaaatcgtcggtaagtgggtatttgcgttccaaaatgaccatcatgatgattgtttggaggagcgtccacaggttgttgctgacgcgtcatatgactagttcggacctagggatatcgtcgacatcgatgctcttctgcgttaattaacaattgggatcctctagacccgggatttaaatcgctagcgggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacgctagcggcgcgccggccggcccggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaaggccggccgcggccgccatcggcattttcttttgcgtttttatttgttaactgttaattgtccttgttcaaggatgctgtctttgacaacagatgttttcttgcctttgatgttcagcaggaagctcggcgcaaacgttgattgtttgtctgcgtagaatcctctgtttgtcatatagcttgtaatcacgacattgtttcctttcgcttgaggtacagcgaagtgtgagtaagtaaaggttacatcgttaggatcaagatccatttttaacacaaggccagttttgttcagcggcttgtatgggccagttaaagaattagaaacataaccaagcatgtaaatatcgttagacgtaatgccgtcaatcgtcatttttgatccgcgggagtcagtgaacaggtaccatttgccgttcattttaaagacgttcgcgcgttcaatttcatctgttactgtgttagatgcaatcagcggtttcatcacttttttcagtgtgtaatcatcgtttagctcaatcataccgagagcgccgtttgctaactcagccgtgcgttttttatcgctttgcagaagtttttgactttcttgacggaagaatgatgtgcttttgccatagtatgctttgttaaataaagattcttcgccttggtagccatcttcagttccagtgtttgcttcaaatactaagtatttgtggcctttatcttctacgtagtgaggatctctcagcgtatggttgtcgcctgagctgtagttgccttcatcgatgaactgctgtacattttgatacgtttttccgtcaccgtcaaagattgatttataatcctctacaccgttgatgttcaaagagctgtctgatgctgatacgttaacttgtgcagttgtcagtgtttgtttgccgtaatgtttaccggagaaatcagtgtagaataaacggatttttccgtcagatgtaaatgtggctgaacctgaccattcttgtgtttggtcttttaggatagaatcatttgcatcgaatttgtcgctgtctttaaagacgcggccagcgtttttccagctgtcaatagaagtttcgccgactttttgatagaacatgtaaatcgatgtgtcatccgcatttttaggatctccggctaatgcaaagacgatgtggtagccgtgatagtttgcgacagtgccgtcagcgttttgtaatggccagctgtcccaaacgtccaggccttttgcagaagagatatttttaattgtggacgaatcaaattcagaaacttgatatttttcatttttttgctgttcagggatttgcagcatatcatggcgtgtaatatgggaaatgccgtatgtttccttatatggcttttggttcgtttctttcgcaaacgcttgagttgcgcctcctgccagcagtgcggtagtaaaggttaatactgttgcttgttttgcaaactttttgatgttcatcgttcatgtctccttttttatgtactgtgttagcggtctgcttcttccagccctcctgtttgaagatggcaagttagttacgcacaataaaaaaagacctaaaatatgtaaggggtgacgccaaagtatacactttgccctttacacattttaggtcttgcctgctttatcagtaacaaacccgcgcgatttacttttcgacctcattctattagactctcgtttggattgcaactggtctattttcctcttttgtttgatagaaaatcataaaaggatttgcagactacgggcctaaagaactaaaaaatctatctgtttcttttcattctctgtattttttatagtttctgttgcatgggcataaagttgcctttttaatcacaattcagaaaatatcataatatctcatttcactaaataatagtgaacggcaggtatatgtgatgggttaaaaaggatcggcggccgctcgatttaaatc

SEQ ID NO: 21 - DNA, 5697 bp, artificial
  Plasmid pH449
tcgaggcgtcttccggtgtcatggttgaaccgaattccagcacaatattttccggtttaaagcaatcgatcacatagtcgattttgtccaaccactgaaaacctgcaaggaccacccaatcccctgcagcatgttcagcaaccattggcagcggcggatagcgaacttcccccttttctcccgttgccattttcgcgtcactgatcaggtgactgagctttttgtagccttccggatttttacacaagactgtcaacacgccttcttgcagactcagctccgcaccataaacggtatgcattccagcttccgcggcagcttccgcaaatctcactgcaccataaaaaccatccctatccatgactgatagagcaacaagtcctaactttttggcctgcacaaccacatcagacggatccgatgcgccagtgagaaagttataactgctggtggcatgcagctcggcaaaaggaaccgacgcttccccctgcatggcagatgaaggcgcctgcgcatccggctcatgcagcaccggacgcagagattcgacctttttacctgagaggattctttccaatttggaccacgataatggcctgccgttaaagcttcccccgccattccattccataatgataggatacatttttagaacaaattttccaataagttttccacgccagccggagaaggaaatagaccaagctgtacagatcgacgcgtcctggctgagtacaacgtcggctccggcgcagacctcaccccagttggctccagcgaaatcgtgccactggcactattctggaaggaccacgactccatcgacggcattgacggcgagtccgttgccatccctaacgatccttccaaccagggccgcgccatcaacgttctcgttcaggcaggtctggtcaccctgaagaccccaggtctggtcaccccagctccagtcgatatcgacgaggcagcttccaaggtttccgtcatcccagtcgacgcagctcaggcaccaaccgcttaccaggagggtcgcccagcgatcatcaacaactccttccttgaccgcgcaggcatcgatccaaacctcgcggtcttcgaagatgatcctgagtctgaagaagcagagccatacatcaacgtcttcgtcaccaaggctgaggacaaggacgatgccaacatcgcccgcctcgttgagctgtggcacgacccagaggttctggctgcagtagaccgcgactctgagggcacctccgtcccagttgatcgtccaggagctgaccttcaggaaatccttgatcgccttgaggctgatcaggaaaacgcataatctcttttgagttctttgcatacccatgtgcagatttctttgcacaatcacagcctgaaaatcagactgtgaacttcaaacgcatatgactagttcggacctagggatatcgtcgacatcgatgctcttctgcgttaattaacaattgggatcctctagacccgggatttaaatcgctagcgggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacgctagcggcgcgccggccggcccggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaaggccggccgcggccgccatcggcattttcttttgcgtttttatttgttaactgttaattgtccttgttcaaggatgctgtctttgacaacagatgttttcttgcctttgatgttcagcaggaagctcggcgcaaacgttgattgtttgtctgcgtagaatcctctgtttgtcatatagcttgtaatcacgacattgtttcctttcgcttgaggtacagcgaagtgtgagtaagtaaaggttacatcgttaggatcaagatccatttttaacacaaggccagttttgttcagcggcttgtatgggccagttaaagaattagaaacataaccaagcatgtaaatatcgttagacgtaatgccgtcaatcgtcatttttgatccgcgggagtcagtgaacaggtaccatttgccgttcattttaaagacgttcgcgcgttcaatttcatctgttactgtgttagatgcaatcagcggtttcatcacttttttcagtgtgtaatcatcgtttagctcaatcataccgagagcgccgtttgctaactcagccgtgcgttttttatcgctttgcagaagtttttgactttcttgacggaagaatgatgtgcttttgccatagtatgctttgttaaataaagattcttcgccttggtagccatcttcagttccagtgtttgcttcaaatactaagtatttgtggcctttatcttctacgtagtgaggatctctcagcgtatggttgtcgcctgagctgtagttgccttcatcgatgaactgctgtacattttgatacgtttttccgtcaccgtcaaagattgatttataatcctctacaccgttgatgttcaaagagctgtctgatgctgatacgttaacttgtgcagttgtcagtgtttgtttgccgtaatgtttaccggagaaatcagtgtagaataaacggatttttccgtcagatgtaaatgtggctgaacctgaccattcttgtgtttggtcttttaggatagaatcatttgcatcgaatttgtcgctgtctttaaagacgcggccagcgtttttccagctgtcaatagaagtttcgccgactttttgatagaacatgtaaatcgatgtgtcatccgcatttttaggatctccggctaatgcaaagacgatgtggtagccgtgatagtttgcgacagtgccgtcagcgttttgtaatggccagctgtcccaaacgtccaggccttttgcagaagagatatttttaattgtggacgaatcaaattcagaaacttgatatttttcatttttttgctgttcagggatttgcagcatatcatggcgtgtaatatgggaaatgccgtatgtttccttatatggcttttggttcgtttctttcgcaaacgcttgagttgcgcctcctgccagcagtgcggtagtaaaggttaatactgttgcttgttttgcaaactttttgatgttcatcgttcatgtctccttttttatgtactgtgttagcggtctgcttcttccagccctcctgtttgaagatggcaagttagttacgcacaataaaaaaagacctaaaatatgtaaggggtgacgccaaagtatacactttgccctttacacattttaggtcttgcctgctttatcagtaacaaacccgcgcgatttacttttcgacctcattctattagactctcgtttggattgcaactggtctattttcctcttttgtttgatagaaaatcataaaaggatttgcagactacgggcctaaagaactaaaaaatctatctgtttcttttcattctctgtattttttatagtttctgttgcatgggcataaagttgcctttttaatcacaattcagaaaatatcataatatctcatttcactaaataatagtgaacggcaggtatatgtgatgggttaaaaaggatcggcggccgctcgatttaaatc

SEQ ID NO: 22 - DNA, 7318 bp, artificial
  Plasmid pOM427
ggccgctcgatttaaatctcgagctctggagtgcgacaggtttgatgataaaaaattagcgcaagaagacaaaaatcaccttgcgctaatgctctgttacaggtcactaataccatctaagtagttgattcatagtgactgcatatgtaagtatttccttagataacaattgattgaatgtatgcaaataaatgcatacaccataggtgtggtttaatttgatgccctttttcagggctggaatgtgtaagagcggggttatttatgctgttgtttttttgttactcgggaagggctttacctcttccgcataaacgcttccatcagcgtttatagttaaaaaaatctttcggggggatggggagtaagcttgtgttatccgctcgggcccaatccgcaagctccaccgactcgttggcgtgcgactctagataaatatcaagcagctggccgccaataacctcagtacgcatgccacgccaagcatccctcgtgcgggccaatgcctctgcactcaaaccggaatcctgcagcatgtcttctgcccacaccaatgccatatcgccagccaaaatcgagactgaaacgccaaagtgctcgggatcgccttcgaaattattggcgcggtgatcagcttccacagcccggtgaactgtgggggctccgcgccgggtatcagaagaatcgataatatcgtcatgaatcaaggcacaagcctggatgaattcgagactcgctgcggcgtcaaggacggactcaagtttttcagaagaattcttatggccttgcgccgccaggaaaccagcccacgcataaagaggacggattcgctttcctccattgagcacgaaactgcgaagatgggccacagcatctgtgacaggagcgccgatatcagcaattgttagctcttgagcatcgaggaactgcgtcaaacgatctcgcacgacctccggaaatttgtcgaggtcaaggtcatgggcatcgaaactgctcaaggagacgtccttcaatcgaatagggggatgcgggctgaattttggtggaggtgaataaatgccagaggcagtcccaacaaaacactctcatcacactaagatacccgtcgactcatacgttaaatctatcaccgcaagggataaatatctaacaccgtgcgtgttgactattttacctctggcggtgataatggttgcatgtactaaggaggattaattaatgtccctaacgaacatcccagcctcatctcaatgggcaattagcgacgttttgaagcgtccttcacccggccgagtacctttttctgtcgagtttatgccaccccgcgacgatgcagctgaagagcgtctttaccgcgcagcagaggtcttccatgacctcggtgcatcgtttgtctccgtgacttatggtgctggcggatcaacccgtgagagaacctcacgtattgctcgacgattagcgaaacaaccgttgaccactctggtgcacctgaccctggttaaccacactcgcgaagagatgaaggcaattcttcgggaatacctagagctgggattaacaaacctgttggcgcttcgaggagatccgcctggagacccattaggcgattgggtgagcaccgatggaggactgaactatgcctctgagctcatcgatcttattaagtccactcctgagttccgggaattcgacctcggtatcgcctccttccccgaagggcatttccgggcgaaaactctagaagaagacaccaaatacactctggcgaagctgcgtggaggggcagagtactccatcacgcagatgttctttgatgtggaagactacctgcgacttcgtgatcgccggatcctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttggtacgggatttaaatgatccgctagcgggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacgctagcggcgcgccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaaggccggccgcggccgccatcggcattttcttttgcgtttttatttgttaactgttaattgtccttgttcaaggatgctgtctttgacaacagatgttttcttgcctttgatgttcagcaggaagctcggcgcaaacgttgattgtttgtctgcgtagaatcctctgtttgtcatatagcttgtaatcacgacattgtttcctttcgcttgaggtacagcgaagtgtgagtaagtaaaggttacatcgttaggatcaagatccatttttaacacaaggccagttttgttcagcggcttgtatgggccagttaaagaattagaaacataaccaagcatgtaaatatcgttagacgtaatgccgtcaatcgtcatttttgatccgcgggagtcagtgaacaggtaccatttgccgttcattttaaagacgttcgcgcgttcaatttcatctgttactgtgttagatgcaatcagcggtttcatcacttttttcagtgtgtaatcatcgtttagctcaatcataccgagagcgccgtttgctaactcagccgtgcgttttttatcgctttgcagaagtttttgactttcttgacggaagaatgatgtgcttttgccatagtatgctttgttaaataaagattcttcgccttggtagccatcttcagttccagtgtttgcttcaaatactaagtatttgtggcctttatcttctacgtagtgaggatctctcagcgtatggttgtcgcctgagctgtagttgccttcatcgatgaactgctgtacattttgatacgtttttccgtcaccgtcaaagattgatttataatcctctacaccgttgatgttcaaagagctgtctgatgctgatacgttaacttgtgcagttgtcagtgtttgtttgccgtaatgtttaccggagaaatcagtgtagaataaacggatttttccgtcagatgtaaatgtggctgaacctgaccattcttgtgtttggtcttttaggatagaatcatttgcatcgaatttgtcgctgtctttaaagacgcggccagcgtttttccagctgtcaatagaagtttcgccgactttttgatagaacatgtaaatcgatgtgtcatccgcatttttaggatctccggctaatgcaaagacgatgtggtagccgtgatagtttgcgacagtgccgtcagcgttttgtaatggccagctgtcccaaacgtccaggccttttgcagaagagatatttttaattgtggacgaatcaaattcagaaacttgatatttttcatttttttgctgttcagggatttgcagcatatcatggcgtgtaatatgggaaatgccgtatgtttccttatatggcttttggttcgtttctttcgcaaacgcttgagttgcgcctcctgccagcagtgcggtagtaaaggttaatactgttgcttgttttgcaaactttttgatgttcatcgttcatgtctccttttttatgtactgtgttagcggtctgcttcttccagccctcctgtttgaagatggcaagttagttacgcacaataaaaaaagacctaaaatatgtaaggggtgacgccaaagtatacactttgccctttacacattttaggtcttgcctgctttatcagtaacaaacccgcgcgatttacttttcgacctcattctattagactctcgtttggattgcaactggtctattttcctcttttgtttgatagaaaatcataaaaggatttgcagactacgggcctaaagaactaaaaaatctatctgtttcttttcattctctgtattttttatagtttctgttgcatgggcataaagttgcctttttaatcacaattcagaaaatatcataatatctcatttcactaaataatagtgaacggcaggtatatgtgatgggttaaaaaggatcggc

SEQ ID NO: 23 - DNA, 5715 bp, artificial
  Plasmid pCLIK5A intsacB PSOD TKT
cgcgtcggcaaattagtcgaatgaagttaattaaaagttcccgaatcaatctttttaatgttttcaaaccatttgaaggtgtgctgacccaggtggacgccaacctttaaaaagcttcagacttttatttccacttcataaaaactgcctgtgacgattccgttaaagattgtgccaaatcactgcgcaaaactcgcgcggaaccagaccttgccatgctatcgcctattcacactatttgagtaatcggaaatagatgggtgtagacgcttgattggcggacggttcacagcggacgatttcaggccctcgtagctcgagagtttgaaggggtccgattcgttccgttcgtgacgctttgtgaggttttttgacgttgcaccgtattgcttgccgaacatttttcttttcctttcggtttttcgagaattttcacctacaaaagcccacgtcacagctcccagacttaagattgatcacacctttgacacatttgaaccacagttggttataaaatgggttcaacatcactatggttagaggtgttgacgggtcagattaagcaaagactactttcggggtagatcacctttgccaaatttgaaccaattaacctaagtcgtagatctgatcatcggatctaacgaaaacgaaccaaaactttggtcccggtttaacccaggaaggatagctgccaattattccgggcttgtgacccgctacccgataaataggtcggctgaaaaatttcgttgcaatatcaacaaaaaggcctatcattgggaggtgtcgcaccaagtacttttgcgaagcgccatctgacggattttcaaaagatgtatatgctcggtgcggaaacctacgaaaggattttttacccttgaccaccttgacgctgtcacctgaacttcaggcgctcactgtacgcaattacccctctgattggtccgatgtggacaccaaggctgtagacactgttcgtgtcctcgctgcagacgctgtagaaaactgtggctccggccacccaggcaccgcaatgagcctggctccccttgcatacaccttgtaccagcgggttatgaacgtagatccacaggacaccaactgggcaggccgtgaccgcttcgttctttcttgtggccactcctctttgacccagtacatccagctttacttgggtggattcggccttgagatggatgacctgaaggctctgcgcacctgggattccttgaccccaggacaccctgagtaccgccacaccaagggcgttgagatcaccactggccctcttggccagggtcttgcatctgcagttggtatggccatggctgctcgtcgtgagcgtggcctattcgacccaaccgctgctgagggcgaatccccattcgaccaccacatctacgtcattgcttctgatgggtcgacatcgatgctcttctgcgttaattaacaattgggatcctctagacccgggatttaaatgatccgctagcgggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacgctagcggcgcgccggccggcccggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaaggccggccgcggccgccatcggcattttcttttgcgtttttatttgttaactgttaattgtccttgttcaaggatgctgtctttgacaacagatgttttcttgcctttgatgttcagcaggaagctcggcgcaaacgttgattgtttgtctgcgtagaatcctctgtttgtcatatagcttgtaatcacgacattgtttcctttcgcttgaggtacagcgaagtgtgagtaagtaaaggttacatcgttaggatcaagatccatttttaacacaaggccagttttgttcagcggcttgtatgggccagttaaagaattagaaacataaccaagcatgtaaatatcgttagacgtaatgccgtcaatcgtcatttttgatccgcgggagtcagtgaacaggtaccatttgccgttcattttaaagacgttcgcgcgttcaatttcatctgttactgtgttagatgcaatcagcggtttcatcacttttttcagtgtgtaatcatcgtttagctcaatcataccgagagcgccgtttgctaactcagccgtgcgttttttatcgctttgcagaagtttttgactttcttgacggaagaatgatgtgcttttgccatagtatgctttgttaaataaagattcttcgccttggtagccatcttcagttccagtgtttgcttcaaatactaagtatttgtggcctttatcttctacgtagtgaggatctctcagcgtatggttgtcgcctgagctgtagttgccttcatcgatgaactgctgtacattttgatacgtttttccgtcaccgtcaaagattgatttataatcctctacaccgttgatgttcaaagagctgtctgatgctgatacgttaacttgtgcagttgtcagtgtttgtttgccgtaatgtttaccggagaaatcagtgtagaataaacggatttttccgtcagatgtaaatgtggctgaacctgaccattcttgtgtttggtcttttaggatagaatcatttgcatcgaatttgtcgctgtctttaaagacgcggccagcgtttttccagctgtcaatagaagtttcgccgactttttgatagaacatgtaaatcgatgtgtcatccgcatttttaggatctccggctaatgcaaagacgatgtggtagccgtgatagtttgcgacagtgccgtcagcgttttgtaatggccagctgtcccaaacgtccaggccttttgcagaagagatatttttaattgtggacgaatcaaattcagaaacttgatatttttcatttttttgctgttcagggatttgcagcatatcatggcgtgtaatatgggaaatgccgtatgtttccttatatggcttttggttcgtttctttcgcaaacgcttgagttgcgcctcctgccagcagtgcggtagtaaaggttaatactgttgcttgttttgcaaactttttgatgttcatcgttcatgtctccttttttatgtactgtgttagcggtctgcttcttccagccctcctgtttgaagatggcaagttagttacgcacaataaaaaaagacctaaaatatgtaaggggtgacgccaaagtatacactttgccctttacacattttaggtcttgcctgctttatcagtaacaaacccgcgcgatttacttttcgacctcattctattagactctcgtttggattgcaactggtctattttcctcttttgtttgatagaaaatcataaaaggatttgcagactacgggcctaaagaactaaaaaatctatctgtttcttttcattctctgtattttttatagtttctgttgcatgggcataaagttgcctttttaatcacaattcagaaaatatcataatatctcatttcactaaataatagtgaacggcaggtatatgtgatgggttaaaaaggatcggcggccgctcgatttaaatctcgagaggcctgacgtcgggcccggtacca

SEQ ID NO: 24 - DNA, 11230 bp, artificial
  Plasmid pOM474
tcgatttaaatctcgagctctggagtgcgacaggtttgatgataaaaaattagcgcaagaagacaaaaatcaccttgcgctaatgctctgttacaggtcactaataccatctaagtagttgattcatagtgactgcatatgtaagtatttccttagataacaattgattgaatgtatgcaaataaatgcatacaccataggtgtggtttaatttgatgccctttttcagggctggaatgtgtaagagcggggttatttatgctgttgtttttttgttactcgggaagggctttacctcttccgcataaacgcttccatcagcgtttatagttaaaaaaatctttcggggggatggggagtaagcttgtgttatccgctcgggcccggtaccgttgctcgctgatctttcggcttaacaactttgtattcaatcagtcgggcatagaaagaaaacgcaatgatataggaaccaactgccgccaaaaccagccacacagagttgattgtttcgccacgggagaaagcgattgctccccaacccaccgccgcgataaccccaaagacaaggagaccaacgcgggcggtcggtgacattttaggggacttcttcacgcctactggaaggtcagtagcgttgctgtacaccaaatcatcgtcattgatgttgtcagtctgttttatggtcacgatctttactgttttctcttcgggtcgtttcaaagccactatgcgtagaaacagcgggcagaaactgtgtgcagaaatgcatgcagaaaaaggaaagttcggccagatgggtgtttctgtatgccgatgatcggatctttgacagctgggtatgcgacaaatcaccgagagttgttaattcttaacaatggaaaagtaacattgagagatgatttataccatcctgcaccatttagagtggggctagtcatacccccataaccctagctgtacgcaatcgatttcaaatcagttggaaaaagtcaagaaaattacccgagacatatgcggcttaaagtttggctgccatgtgaatttttagcaccctcaacagttgagtgctggcactctcgagggtagagtgccaaataggttgtttgacacacagttgttcacccgcgacgacggctgtgctggaaacccacaaccggcacacacaaaatttttctcatggagggattcatcatgccaaagtacgacaattccaatgctgaccagtggggctttgaaacccgctccattcacgcaggccagtcagtagacgcacagaccagcgcacgaaaccttccgatctaccaatccaccgctttcgtgttcgactccgctgagcacgccaagcagcgtttcgcacttgaggatctaggccctgtttactcccgcctcaccaacccaaccgttgaggctttggaaaaccgcatcgcttccctcgaaggtggcgtccacgctgtagcgttctcctccggacaggccgcaaccaccaacgccattttgaacctggcaggagcgggcgaccacatcgtcacctccccacgcctctacggtggcaccgagactctattccttatcactcttaaccgcctgggtatcgatgtttccttcgtggaaaaccccgacgaccctgagtcctggcaggcagccgttcagccaaacaccaaagcattcttcggcgagactttcgccaacccacaggcagacgtcctggatattcctgcggtggctgaagttgcgcaccgcaacagcgttccactgatcatcgacaacaccatcgctaccgcagcgctcgtgcgcccgctcgagctcggcgcagacgttgtcgtcgcttccctcaccaagttctacaccggcaacggctccggactgggcggcgtgcttatcgacggcggaaagttcgattggactgtcgaaaaggatggaaagccagtattcccctacttcgtcactccagatgctgcttaccacggattgaagtacgcagaccttggtgcaccagccttcggcctcaaggttcgcgttggccttctacgcgacaccggctccaccctctccgcattcaacgcatgggctgcagtccagggcatcgacaccctttccctgcgcctggagcgccacaacgaaaacgccatcaaggttgcagaattcctcaacaaccacgagaaggtggaaaaggttaacttcgcaggcctgaaggattccccttggtacgcaaccaaggaaaagcttggcctgaagtacaccggctccgttctcaccttcgagatcaagggcggcaaggatgaggcttgggcatttatcgacgccctgaagctacactccaaccttgcaaacatcggcgatgttcgctccctcgttgttcacccagcaaccaccacccattcacagtccgacgaagctggcctggcacgcgcgggcgttacccagtccaccgtccgcctgtccgttggcatcgagaccattgatgatatcatcgctgacctcgaaggcggctttgctgcaatctagcactagttcggacctagggatatcgtcgagagctgccaattattccgggcttgtgacccgctacccgataaataggtcggctgaaaaatttcgttgcaatatcaacaaaaaggcctatcattgggaggtgtcgcaccaagtacttttgcgaagcgccatctgacggattttcaaaagatgtatatgctcggtgcggaaacctacgaaaggattttttacccatgcccaccctcgcgccttcaggtcaacttgaaatccaagcgatcggtgatgtctccaccgaagccggagcaatcattacaaacgctgaaatcgcctatcaccgctggggtgaataccgcgtagataaagaaggacgcagcaatgtcgttctcatcgaacacgccctcactggagattccaacgcagccgattggtgggctgacttgctcggtcccggcaaagccatcaacactgatatttactgcgtgatctgtaccaacgtcatcggtggttgcaacggttccaccggacctggctccatgcatccagatggaaatttctggggtaatcgcttccccgccacgtccattcgtgatcaggtaaacgccgaaaaacaattcctcgacgcactcggcatcaccacggtcgccgcagtacttggtggttccatgggtggtgcccgcaccctagagtgggccgcaatgtacccagaaactgttggcgcagctgctgttcttgcagtttctgcacgcgccagcgcctggcaaatcggcattcaatccgcccaaattaaggcgattgaaaacgaccaccactggcacgaaggcaactactacgaatccggctgcaacccagccaccggactcggcgccgcccgacgcatcgcccacctcacctaccgtggcgaactagaaatcgacgaacgcttcggcaccaaagcccaaaagaacgaaaacccactcggtccctaccgcaagcccgaccagcgcttcgccgtggaatcctacttggactaccaagcagacaagctagtacagcgtttcgacgccggctcctacgtcttgctcaccgacgccctcaaccgccacgacattggtcgcgaccgcggaggcctcaacaaggcactcgaatccatcaaagttccagtccttgtcgcaggcgtagataccgatattttgtacccctaccaccagcaagaacacctctccagaaacctgggaaatctactggcaatggcaaaaatcgtatcccctgtcggccacgatgctttcctcaccgaaagccgccaaatggatcgcatcgtgaggaacttcttcagcctcatctccccagacgaagacaacccttcgacctacatcgagttctacatctaacatatgactagttcggacctagggatatcgtcgactcatacgttaaatctatcaccgcaagggataaatatctaacaccgtgcgtgttgactattttacctctggcggtgataatggttgcatgtactaaggaggattaattaatgtccctaacgaacatcccagcctcatctcaatgggcaattagcgacgttttgaagcgtccttcacccggccgagtacctttttctgtcgagtttatgccaccccgcgacgatgcagctgaagagcgtctttaccgcgcagcagaggtcttccatgacctcggtgcatcgtttgtctccgtgacttatggtgctggcggatcaacccgtgagagaacctcacgtattgctcgacgattagcgaaacaaccgttgaccactctggtgcacctgaccctggttaaccacactcgcgaagagatgaaggcaattcttcgggaatacctagagctgggattaacaaacctgttggcgcttcgaggagatccgcctggagacccattaggcgattgggtgagcaccgatggaggactgaactatgcctctgagctcatcgatcttattaagtccactcctgagttccgggaattcgacctcggtatcgcctccttccccgaagggcatttccgggcgaaaactctagaagaagacaccaaatacactctggcgaagctgcgtggaggggcagagtactccatcacgcagatgttctttgatgtggaagactacctgcgacttcgtgatcgccttgtcgctgcagaccccattcatggtgcgaagccaatcattcctggcatcatgcccattacgagcctgcggtctgtgcgtcgacaggtcgaactctctggtgctcaattgccgagccaactagaagaatcacttgttcgagctgcaaacggcaatgaagaagcgaacaaagacgagatccgcaaggtgggcattgaatattccaccaatatggcagagcgactcattgccgaaggtgcggaagatctgcacttcatgacgcttaacttcacccgtgcaacccaagaagtgttgtacaaccttggcatggcgcctgcttggggagcagagcacggccaagacgcggtgcgttaaggatcctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttggtacgggatttaaatgatccgctagcgggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacgctagcggcgcgccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaaggccggccgcggccgcgcaaagtcccgcttcgtgaaaattttcgtgccgcgtgattttccgccaaaaactttaacgaacgttcgttataatggtgtcatgaccttcacgacgaagtactaaaattggcccgaatcatcagctatggatctctctgatgtcgcgctggagtccgacgcgctcgatgctgccgtcgatttaaaaacggtgatcggatttttccgagctctcgatacgacggacgcgccagcatcacgagactgggccagtgccgcgagcgacctagaaactctcgtggcggatcttgaggagctggctgacgagctgcgtgctcggccagcgccaggaggacgcacagtagtggaggatgcaatcagttgcgcctactgcggtggcctgattcctccccggcctgacccgcgaggacggcgcgcaaaatattgctcagatgcgtgtcgtgccgcagccagccgcgagcgcgccaacaaacgccacgccgaggagctggaggcggctaggtcgcaaatggcgctggaagtgcgtcccccgagcgaaattttggccatggtcgtcacagagctggaagcggcagcgagaattatcgcgatcgtggcggtgcccgcaggcatgacaaacatcgtaaatgccgcgtttcgtgtgccgtggccgcccaggacgtgtcagcgccgccaccacctgcaccgaatcggcagcagcgtcgcgcgtcgaaaaagcgcacaggcggcaagaagcgataagctgcacgaatacctgaaaaatgttgaacgccccgtgagcggtaactcacagggcgtcggctaacccccagtccaaacctgggagaaagcgctcaaaaatgactctagcggattcacgagacattgacacaccggcctggaaattttccgctgatctgttcgacacccatcccgagctcgcgctgcgatcacgtggctggacgagcgaagaccgccgcgaattcctcgctcacctgggcagagaaaatttccagggcagcaagacccgcgacttcgccagcgcttggatcaaagacccggacacggagaaacacagccgaagttataccgagttggttcaaaatcgcttgcccggtgccagtatgttgctctgacgcacgcgcagcacgcagccgtgcttgtcctggacattgatgtgccgagccaccaggccggcgggaaaatcgagcacgtaaaccccgaggtctacgcgattttggagcgctgggcacgcctggaaaaagcgccagcttggatcggcgtgaatccactgagcgggaaatgccagctcatctggctcattgatccggtgtatgccgcagcaggcatgagcagcccgaatatgcgcctgctggctgcaacgaccgaggaaatgacccgcgttttcggcgctgaccaggctttttcacataggctgagccgtggccactgcactctccgacgatcccagccgtaccgctggcatgcccagcacaatcgcgtggatcgcctagctgatcttatggaggttgctcgcatgatctcaggcacagaaaaacctaaaaaacgctatgagcaggagttttctagcggacgggcacgtatcgaagcggcaagaaaagccactgcggaagcaaaagcacttgccacgcttgaagcaagcctgccgagcgccgctgaagcgtctggagagctgatcgacggcgtccgtgtcctctggactgctccagggcgtgccgcccgtgatgagacggcttttcgccacgctttgactgtgggataccagttaaaagcggctggtgagcgcctaaaagacaccaagggtcatcgagcctacgagcgtgcctacaccgtcgctcaggcggtcggaggaggccgtgagcctgatctgccgccggactgtgaccgccagacggattggccgcgacgtgtgcgcggctacgtcgctaaaggccagccagtcgtccctgctcgtcagacagagacgcagagccagccgaggcgaaaagctctggccactatgggaagacgtggcggtaaaaaggccgcagaacgctggaaagacccaaacagtgagtacgcccgagcacagcgagaaaaactagctaagtccagtcaacgacaagctaggaaagctaaaggaaatcgcttgaccattgcaggttggtttatgactgttgagggagagactggctcgtggccgacaatcaatgaagctatgtctgaatttagcgtgtcacgtcagaccgtgaatagagcacttaaggtctgcgggcattgaacttccacgaggacgccgaaagcttcccagtaaatgtgccatctcgtaggcagaaaacggttcccccgtagggtctctctcttggcctcctttctaggtcgggctgattgctcttgaagctctctaggggggctcacaccataggcagataacgttccccaccggctcgcctcgtaagcgcacaaggactgctcccaaagatcttcaaagccactgccgcgactgccttcgcgaagccttgccccgcggaaatttcctccaccgagttcgtgcacacccctatgccaagcttctttcaccctaaattcgagagattggattcttaccgtggaaattcttcgcaaaaatcgtcccctgatcgcccttgcgacgttggcgtcggtgccgctggttgcgcttggcttgaccgacttgatcagcggccgc

SEQ ID NO: 25 - DNA, 9050 bp, artificial
  Plasmid pOM511
ggccgctcgatttaaatctcgagctctggagtgcgacaggtttgatgataaaaaattagcgcaagaagacaaaaatcaccttgcgctaatgctctgttacaggtcactaataccatctaagtagttgattcatagtgactgcatatgtaagtatttccttagataacaattgattgaatgtatgcaaataaatgcatacaccataggtgtggtttaatttgatgccctttttcagggctggaatgtgtaagagcggggttatttatgctgttgtttttttgttactcgggaagggctttacctcttccgcataaacgcttccatcagcgtttatagttaaaaaaatctttcggggggatggggagtaagcttgtgttatccgctcgggcccgctgtggcgctttgccaacgcaatggtaatcgcagcacacgcgcgagcatcttcagcagcatcgtggtggtttttcagctcaaaccctaaatgactagccaccgttggaagcttatggttttccacctgcagcttctcattgcgtgccaacgtcaacgagcagccataaatcatctctgggacatcgatccctgaggcagcacacgcgcgcgacaatgcggtgaaatcaaactgcgcattgtgagcaaccaacggcaaatccccaacgaactccaccatcttgggcacaaggtctgcaaaacgaggctgatcagcaaccatctctggggtgatgccgtgaataccaatgttgatctcattgaagaaattcaggctctcaggaggagtacacaaccatgactccgaagattcctccacaccatcgacgtacttgaccaaaccaatctggcagatggaaccccaatcatcattggccgtctctacgtcaaagcccacgaaattcagaccagcaacagtagatggagctgaaggcactactggggctccgccacccaggtgctgtggcttttcacccttcaacacagcatcaatatcagctgccagcgcagctaaatctgccgaactatttggggcaaaggcgatggatttatccacgccctccaggtttaaaaagccgtgagtaaaagcagtgggatcctgcacagatactccagagacctgtgtcagatcaaccgattccgattgtgcgctggacttagaaagagcagcaagaagaggggaataagagatggtgagggtggaatcatccaaagagatggatgcgccgtaagccgcgatcacaggtgtggtcctttcgatctgaatcaatggacaccaccctactaggtggggtggacatcatttaagcgcgccggggaattcaagaagattctggacgtgggtgtgtaacgttcaaaccaaaaatcacgacgcattaagtaacagagacttaaattaagaactccgaacaacaacttcggattgtttcatttttgaggatgaaagagcttttcaatgaacgtacagtttgaatcagacatggccgtccaaccaggaaacaccatggaagctaccgtcaccgacattcgtgatgccaagcgtaaaacaacccagcttgattcagtaacgccgtttaagaagaattgcccgagccgcaccttgctcgacaccatcagtgacaagtgggcggtgctgatcctgctcagcatggaaaatggtccacagcgcaatggtgaaatcaaagatcaggtccaaggaattaccccaaagatgctcacccagcgtcttggagtgttggtggaagacggactggtcactcgcacctcccacgcagttgtgccgcctcgtgtggattatcagctcaccgatctgggtgcttctgtcattgagccttgccgtgcgatgtattcctgggcagtggagaacattaagcaagtggaggcctaccgctcagcataagaacacttggcaaacctcacggtttccaccgtcgacgcagaattgatctctcacctaccaaacaatgcccccctgcaaaaaataaattcatataaaaaacatacagataaccatctgcggtgataaattatctctggcggtgttgacataaataccactggcggtgatactgagcacatcagcaggacgcactgaccaccatgaaggtgacgctcttaaaaattaagccctgaagaagggcatttatttgcatacattcaatcaattgttatctaaggaggattaattaatggtttcaaccacaacatctcgctcaatcgctggactgtcagtgcttgtggcaacagcactaatcgctggctgtagttccgcagaggatgggacggttgactcggggagcagcacagaggtcaccacaacccaaagcaaggaaggttttcctgtcaccgtcacgtttgccccagaagcacctgtgaccattgaggatcaaccagagcgcatcgtcagtttgtccccagcgattacagaaaccttgttcgctgtcggggcaggggatcatgtcgtcgcagtggatgaatactcaaactacccagaggacgcaccgctggtgcagggtctgtctggttttactcccaatgtggagtccatcttggattacgatcctgacctggtcgtgttgatgtctgcagatgattccattttgaccggcctggatgctgcaggagtggatactttagtgatccccgcagcagagaacttggatgagacctactcccagattgaacaagtaggtcgagccaccggatttgaagatcaagcaacaacggttgttgatcagatgaaaaccgccattgatgctgcagttgccacagttcctgaagaggtaaaagagcagggcttaacctacttccacgagctgggcagtgatttgttcactgtgtcagagcaaacctacatcggtcagatttacgacatgtttggtctcacctctattgctgacggtggcgacgcttactcgcagctatccaacgaagcaatcattgcggcaaaccctgatctgattttcctcagcgatgccaaggccgaaaacctcactgcagaagatattgcggcgcgtccaggctgggacaccattgatgcagtagccaatggacgtatctacattttggacgatgatattgcttccaggtggggacctcgcgtatcccagctggtggaagaaatcgcagcgcagttgaatcagcttgcttcttctgaagctgtgccggccgctgcttaagttttcgtgctgaagagaattttcctcaacccctgggtggctaccgcgttgtcggtagtcattttggggtttgtggtgctgttttcaggttttagcggtgttattgatttaagccccacagcagtgattagacatttgagtgggcaggacacgctcacccctcgagatcaggccatcttctttgatgtcttgaaacgtatattattcctggtaaacggggaagcggcgtcatatgcttaaacggtgcagccgcacgccttgtgcaggaaggagataaggtcattattatttcctacaaaatgatgtctgatcaagaagcggcaagccatgagccgaaagtggctgttctgaatgatcaaaacaaaattgaacaaatgctggggaacgaaccagcccgtacaattttgtaaaggatcctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttggtacgggatttaaatgatccgctagcgggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacgctagcggcgcgccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaaggccggccgcggccgccatcggcattttcttttgcgtttttatttgttaactgttaattgtccttgttcaaggatgctgtctttgacaacagatgttttcttgcctttgatgttcagcaggaagctcggcgcaaacgttgattgtttgtctgcgtagaatcctctgtttgtcatatagcttgtaatcacgacattgtttcctttcgcttgaggtacagcgaagtgtgagtaagtaaaggttacatcgttaggatcaagatccatttttaacacaaggccagttttgttcagcggcttgtatgggccagttaaagaattagaaacataaccaagcatgtaaatatcgttagacgtaatgccgtcaatcgtcatttttgatccgcgggagtcagtgaacaggtaccatttgccgttcattttaaagacgttcgcgcgttcaatttcatctgttactgtgttagatgcaatcagcggtttcatcacttttttcagtgtgtaatcatcgtttagctcaatcataccgagagcgccgtttgctaactcagccgtgcgttttttatcgctttgcagaagtttttgactttcttgacggaagaatgatgtgcttttgccatagtatgctttgttaaataaagattcttcgccttggtagccatcttcagttccagtgtttgcttcaaatactaagtatttgtggcctttatcttctacgtagtgaggatctctcagcgtatggttgtcgcctgagctgtagttgccttcatcgatgaactgctgtacattttgatacgtttttccgtcaccgtcaaagattgatttataatcctctacaccgttgatgttcaaagagctgtctgatgctgatacgttaacttgtgcagttgtcagtgtttgtttgccgtaatgtttaccggagaaatcagtgtagaataaacggatttttccgtcagatgtaaatgtggctgaacctgaccattcttgtgtttggtcttttaggatagaatcatttgcatcgaatttgtcgctgtctttaaagacgcggccagcgtttttccagctgtcaatagaagtttcgccgactttttgatagaacatgtaaatcgatgtgtcatccgcatttttaggatctccggctaatgcaaagacgatgtggtagccgtgatagtttgcgacagtgccgtcagcgttttgtaatggccagctgtcccaaacgtccaggccttttgcagaagagatatttttaattgtggacgaatcaaattcagaaacttgatatttttcatttttttgctgttcagggatttgcagcatatcatggcgtgtaatatgggaaatgccgtatgtttccttatatggcttttggttcgtttctttcgcaaacgcttgagttgcgcctcctgccagcagtgcggtagtaaaggttaatactgttgcttgttttgcaaactttttgatgttcatcgttcatgtctccttttttatgtactgtgttagcggtctgcttcttccagccctcctgtttgaagatggcaagttagttacgcacaataaaaaaagacctaaaatatgtaaggggtgacgccaaagtatacactttgccctttacacattttaggtcttgcctgctttatcagtaacaaacccgcgcgatttacttttcgacctcattctattagactctcgtttggattgcaactggtctattttcctcttttgtttgatagaaaatcataaaaggatttgcagactacgggcctaaagaactaaaaaatctatctgtttcttttcattctctgtattttttatagtttctgttgcatgggcataaagttgcctttttaatcacaattcagaaaatatcataatatctcatttcactaaataatagtgaacggcaggtatatgtgatgggttaaaaaggatcggc

SEQ ID NO: 26 - DNA, 8506 bp, artificial
  Plasmid pOM512
ggccgctcgatttaaatctcgagctctggagtgcgacaggtttgatgataaaaaattagcgcaagaagacaaaaatcaccttgcgctaatgctctgttacaggtcactaataccatctaagtagttgattcatagtgactgcatatgtaagtatttccttagataacaattgattgaatgtatgcaaataaatgcatacaccataggtgtggtttaatttgatgccctttttcagggctggaatgtgtaagagcggggttatttatgctgttgtttttttgttactcgggaagggctttacctcttccgcataaacgcttccatcagcgtttatagttaaaaaaatctttcggggggatggggagtaagcttgtgttatccgctcgggcccgctgtggcgctttgccaacgcaatggtaatcgcagcacacgcgcgagcatcttcagcagcatcgtggtggtttttcagctcaaaccctaaatgactagccaccgttggaagcttatggttttccacctgcagcttctcattgcgtgccaacgtcaacgagcagccataaatcatctctgggacatcgatccctgaggcagcacacgcgcgcgacaatgcggtgaaatcaaactgcgcattgtgagcaaccaacggcaaatccccaacgaactccaccatcttgggcacaaggtctgcaaaacgaggctgatcagcaaccatctctggggtgatgccgtgaataccaatgttgatctcattgaagaaattcaggctctcaggaggagtacacaaccatgactccgaagattcctccacaccatcgacgtacttgaccaaaccaatctggcagatggaaccccaatcatcattggccgtctctacgtcaaagcccacgaaattcagaccagcaacagtagatggagctgaaggcactactggggctccgccacccaggtgctgtggcttttcacccttcaacacagcatcaatatcagctgccagcgcagctaaatctgccgaactatttggggcaaaggcgatggatttatccacgccctccaggtttaaaaagccgtgagtaaaagcagtgggatcctgcacagatactccagagacctgtgtcagatcaaccgattccgattgtgcgctggacttagaaagagcagcaagaagaggggaataagagatggtgagggtggaatcatccaaagagatggatgcgccgtaagccgcgatcacaggtgtggtcctttcgatctgaatcaatggacaccaccctactaggtggggtggacatcatttaagcgcgccggggaattcaagaagattctggaccctaccgctcagcataagaacacttggcaaacctcacggtttccaccgtcgacgcagaattgatctctcacctaccaaacaatgcccccctgcaaaaaataaattcatataaaaaacatacagataaccatctgcggtgataaattatctctggcggtgttgacataaataccactggcggtgatactgagcacatcagcaggacgcactgaccaccatgaaggtgacgctcttaaaaattaagccctgaagaagggcatttatttgcatacattcaatcaattgttatctaaggaggattaattaatggtttcaaccacaacatctcgctcaatcgctggactgtcagtgcttgtggcaacagcactaatcgctggctgtagttccgcagaggatgggacggttgactcggggagcagcacagaggtcaccacaacccaaagcaaggaaggttttcctgtcaccgtcacgtttgccccagaagcacctgtgaccattgaggatcaaccagagcgcatcgtcagtttgtccccagcgattacagaaaccttgttcgctgtcggggcaggggatcatgtcgtcgcagtggatgaatactcaaactacccagaggacgcaccgctggtgcagggtctgtctggttttactcccaatgtggagtccatcttggattacgatcctgacctggtcgtgttgatgtctgcagatgattccattttgaccggcctggatgctgcaggagtggatactttagtgatccccgcagcagagaacttggatgagacctactcccagattgaacaagtaggtcgagccaccggatttgaagatcaagcaacaacggttgttgatcagatgaaaaccgccattgatgctgcagttgccacagttcctgaagaggtaaaagagcagggcttaacctacttccacgagctgggcagtgatttgttcactgtgtcagagcaaacctacatcggtcagatttacgacatgtttggtctcacctctattgctgacggtggcgacgcttactcgcagctatccaacgaagcaatcattgcggcaaaccctgatctgattttcctcagcgatgccaaggccgaaaacctcactgcagaagatattgcggcgcgtccaggctgggacaccattgatgcagtagccaatggacgtatctacattttggacgatgatattgcttccaggtggggacctcgcgtatcccagctggtggaagaaatcgcagcgcagttgaatcagcttgcttcttctgaagctgtgccggccgctgcttaagttttcgtgctgaagagaattttcctcaacccctgggtggctaccgcgttgtcggtagtcattttggggtttgtggtgctgttttcaggttttagcggtgttattgatttaagccccacagcagtgattagacatttgagtgggcaggacacgctcacccctcgagatcaggccatcttctttgatgtcttgaaacgtatattattcctggtaaacggggaagcggcgtcatatgcttaaacggtgcagccgcacgccttgtgcaggaaggagataaggtcattattatttcctacaaaatgatgtctgatcaagaagcggcaagccatgagccgaaagtggctgttctgaatgatcaaaacaaaattgaacaaatgctggggaacgaaccagcccgtacaattttgtaaaggatcctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttggtacgggatttaaatgatccgctagcgggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacgctagcggcgcgccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaaggccggccgcggccgccatcggcattttcttttgcgtttttatttgttaactgttaattgtccttgttcaaggatgctgtctttgacaacagatgttttcttgcctttgatgttcagcaggaagctcggcgcaaacgttgattgtttgtctgcgtagaatcctctgtttgtcatatagcttgtaatcacgacattgtttcctttcgcttgaggtacagcgaagtgtgagtaagtaaaggttacatcgttaggatcaagatccatttttaacacaaggccagttttgttcagcggcttgtatgggccagttaaagaattagaaacataaccaagcatgtaaatatcgttagacgtaatgccgtcaatcgtcatttttgatccgcgggagtcagtgaacaggtaccatttgccgttcattttaaagacgttcgcgcgttcaatttcatctgttactgtgttagatgcaatcagcggtttcatcacttttttcagtgtgtaatcatcgtttagctcaatcataccgagagcgccgtttgctaactcagccgtgcgttttttatcgctttgcagaagtttttgactttcttgacggaagaatgatgtgcttttgccatagtatgctttgttaaataaagattcttcgccttggtagccatcttcagttccagtgtttgcttcaaatactaagtatttgtggcctttatcttctacgtagtgaggatctctcagcgtatggttgtcgcctgagctgtagttgccttcatcgatgaactgctgtacattttgatacgtttttccgtcaccgtcaaagattgatttataatcctctacaccgttgatgttcaaagagctgtctgatgctgatacgttaacttgtgcagttgtcagtgtttgtttgccgtaatgtttaccggagaaatcagtgtagaataaacggatttttccgtcagatgtaaatgtggctgaacctgaccattcttgtgtttggtcttttaggatagaatcatttgcatcgaatttgtcgctgtctttaaagacgcggccagcgtttttccagctgtcaatagaagtttcgccgactttttgatagaacatgtaaatcgatgtgtcatccgcatttttaggatctccggctaatgcaaagacgatgtggtagccgtgatagtttgcgacagtgccgtcagcgttttgtaatggccagctgtcccaaacgtccaggccttttgcagaagagatatttttaattgtggacgaatcaaattcagaaacttgatatttttcatttttttgctgttcagggatttgcagcatatcatggcgtgtaatatgggaaatgccgtatgtttccttatatggcttttggttcgtttctttcgcaaacgcttgagttgcgcctcctgccagcagtgcggtagtaaaggttaatactgttgcttgttttgcaaactttttgatgttcatcgttcatgtctccttttttatgtactgtgttagcggtctgcttcttccagccctcctgtttgaagatggcaagttagttacgcacaataaaaaaagacctaaaatatgtaaggggtgacgccaaagtatacactttgccctttacacattttaggtcttgcctgctttatcagtaacaaacccgcgcgatttacttttcgacctcattctattagactctcgtttggattgcaactggtctattttcctcttttgtttgatagaaaatcataaaaggatttgcagactacgggcctaaagaactaaaaaatctatctgtttcttttcattctctgtattttttatagtttctgttgcatgggcataaagttgcctttttaatcacaattcagaaaatatcataatatctcatttcactaaataatagtgaacggcaggtatatgtgatgggttaaaaaggatcggc

SEQ ID NO: 27 - DNA, 8113 bp, artificial
  Plasmid pOM495
ggccgctcgatttaaatctcgagctctggagtgcgacaggtttgatgataaaaaattagcgcaagaagacaaaaatcaccttgcgctaatgctctgttacaggtcactaataccatctaagtagttgattcatagtgactgcatatgtaagtatttccttagataacaattgattgaatgtatgcaaataaatgcatacaccataggtgtggtttaatttgatgccctttttcagggctggaatgtgtaagagcggggttatttatgctgttgtttttttgttactcgggaagggctttacctcttccgcataaacgcttccatcagcgtttatagttaaaaaaatctttcggggggatggggagtaagcttgtgttatccgctcgggcccggtaccacgcgtcatatgactagagctgtggcgctttgccaacgcaatggtaatcgcagcacacgcgcgagcatcttcagcagcatcgtggtggtttttcagctcaaaccctaaatgactagccaccgttggaagcttatggttttccacctgcagcttctcattgcgtgccaacgtcaacgagcagccataaatcatctctgggacatcgatccctgaggcagcacacgcgcgcgacaatgcggtgaaatcaaactgcgcattgtgagcaaccaacggcaaatccccaacgaactccaccatcttgggcacaaggtctgcaaaacgaggctgatcagcaaccatctctggggtgatgccgtgaataccaatgttgatctcattgaagaaattcaggctctcaggaggagtacacaaccatgactccgaagattcctccacaccatcgacgtacttgaccaaaccaatctggcagatggaaccccaatcatcattggccgtctctacgtcaaagcccacgaaattcagaccagcaacagtagatggagctgaaggcactactggggctccgccacccaggtgctgtggcttttcacccttcaacacagcatcaatatcagctgccagcgcagctaaatctgccgaactatttggggcaaaggcgatggatttatccacgccctccaggtttaaaaagccgtgagtaaaagcagtgggatcctgcacagatactccagagacctgtgtcagatcaaccgattccgattgtgcgctggacttagaaagagcagcaagaagaggggaataagagatggtgagggtggaatcatccaaagagatggatgcgccgtaagccgcgatcacaggtgtggtcctttcgatctgaatcaatggacaccaccctactaggtggggtggacatcatttaagcgcgccggggaattcaagaagattctggaccctaccgctcagcataagaacacttggcaaacctcacggtttccaccagttaagatgttgtgagacaaatccaaacatagaagggctgtgcattctcatggtttcaaccacaacatctcgctcaatcgctggactgtcagtgcttgtggcaacagcactaatcgctggctgtagttccgcagaggatgggacggttgactcggggagcagcacagaggtcaccacaacccaaagcaaggaaggttttcctgtcaccgtcacgtttgccccagaagcacctgtgaccattgaggatcaaccagagcgcatcgtcagtttgtccccagcgattacagaaaccttgttcgctgtcggggcaggggatcatgtcgtcgcagtggatgaatactcaaactacccagaggacgcaccgctggtgcagggtctgtctggttttactcccaatgtggagtccatcttggattacgatcctgacctggtcgtgttgatgtctgcagatgattccattttgaccggcctggatgctgcaggagtggatactttagtgatccccgcagcagagaacttggatgagacctactcccagattgaacaagtaggtcgagccaccggatttgaagatcaagcaacaacggttgttgatcagatgaaaaccgccattgatgctgcagttgccacagttcctgaagaggtaaaagagcagggcttaacctacttccacgagctgggcagtgatttgttcactgtgtcagagcaaacctacatcggtcagatttacgacatgtttggtctcacctctattgctgacggtggcgacgcttactcgcagctatccaacgaagcaatcattgcggcaaaccctgatctgattttcctcagcgatgccaaggccgaaaacctcactgcagaagatattgcggcgcgtccaggctgggacaccattgatgcagtagccaatggacgtatctacattttggacgatgatattgcttccaggtggggacctcgcgtatcccagctggtggaagaaatcgcagcgcagttgaatcagcttgcttcttctgaagctgtgccggccgctgcttaagttttcgtgctgaagagaattttcctcaacccctgggtggctaccgcgttgtcggtagtcattttggggtttgtggtgctgttttcaggttttagcggtgttattgatttaagccccacagcagtgattagacatttgagtgggcaggacacgctcacccctcgaggtcgacatcgatgctcttctgcgttaattaacaattgggatcctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttggtacgggatttaaatgatccgctagcgggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacgctagcggcgcgccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaaggccggccgcggccgccatcggcattttcttttgcgtttttatttgttaactgttaattgtccttgttcaaggatgctgtctttgacaacagatgttttcttgcctttgatgttcagcaggaagctcggcgcaaacgttgattgtttgtctgcgtagaatcctctgtttgtcatatagcttgtaatcacgacattgtttcctttcgcttgaggtacagcgaagtgtgagtaagtaaaggttacatcgttaggatcaagatccatttttaacacaaggccagttttgttcagcggcttgtatgggccagttaaagaattagaaacataaccaagcatgtaaatatcgttagacgtaatgccgtcaatcgtcatttttgatccgcgggagtcagtgaacaggtaccatttgccgttcattttaaagacgttcgcgcgttcaatttcatctgttactgtgttagatgcaatcagcggtttcatcacttttttcagtgtgtaatcatcgtttagctcaatcataccgagagcgccgtttgctaactcagccgtgcgttttttatcgctttgcagaagtttttgactttcttgacggaagaatgatgtgcttttgccatagtatgctttgttaaataaagattcttcgccttggtagccatcttcagttccagtgtttgcttcaaatactaagtatttgtggcctttatcttctacgtagtgaggatctctcagcgtatggttgtcgcctgagctgtagttgccttcatcgatgaactgctgtacattttgatacgtttttccgtcaccgtcaaagattgatttataatcctctacaccgttgatgttcaaagagctgtctgatgctgatacgttaacttgtgcagttgtcagtgtttgtttgccgtaatgtttaccggagaaatcagtgtagaataaacggatttttccgtcagatgtaaatgtggctgaacctgaccattcttgtgtttggtcttttaggatagaatcatttgcatcgaatttgtcgctgtctttaaagacgcggccagcgtttttccagctgtcaatagaagtttcgccgactttttgatagaacatgtaaatcgatgtgtcatccgcatttttaggatctccggctaatgcaaagacgatgtggtagccgtgatagtttgcgacagtgccgtcagcgttttgtaatggccagctgtcccaaacgtccaggccttttgcagaagagatatttttaattgtggacgaatcaaattcagaaacttgatatttttcatttttttgctgttcagggatttgcagcatatcatggcgtgtaatatgggaaatgccgtatgtttccttatatggcttttggttcgtttctttcgcaaacgcttgagttgcgcctcctgccagcagtgcggtagtaaaggttaatactgttgcttgttttgcaaactttttgatgttcatcgttcatgtctccttttttatgtactgtgttagcggtctgcttcttccagccctcctgtttgaagatggcaagttagttacgcacaataaaaaaagacctaaaatatgtaaggggtgacgccaaagtatacactttgccctttacacattttaggtcttgcctgctttatcagtaacaaacccgcgcgatttacttttcgacctcattctattagactctcgtttggattgcaactggtctattttcctcttttgtttgatagaaaatcataaaaggatttgcagactacgggcctaaagaactaaaaaatctatctgtttcttttcattctctgtattttttatagtttctgttgcatgggcataaagttgcctttttaatcacaattcagaaaatatcataatatctcatttcactaaataatagtgaacggcaggtatatgtgatgggttaaaaaggatcggc

(19) United States
(12) Patent Application Publication
(10) Pub. No.: US 2010/0047881 A1
(43) Pub. Date: Feb. 25, 2010

(54) Microorganisms with Deregulated Vitamin B12 System

(76) Inventors: Oskar Zelder, Speyer (DE); Hartwig Schröder, Nussloch (DE); Corinna Klopprogge, Mannheim (DE); Andrea Herold, Ketsch (DE); Stefan Haefner, Speyer (DE); Thomas A. Patterson, North Attleboro, MA (US)

Correspondence Address: SMITH, GAMBRELL & RUSSELL, 1130 CONNECTICUT AVENUE, N.W., SUITE 1130, WASHINGTON, DC, 20036, US

(21) Appl. No.: 12/518,531

(22) PCT Filed: Jun. 9, 2008
(86) PCT No.: PCT/EP08/57158

§ 371 Date: Aug. 27, 2009

(30) Foreign Application Priority Data
Jun. 15, 2007 (EP) 07110382.4
May 28, 2008 (EP) 08157115.0

(51) Int. Cl. C12P 13/12; C12N 1/21

(52) U.S. Cl. 435/113; 435/252.3

(57) ABSTRACT

The present invention relates to microorganisms with improved efficiency of vitamin B12 utilization.

### FIELD OF THE INVENTION

The present invention relates to microorganisms with an increased efficiency of vitamin B12 uptake. The present invention further relates to the use of such microorganisms for obtaining fine chemicals, the biosynthesis of which requires vitamin B12 including methionine.

### BACKGROUND

Currently, the worldwide annual production of methionine is about 500,000 tons. Methionine is the first limiting amino acid in livestock of poultry feed and, due to this, mainly applied as feed supplement.

In contrast to other industrial amino acids, methionine is almost exclusively applied as a racemate of D- and L-methionine which is produced by chemical synthesis. Since animals can metabolise both stereo-isomers of methionine, direct feed of the chemically produced racemic mixture is possible (D'Mello and Lewis, Effect of Nutrition Deficiencies in Animals: Amino Acids, Rechgigl (Ed.), CRC Handbook Series in Nutrition and Food, 441-490, 1978).

However, there is still a great interest in replacing the existing chemical production by a biotechnological process producing exclusively L-methionine. This is due to the fact that at lower levels of supplementation L-methionine is a better source of sulfur amino acids than D-methionine (Katz and Baker (1975) Poult. Sci. 545: 1667-74). Moreover, the chemical process uses rather hazardous chemicals and produces substantial waste streams. All these disadvantages of chemical production could be avoided by an efficient biotechnological process.

Fermentative production of fine chemicals such as amino acids, aromatic compounds, vitamins and cofactors is today typically carried out in microorganisms such as Corynebacterium glutamicum (C. glutamicum), Escherichia coli (E. coli), Saccharomyces cerevisiae (S. cerevisiae), Schizzosaccharomycs pombe (S. pombe), Pichia pastoris (P. pastoris), Aspergillus niger, Bacillus subtilis, Ashbya gossypii or Gluconobacter oxydans.

Amino acids such as glutamate are thus produced using fermentation methods. For these purposes, certain microorganisms such as Escherichia coli (E. coli) and Corynebacterium glutamicum (C. glutamicum) have proven to be particularly suitable. The production of amino acids by fermentation also has inter alia the advantage that only L-amino acids are produced and that environmentally problematic chemicals such as solvents as they are typically used in chemical synthesis are avoided.

Some attempts in the prior art to produce fine chemicals such as amino acids, lipids, vitamins or carbohydrates in microorganisms such as E. coli and C. glutamicum have tried to achieve this goal by e.g. increasing the expression of genes involved in the biosynthetic pathways of the respective fine chemicals.

Attempts to increase production of e.g. lysine by upregulating the expression of genes being involved in the biosynthetic pathway of lysine production are e.g. described in WO 02/10209, WO 2006008097, WO2005059093 or in Cremer et al. (Appl. Environ. Microbiol, (1991), 57(6), 1746-1752).

However, strong need remains to identify further targets in metabolic pathways which can be used to beneficially influence the production of methionine or other fine chemicals in microorganisms such as C. glutamicum.

### OBJECT AND SUMMARY OF THE INVENTION

In view of this situation it is one object of the present invention to provide microorganisms and preferably Coryneform bacteria which can used to produce L-methionine. It is further object of the present invention to provide methods which can be used to product L-methionine in microorganisms and preferably in Coryneform bacteria.

These and other objectives as they will become apparent from the ensuing description are solved by the present invention as described in the independent claims. The dependent claims relate to some of the preferred embodiments of the invention.

In one embodiment, the present invention relates to a microorganism with increased efficiency of vitamin B12 uptake. In an aspect thereof the present invention relates to a microorganism with a deregulated vitamin B12 uptake system. Typically, such a vitamin B12 uptake system comprises nucleic acid sequences encoding at least one negative regulatory protein and/or at least one ABC-type transporter protein. A representative example of such a negative regulatory protein is the putative btuR2 repressor of C. glutamicum having SEQ ID No. 2. A representative example of an ABC-type transporter protein are the three sub-units that form an ABC-type transporter in C. glutamicum of SEQ ID Nos. 4, 6 and 8. In C. glutamicum the aforementioned genes are encoded by a operon that shall be referred to herein as the btu2 operon, and the three coding sequences (SEQ ID Nos. 3, 5, and 7) shall be referred to as btuF2, btuC2, and btuD2, respectively.

In a preferred embodiment, the present invention relates to a microorganism with a deregulated vitamin B12 uptake system wherein the vitamin B12 uptake system comprises nucleic acid sequences encoding at least one negative regulatory protein and/or least one ABC-type transporter protein wherein said nucleic acid sequences encoding the at least one negative regulatory protein and at least one ABC-type transporter protein are organised as an operon such that said at least one negative regulatory protein modulates expression of said at least one ABC-type transporter protein.

In a particularly preferred embodiment of the of the invention, the microorganisms comprise a deregulated vitamin B12 uptake system which comprises an operon with nucleic acid sequences encoding at least one negative regulatory protein of SEQ ID No. 2 or functional homologous or fragments thereof and at least one ABC-type transporter protein comprising sub-units of SEQ ID Nos. 4, 6 and 8 or functional homologues or fragments thereof.

In one aspect of the aforementioned embodiments, the present invention relates to microorganisms wherein the amount of said at least one negative regulatory protein is at least partially reduced by genetic alteration compared to a respective starting organism not displaying said genetic alteration. Preferably, the amount and/or activity of said at least one negative regulatory protein is completely eliminated or deleted compared to the respective starting organism.

The amount and/or activity of the above mentioned at least one negative regulatory protein may be at least partially reduced by e.g. disrupting the endogenous nucleic acid sequences of the microorganism encoding said at least one negative regulatory protein. The amount and/or activity of said at least one negative regulatory protein may also be reduced by e.g. putting the expression of said at least one negative regulatory protein under the control of a promoter that is weaker than the endogenous promoter driving expression of said protein and/or by introducing mutations in said at least one negative regulatory protein that at least partially reduce and preferably abolish the negative regulatory function of the protein on the expression of the ABC-type transporter protein.

Alternatively or in addition to this aspect of the above mentioned embodiments of the invention, the invention pertains to a microorganism wherein the amount and/or activity of said at least one ABC-type transporter protein is at least partially increased by genetic alteration compared to a respective starting organism not displaying said genetic alteration.

The amount and/or activity of the aforementioned at least one ABC-type transporter protein can be increased compared to a starting organism by increasing the copy number of nucleic acid sequences encoding said protein. The copy number of nucleic acid sequences can be increased using e.g. autonomously replicating vectors which comprise the nucleic acid sequences encoding the ABC-type transporter protein and/or by chromosomal integration of additional copies of nucleic acid sequences encoding the ABC-type transporter protein into the genome of the starting organism.

An increase of the amount and/or activity of the ABC-type transporter protein may also be achieved by increasing transcription and/or translation of a nucleic acid sequence encoding one or more subunits of such a protein. An increase of transcription may be attained by use of promoters and/or enhancer elements that ensure a stronger expression than from the endogenous promoters governing the expression of these nucleic acid sequences. An increase in translation may be achieved if the codon usage of nucleic acid sequences encoding said enzymes is optimised for the expression in the host organism or if improved binding sites and translation initiation sites for ribosomes are installed in the upstream region of the coding sequences.

The activity of an ABC-type transporter protein may also be increased compared to a starting organism by introducing mutations in the nucleic acid sequences encoding said protein that increase the activity of the protein.

In some of the preferred embodiments of the invention, the amount and/or activity of the ABC-type transporter protein is increased compared to a starting organism by combinations of the aforementioned methods.

In a preferred embodiment the microorganisms of the above mentioned aspects of the invention are selected from gram-positive microorganisms, preferably from actinobacilli and more preferably from actinomycetes.

In an even more preferred embodiment, the microorganism is selected from the genus of Corynebacterium and preferably from the species of C. glutamicum. A preferred C. glutamicum strain that can be used for the purposes of the present invention is a wild type strain such as ATCC13032 or a strain which has already been engineered for improved methionine production. Such latter strains will display genetic alterations such as those of DSM17322, M2014 or OM469 or M2543 as described hereinafter.

In a particularly preferred embodiment, the present invention concerns a microorganism wherein the microorganism is selected from the species of C. glutamicum and wherein the deregulated vitamin B12 uptake system comprises an operon with nucleic acid sequences encoding at least one negative regulatory protein of SEQ ID No. 2 or functional homologues or fragments thereof and at least one ABC-type transporter protein comprising sub-units of SEQ ID Nos. 4, 6 and 8 or functional homologues or fragments thereof.

Functional homologues or fragments of the at least one negative regulatory protein may have at least 50% sequence identity to SEQ ID No. 2. Functional homologues or fragments of the at least one ABC-type transporter protein may have at least 50% sequence identity to SEQ ID Nos. 4, 6 and 8.

A particularly preferred embodiment relates to a C. glutamicum microorganism for which expression of at least one negative regulatory protein of SEQ ID No. 1 or a functional homologue or fragment having at least 50% sequence identity to SEQ ID No. 2 is partially and preferably completely reduced by genetic alteration compared to a starting organism not displaying said genetic alterations.

Another particularly preferred embodiment relates to a C. glutamicum microorganism wherein expression of at least one ABC-type transporter protein comprising sub-units of SEQ ID Nos. 4, 6 and 8 or functional homologues or fragments thereof having at least 50% sequence identity to SEQ ID Nos. 4, 6 and 8 respectively is at least partially increased by genetic alteration such as using a strong promoter and/or an increased copy number of nucleic acid sequences encoding said sub-units compared to a starting organism not displaying said genetic alteration.

An equally particularly preferred embodiment relates to a C. glutamicum microorganism wherein expression of the at least one negative regulatory protein of SEQ ID No. 2 or a functional homologue or fragment thereof having at least 50% sequence identity to SEQ ID No. 2 is at least partially and preferably completely reduced by genetic alteration and wherein expression of the at least one ABC-type transporter protein comprising sub-units of SEQ ID Nos. 4, 6 and 8 or functional homologues or fragments thereof having at least 50% sequence identity to SEQ ID Nos. 4, 6 and 8 is at least partially increased by genetic alteration such as using a strong promoter and/or an increased copy number of nucleic acid sequences encoding said sub-units compared to a starting organism not displaying said genetic alterations.

The present invention also pertains to the use of the afore-described microorganisms and particularly preferred embodiments thereof for obtaining fine chemicals, the biological synthesis of which requires vitamin B12. Such fine chemicals include methionine, S-adenosyl methionine, and methionine sulfoxide.

The present invention also pertains to a method of obtaining a fine chemical, the biological synthesis of which requires vitamin B12, comprising the steps of cultivating a microorganism as described hereinafter and obtaining said fine chemical. The fine chemical may again be selected from the aforementioned group.

### FIGURES

FIG. 1 depicts the impact of btuR2 deletion on vitamin B12 uptake.

### DETAILED DESCRIPTION OF THE INVENTION

The inventors of the present invention have surprisingly found that C. glutamicum comprises a vitamin B12 uptake system which consists of an operon comprising nucleic acid sequences for a negative regulatory protein and an ABC-type transporter protein wherein the negative regulatory protein seems to down-regulate expression of the ABC-type transporter protein.

Such a system, which is generically designated btu (an abbreviation of B 12 uptake) hereinafter, seems to ensure transport of vitamin B12 into the cell. By deregulating the system it is possible to provide microorganisms which more efficiently use vitamin B12. As a consequence one can grow microorganisms that are commonly used for production of fine chemicals such as methionine, S-adenosyl methionine, and methionine sulfoxide that require vitamin B12 for their synthesis in a medium that comprises less vitamin B12 than one would ordinarily use.

Thus, the inventors of the present invention have found that if the mutative negative regulatory protein designated hereinafter btuR2 is deleted in C. glutamicum, cells can be grown in media containing significantly less vitamin B12 than usual. In view of these findings it seems justified that the same effect can be achieved if one alternatively or in addition over-expresses the sequences encoding for the sub-units of the ABC-type transporter protein that also forms part of the btu2 operon and which are designated butF2, butC2 and butD2. Further, the findings disclosed herein can be used to identify homologous sequences of comparable function in other gram-positive microorganisms and particularly for microorganisms selected from the group of actinobacilli and more preferably from the group of actinomycetes that will display comparable vitamin B12 uptake systems.

Before specific aspects and some of the preferred embodiments of the invention are described in more detail, the following definitions are provided which shall have the indicated meaning throughout the description of the invention, unless explicitly indicated otherwise by the respective context.

The present invention as illustratively described in the following may suitably be practiced in the absence of any element or elements, limitation or limitations, not specifically disclosed herein.

The present invention will be described with respect to particular embodiments but the invention is not limited thereto but only by the claims.

Where the term “comprising” is used in the present description and claims, it does not exclude other elements. For the purposes of the present invention, the term “consisting of” is considered to be a preferred embodiment of the term “comprising of”. If hereinafter a group is defined to comprise at least a certain number of embodiments, this is also to be understood to disclose a group which preferably consists only of these embodiments.

Where an indefinite or definite article is used when referring to a singular noun, e.g. a “an” or “the”, this includes a plural of that noun unless something else is specifically stated. The terms “about” or “approximately” in the context of the present invention, denote an interval of accuracy that the person skilled in the art will understand to still ensure the technical effect of the feature in question. The term typically indicates deviation from the indicated numerical value of ±10%, and preferably of ±5%.

The term “microorganisms” in the context of the present invention refers to any prokaryotic type of microorganisms. The term thus comprises gram-negative microorganisms such as E. coli and gram-positive microorganisms such as C. glutamicum. Preferably, microorganisms are selected from the group of gram-positive microorganisms or more preferably from the group of actinobacilli and even more preferably of the group actinomycetes. The genes Coryneform bacteria form a particularly preferred sub-set of microorganisms in accordance with the invention.

Coryneform bacteria comprise species such as Corynebacterium glutamicum, Corynebacterium jeikeum, Corynebacterium acetoglutamicum, Corynebacterium acetoacidophilum, Corynebacterium thermoaminogenes, Corynebacterium melassecola and Corynebacterium efficiens. A preferred species is C. glutamicum.

In preferred embodiments of the invention Coryneform bacteria may be derived from the group of strains comprising C. glutamicum ATCC13032, C. glutamicum KFCC10065, C. glutamicum ATCC21608C. acetoglutamicum ATCC15806, C. acetoacidophilum ATCC13870, C. thermoaminogenes FERMBP-1539, C. melassecola ATCC17965, C. efficiens DSM 44547 and C. efficiens DSM 44549, as well as strains that are derived thereof by e.g. classical mutagenesis and selection or by directed mutagenesis.

Other particularly preferred strains of C. glutamicum may be selected from the group comprising ATCC13058, ATCC13059, ATCC13060, ATCC21492, ATCC21513, ATCC21526, ATCC21543, ATCC13287, ATCC21851, ATCC21253, ATCC21514, ATCC21516, ATCC21299, ATCC21300, ATCC39684, ATCC21488, ATCC21649, ATCC21650, ATCC19223, ATCC13869, ATCC21157, ATCC21158, ATCC21159, ATCC21355, ATCC31808, ATCC21674, ATCC21562, ATCC21563, ATCC21564, ATCC21565, ATCC21566, ATCC21567, ATCC21568, ATCC21569, ATCC21570, ATCC21571, ATCC21572, ATCC21573, ATCC21579, ATCC19049, ATCC19050, ATCC19051, ATCC19052, ATCC19053, ATCC19054, ATCC19055, ATCC19056, ATCC19057, ATCC19058, ATCC19059, ATCC19060, ATCC19185, ATCC13286, ATCC21515, ATCC21527, ATCC21544, ATCC21492, NRRL B8183, NRRL W8182, B12NRRLB12416, NRRLB12417, NRRLB12418 and NRRLB11476.

The abbreviation KFCC stands for Korean Federation of Culture Collection, ATCC stands for American-Type Strain Culture Collection and the abbreviation DSM stands for Deutsche Sammlung von Mikroorganismen. The abbreviation NRRL stands for ARS cultures collection Northern Regional Research Laboratory, Peorea, EL, USA.

For the purposes of the present invention, a preferred wild-type strain is C. glutamicum ATCC13032.

Particularly preferred are microorganisms of Corynebacterium glutamicum that are already capable of producing methionine. Therefore, strains that display genetic alterations having a similar effect such as DSM17322; M2014, OM469 or M2543 being described below are particularly preferred.

In this specification, particular proteins may be referred to by the name of the gene that encodes said protein. For example, “btuR2” may refer to either the gene btuR2 or the protein encoded by the gene btuR2.

The term “starting organism” within the context of the present invention refers to a microorganism and preferably to Coryneform bacterium and particularly preferably to a C. glutamicum microorganism which is used for genetic modification to decrease the amount and/or activity of the above mentioned negative regulatory protein and/or to increase the amount and/or activity of the aforementioned ABC-type transporter proteins.

The terms “genetic modification” and “genetic alteration” as well as their grammatical variations within the meaning of the present invention are intended to mean that a microorganism has been modified by means of gene technology to express an altered amount of one or more proteins which can be naturally present in the respective microorganism, one or more proteins which are not naturally present in the respective microorganism, or one or more proteins with an altered activity in comparison to the proteins of the respective non-modified microorganism. A non-modified microorganism is considered to be a “starting organism”, the genetic alteration of which results in a microorganism in accordance with the present invention.

The starting organism may thus be a wild-type C. glutamicum strain such as ATCC13032.

However, the starting organism may preferably also be e.g. a C. glutamicum strain which has already been optimized for production of methionine.

Such a methionine-producing starting organism can e.g. be derived from a wild type Coryneform bacterium and preferably from a wild type C. glutamicum bacterium which contains genetic alterations in at least one of the following genes: ask^{fbr}, hom^{fbr} and metH wherein the genetic alterations lead to overexpression of any of these genes, thereby resulting in increased production of methionine relative to methionine produced in the absence of the genetic alterations. In a preferred embodiment, such a methionine producing starter organism will contain genetic alterations simultaneously in ask^{fbr}, hom^{fbr} and metH thereby resulting in increased production of methionine relative to methionine produced in the absence of the genetic alterations.

In these starting organisms, the endogenous copies of ask and hom are typically changed to feedback resistant alleles which are no longer subject to feedback inhibition by lysine threonine, methionine or by a combination of these amino acids. This can be either done by mutation and selection or by defined genetic replacements of the genes by with mutated alleles which code for proteins with reduced or diminished feedback inhibition. A C. glutamicum strain which includes these genetic alterations is e.g. C. glutamicum DSM17322. The person skilled in the art will be aware that alternative genetic alterations to those being described below for generation of C. glutamicum DSM17322 can be used to also achieve overexpression of ask ask^{fbr}, hom^{fbr} and metH.

For the purposes of the present invention, ask^{fbr} denotes a feedback resistant aspartate kinase. Hom^{fbr} denotes a feedback resistant homoserine dehydrogenase. MetH denotes a Vitamin B12-dependent methionine synthase.

In another preferred embodiment, a methionine-producing starting organism can be derived from a wild type Coryneform bacterium and preferably from wild type C. glutamicum bacterium which contains genetic alterations in at least one of the following genes: ask^{fbr}, hom^{fbr}, metH, metA (also referred to as metX), metY (also referred to as metZ), and hsk^{mutated} and met^{F} and tkt wherein the genetic alterations lead to over-expression of any of these genes, thereby resulting in increased production of methionine relative to methionine produced in the absence of the genetic alterations. In a preferred embodiment, such a methionine producing starter organism will contain genetic alterations simultaneously in ask^{fbr}, hum^{fbr}, metH, metA (also referred to as metX), metY (also referred to as metZ), and hsk^{mutated} and metF thereby resulting in increased production of methionine relative to methionine produced in the absence of the genetic alterations.

In these starting organisms, the endogenous copies of ask, hom and hsk are typically replaced by ask^{fbr}, hom^{fbr} and hsk^{mutated} as described above for ask^{fbr} and hom^{fbr}. A C. glutamicum strain which includes these genetic alterations is e.g. C. glutamicum M2014. The person skilled in the art will be aware that alternative genetic alterations to those being described below specifically for generation of C. glutamicum M2014 can be used to also achieve overexpression of ask^{fbr}, hom^{fbr}, metH, metA (also referred to as metX), metY (also referred to as metZ), and hsk^{mutated}.

For the purposes of the present invention, metA denotes a homoserine succinyltransferase e.g. from E. coli. MetY denotes a O-Acetylhomoserine sulfhydrylase. Hsk^{mutated} denotes a homoserine kinase which has been mutated to reduce enzymatic activity. This may be achieved by exchanging threonine with serine or alanine at a position corresponding to T190 of hsk of SEQ ID No. 19. Alternatively or additionally one may replace the ATG start codon with a TTG start codon. Such mutations lead to a reduction in enzymatic activity of the resulting hsk protein compared the non-mutated hsk gene.

In another preferred embodiment, a methionine-producing starting organism can be derived from a wild type Coryneform bacterium and preferably from a wild type C. glutamicum bacterium which contains genetic alterations in at least one of the following genes: ask^{fbr}, hom^{fbr}, metH, metA (also referred to as metX), metY (also referred to as metZ), hsk^{mutated} and metF wherein the genetic alterations lead to overexpression of any of these genes, in combination with genetic alterations in at least one of the following genes: mcbR and metQ wherein the genetic alterations decrease expression of any of these genes where the combination results in increased methionine production by the microorganism relative to methionine production in absence of the combination. In a preferred embodiment, such a methionine producing starter organism will contain genetic alterations simultaneously in ask^{fbr}, hom^{fbr}, metH, metA (also referred to as metX), metY (also referred to as metZ), hsk^{mutated} and metF wherein the genetic alterations lead to overexpression of any of these genes, in combination with genetic alterations in mcbR and metQ wherein the genetic alterations decrease expression of any of these genes where the combination results in increased methionine production by the microorganism relative to methionine production in absence of the combination.

In these starting organisms, the endogenous copies of ask, hom and hsk are typically replaced as described above while the endogenous copies of mcbR and metQ are typically functionally disrupted or deleted. A C. glutamicum strain which includes these genetic alterations is e.g. C. glutamicum OM469. The person skilled in the art will be aware that alternative genetic alterations to those being described below specifically for generation of C. glutamicum OM469 can be used to also achieve overexpression of ask^{fbr}, hom^{fbr}, metH, metA (also referred to as metX), metY (also referred to as metZ), hsk^{mutated} and metF and reduced expression of mcbR and metQ.

For the purposes of the present invention, metF denotes a N5,10-methylene-tetrahydrofolate reductase (EC 1.5.1.20). McbR denotes a TetR-type transcriptional regulator of sulfur metabolism (Genbank accession no: AAP45010). MetQ denotes a D-methionine binding lipoprotein.

In another preferred embodiment, a methionine-producing starting organism can be derived from the wild type Coryneform bacteria and preferably from wild type C. glutamicum bacteria which contains genetic alterations in at least one of the following genes:

ask^{fbr}, hom^{fbr}, metH, metA (also referred to as metX), metY (also referred to as metZ), hst^{mutated} and metF and tkt wherein the genetic alterations lead to over expression of any of these genes in combination with genetic alterations in at least one of the following genes: mcbR and metQ wherein the genetic alterations decrease expression of any of these genes where the combination results in increased methionine production by the microorganism relative to methionine production absence of the combination. In a preferred embodiment such a methionine producing starter organism will contain genetic alterations simultaneously in ask^{fbr}, hum^{fbr}, metH, metA (also referred to as metX), metY (also referred to as metZ), hsk^{mutated} and metF and tkt wherein the genetic alterations lead to over expression of any of these genes in combination with genetic alterations in mcbR and metQ wherein the genetic alterations decrease expression of any of these genes where the combination results in increased methionine production by the microorganisms relative to methionine production absent of the combination.

In these starting organisms, the endogenous copies of ask, hum and hsk are typically replaced as described above while the endogenous copies of mcbR and metQ are typically functionally disrupted or deleted. A C. glutamicum strain which includes these genetic alterations is e.g. C. glutamicum M2543. The person skilled in the art will be aware that alternative genetic alterations to those being described below specifically for a generation of C. glutamicum M2543 can also be used to achieve over expression of ask^{fbr}, hom^{fbr}, metH, metA (also referred to as metX), metY (also referred to as metZ), hsk^{mutated}, metF and tkt and reduced expression mcbR and metQ.

For the purposes of the present invention, tkt denotes Transketolase.

As mentioned above, the present invention relates to a microorganism with increased efficiency of vitamin B12 uptake. In an aspect thereof the present invention relates to a microorganism with a deregulated vitamin B12 uptake system. Typically, such a vitamin B12 uptake system comprises nucleic acid sequences encoding at least one negative regulatory protein and/or at least one ABC-type transporter protein. In a preferred embodiment, the present invention relates to a microorganism with a deregulated vitamin B12 uptake system wherein the vitamin B12 uptake system comprises nucleic acid sequences encoding at least one negative regulatory protein and/or least one ABC-type transporter protein wherein said nucleic acid sequences encoding the at least one negative regulatory protein and at least one ABC-type transporter protein are organised as an operon such that said at least one negative regulatory protein modulates expression of said at least one ABC-type transporter protein.

An example of the invention thus relates to microorganisms which comprise a deregulated vitamin B12 uptake system which comprises an operon with nucleic acid sequences encoding at least one negative regulatory protein of SEQ ID No. 2 or functional homologous or fragments thereof and at least one ABC-type transporter protein comprising sub-units of SEQ ID Nos. 4, 6 and 8 or functional homologues or fragments thereof.

In the aforementioned microorganisms the amount and/or activity of said at least one negative regulatory protein may be at least partially reduced by genetic alteration compared to a respective starting organism not displaying said genetic alteration. Alternatively or in addition, the amount and/or activity of at least one and preferably of all subunits of the ABC-type transporter may be increased by genetic alteration compared to a respective starting organism not displaying said genetic alteration.

In a preferred embodiment, the microorganisms and methods in accordance with the invention are characterized in that additionally the amount and/or activity of one or more of the following factors functional homologues and/or functional fragments thereof is increased in comparison to a starting organism:
metA/X,
metZ/Y,
metF,
metH,
thrA,
metE,
   and/or the amount and/or activity of one or more of the following factors functional homologues and/or functional fragments thereof is decreased in comparison to a starting organism:
metK,
thrB.

Such microorganisms and methods are particularly useful for the production of methionine.

In a particularly preferred embodiment the amount and/or activity of all of the afore-mentioned factors metA/X, metZ/Y, metF, metH, thrA and metE is additionally increased and the amount and the activity of metK and thrB is additionally decreased.

MetA/X refers to a gene coding for an enzyme catalyzing the transfer of an acetyl or succinyl group from the activated acetyl-coenzyme A or the respective succinyl-coenzyme A to the OH group of homoserine to yield o-acetyl-homoserine or o-succinyl-homoserine (Genbank accession: AF052652)

MetZ/Y refers to a gene coding for an enzyme catalyzing the transfer of sulfide or methyl mercaptane to o-acetyl-homoserine or o-succinyl-homoserine, to yield homocysteine. The enzyme metZ/Y utilizes pyridoxal-phosphate as a cofactor (Genbank accession: AF220150)

MetF relates to a gene coding for an enzyme catalyzing the reduction of methylene tetrahydrofolate to methyl tetrahydrofolate utilizing NADPH or NADH as a cofactor and hydrid donor (EC 1.7.99.5, Genbank accession: AAH68531)

MetH relates to a gene coding for an enzyme catalyzing the methyl transfer from methyl tetrahydrofolate on homocysteine utilizing hydroxycobalamin as a cofactor and SAM as a second cofactor (EC 2.1.1.13, Genbank accession: Cgl1507).

ThrA (Homoserine dehydrogenase) relates to a gene coding for an enzyme catalyzing the reduction of asparto semialdehyde utilizing NADPH or NADH as a cofactor (EC 1.1.1.3, Genbank accession: Cgl1183, AAT03321, AAH68417, AEB13106). The enzyme can be used in a mutated form

ThrB (Homoserine kinase) relates to a gene coding for an enzyme catalyzing the phosphorylation of homoserine to phospho homoserine utilizing ATP as a cofactor (EC 2.7.1.39, Genbank accession: Cgl1183). The enzyme can be used in a mutated form

MetE relates to a gene coding for an enzyme catalyzing the methyl transfer from methyl tetrahydrofolate on homocysteine utilizing SAM as a cofactor (EC 2.1.1.14, Genbank accession: Cgl1139)

MetK relates to a gene coding for an enzyme catalyzing the transfer of S-adenosyl-residue on methionine utilizing ATP as a cofactor S-adenosylmethionine synthetase (EC 2.5.1.6, Genbank accession: Cgl1603)

These additional modifications can, of course, also be introduced into the above-mentioned starting organisms.

The term “decreasing the amount” of at least one protein such as a negative regulatory protein of the btu operon of C. glutamicum compared to a starting organism in the context of the present invention means that a Coryneform bacterium is genetically modified to express a lower amount of such a protein. This may for example be achieved by chromosomal deletion (see below).

The term “decreasing the activity” of at least one protein such as the negative regulatory protein of the btu operon of C. glutamicum refers to the situation that at least one mutation is introduced into the respective wall-type sequences of such a protein which leads to production of a variant thereof wherein the mutated version is expressed instead for the wild type protein. However, because of the mutation, the mutated protein loses it e.g. negative regulatory function so that even though comparable amounts in terms of protein are expressed, the activity of the respective protein is lowered.

The amount of such a protein may also be decreased by replacing the endogenous promoter with a promoter that drives expression less pronounced.

The term “increasing the amount” of at least one protein compared to a starting organism in the context of the present invention means that a Coryneform bacterium, and preferably C. glutamicum, is genetically modified to express a higher amount of at least one of the above-mentioned proteins. It is to be understood that increasing the amount of at least one protein refers to a situation where the amount of functional protein is increased. A protein in the context of the present invention is considered to be functional if it is capable of performing the same reaction as the endogenous protein. There are various options to increase the amount of a protein in Coryneform bacteria and preferably in C. glutamicum which are well known to the person skilled in the art. These options include increasing the copy number of the nucleic acid sequences which encode the above-mentioned proteins, increasing transcription and/or translation of such nucleic acid sequences. These various options will be discussed in more detail below.

The term “increasing the activity” of at least one protein refers to the situation that at least one mutation is introduced into the respective wild-type sequences of the above-mentioned protein which leads to improved import of vitamin B12 compared to a situation where the same amount of wild-type proteins is expressed.

Of course, the approaches of increasing the amount and/or activity of at least one protein ca be combined. Thus, it is for example possible to replace the endogenous copy of at least one protein in Coryneform bacteria with a mutant that encodes for a mutated version thereof. If transcription of this mutated copy is set under the control of the strong promoter, the amount and the activity of the respective protein is increased. It is understood that in this case the protein must still be capable of performing the function in which it usually participates.

Thus, one can e.g. increase the amount of the sub-units of the C. glutamicum ABC-type transporter in C. glutamicum by over expressing the respective C. glutamicum SEQ ID Nos. 4, 6 and 8 from an autonomously replicating vector or from an additionally inserted chromosomal copy (see below) or one may use the corresponding proteins from e.g. Bacillus subtilus or E. coli and over-express the proteins by e.g. use of an autonomously replicable vector.

In some circumstances, it may be preferable to use the endogenous proteins, as the endogenous coding sequence of e.g. C. glutamicum are already optimized with respect to its codon usage for expression.

In a preferred embodiment of the invention, the amount and/or activity of at least one ABC-type transporter protein of the vitamin B12 uptake system is increased in C. glutamicum.

As mentioned above, a preferred embodiment of the present invention refers to a microorganism which is genetically modified to provide increased efficiency of uptake of vitamin B12 wherein said increased efficiency is achieved by deregulated vitamin B12 uptake system with the vitamin B12 uptake system comprising nucleic acids encoding at least one negative regulatory protein and/or at least one ABC-type transporter protein which are organised as an operon such that said at least one negative regulatory protein modulates expression of said at least one ABC-type transporter protein.

A preferred embodiment relates to microorganisms selected from Coryneform bacteria with C. glutamicum being preferred.

In a particularly preferred aspect of this latter embodiment of the invention, the vitamin B12 uptake system is an operon comprising the nucleic acid sequences for a putative negative regulatory protein designated hereinafter btuR2 of SEQ ID No. 2 or for its functional homologues or fragments and preferably in C. glutamicum at least one ABC-type transporter protein being made from three subunits corresponding to SEQ ID Nos. 4, 6 and 8 or for its functional homologues or fragments. Thus, in a preferred embodiment which relates to C. glutamicum derived microorganisms, the vitamin B12 uptake system is an operon of four genes with one nucleic acid sequence encoding a negative regulatory protein such as btuR and the other three nucleic acid sequences encoding the components of an ABC-type transporter protein. The negative regulatory protein modulates expression of said ABC-type transporter proteins.

The nucleic acid sequence of C. glutamicum negative regulatory protein btuR2 is depicted in SEQ ID No. 1. The corresponding amino acid sequence is depicted in SEQ ID No. 2. The gene bank accession number (http//www.ncbi.nlm.nih.gov/) is NCgl2034 or gene ID:1020066 for the gene and NP 601315.1 for the protein.

The nucleic acid sequence for subunit A of an ABC-type transporter protein of the operon constituting a vitamin B12 uptake system in C. glutamicum depicted in SEQ ID No. 3. The gene is designated as btuF2. The corresponding amino acid sequence is depicted in SEQ ID No. 4. The gene bank accession number is accession NC_{—}006958.1 or geneID:3345625 for the gene and NP 601313.1 for the protein.

The nucleic acid sequence for subunit B of the ABC-type transporter protein of the operon constituting a vitamin B12 uptake system in C. glutamicum is depicted in SEQ ID No. 5. The gene designation is btuC2. The amino acid sequence is depicted in SEQ ID No. 6. The gene bank accession number is NCgl2032 or gene ID:1020064 for the gene and NP 601312.1 for the protein.

The nucleic acid sequence for subunit C of the ABC-type transporter protein of the operon constituting a vitamin B12 uptake system in C. glutamicum is depicted in SEQ ID No. 7. The gene designation is btuD2. The amino acid sequence is depicted in SEQ ID No. 8. The gene bank accession number is NCgl2031 or gene ID:1020063 for the gene and NP 601311.1 for the protein

The corresponding functional homologues of the above mentioned C. glutamicum sequences constituting a vitamin B12 uptake system in C. glutamicum can easily be identified by the skilled person for other organisms by homology analysis. This can be done be determining percent identity between amino acid or nucleic acid sequences for putative homologues in the sequences for the genes or proteins encoded by e.g. nucleic acid sequences for btuR2, butF2 and any of the genes and proteins encoded thereby mentioned hereinafter.

Percent identity may be determined, for example, by visual inspection or by using algorithm-based homology.

For example, in order to determine percent identity of two amino acid sequences, the algorithm will align the sequences for optimal comparison purposes (e.g., gaps can be introduced in the amino acid sequence of one protein for optimal alignment with the amino acid sequence of another protein). The amino acid residues at corresponding amino acid positions are then compared. When a position in one sequence is occupied by the same amino acid residue as the corresponding position in the other, then the molecules are identical at that position. The percent identity between the two sequences is a function of the number of identical positions shared by the sequences (i.e., % identity=# of identical positions/total # of positions multiplied by 100).

Various computer programs are known in the art for these purposes. For example, percent identity of two nucleic acid or amino acid sequences can be determined by comparing sequence information using the GAP computer program described by Devereux et al. (1984) Nucl. Acids. Res., 12:387 and available from the University of Wisconsin Genetics Computer Group (UWGCG). Percent identity can also be determined by aligning two nucleic acid or amino acid sequences using the Basic Local Alignment Search Tool (BLAST™) program (as described by Tatusova et al. (1999) FEMS Microbiol. Lett., 174:247.

At the filing date of this patent application, a standard software package providing the BLAST programme can be found on the BLAST website of the NCBI (http://www.ncbi.nlm.nih.gov/BLAST/). For example, if one uses any of the aforementioned SEQ IDs, one can either perform a nucleic acid sequence- or amino sequence-based BLAST search and identify closely related homologs of the respective enzymes in e.g. E. coli, S. cervisiae, Bacillus subtilis, etc. For example, for nucleic acid sequence alignments using the BLAST™ program, the default settings are as follows: reward for match is 2, penalty for mismatch is −2, open gap and extension gap penalties are 5 and 2 respectively, gap.times.dropoff is 50, expect is 10, word size is 11, and filter is OFF. The latter algorithm is preferred.

Comparable sequence searches and analysis can be performed at the EMBL database (http://www.embl.org) or the Expasy homepage (http://www.expasy.org/). All of the above sequences searches are typically performed with the default parameters as they are pre-installed by the database providers at the filing date of the present application. Homology searches may also routinely be performed using software programmes such as the laser gene software of DNA Star, Inc., Madison, Wis., USA, which uses the CLUSTAL method (Higgins et al. (1989), Comput. Appl. Biosci., 5(2) 151).

The skilled person understands that two proteins will likely perform the same function (e.g. provide the same enzymatic activity) if they share a certain degree of identity as described above. A typical lower limit on the amino acid level is typically at least about 50% identity. On the nucleic acid level, the lower limit is typically at least 40%.

Preferred identity grades for both type of sequences are at least about 55%, at least about 60% or least about 70%. More preferred identity levels are at least about 80%, at least about 90% or at least about 95%. These identity levels are considered to be significant.

As used herein, the terms “homology” and “homologous” are not limited to designate proteins having a theoretical common genetic ancestor, but includes proteins which may be genetically unrelated that have, none the less, evolved to perform similar functions and/or have similar structures. The requirement that the homologues should be functional means that the homologues herein described encompasse proteins that have substantially the same function as the reference protein. For proteins to have functional homology, it is not necessarily required that they have significant identity in their amino acid sequences, but, rather, proteins having functional homology are so defined by having similar or identical activities, e.g., enzymatic activities.

Preferably, a protein from another organism than e.g. the host Coryneform bacteria will be considered to be a functional homolog if it shows at least significant similarity, i.e. about 50% sequence identity on the amino acid level, and performs the same function as its counterpart in the Coryneform bacterium. Functional homologues which provide the same enzymatic activity and share a higher degree of identity such as at least about 60%, at least about 70%, at least about 80% or at least about 90% sequence identity on the amino acid level are further preferred functional homologues.

The person skilled in the art knows that one can also use fragments or mutated versions of the aforementioned proteins from Coryneform bacteria and of their functional homologues in other organisms as long as these fragments and mutated versions display the same type of functional activity. Typical functionally active fragments will display N-terminal and/or C-terminal deletions while mutated versions typically comprise deletions, insertions or point mutations.

By way of example, a sequence of Bacillus subtilus will be considered to encode for a functional homologue of C. glutamicum btuR2 if it displays the above mentioned identity levels on the amino acid level to SEQ ID No. 2 and displays comparable inhibitory activity on the expression of the ABC-type transporter protein as e.g. encoded by SEQ ID Nos. 2, 4 and 6. One can also use fragments or e.g. point mutations of these sequences as long as the resulting proteins still mediate the same type of activity as the full-length proteins.

According to the present invention, deregulating a vitamin B12 uptake system and particularly those as described for C. glutamicum in the form of the btu2 operon allows the growth of microorganisms and/or improved methionine production in media that contain less vitamin B12 compared to microorganisms which do not comprise such a deregulated vitamin B12 uptake system.

Thus, microorganisms which have been genetically modified according to the invention may be grown in media to the same efficiency as microorganisms not displaying said genetic alterations with the media comprising about less than 10%, about less than 20%, about less than 30%, about less than 40%, about less than 50%, about less than 60%, about less than 70%, about less than 80%, about less than 90% or about less than 95% of vitamin B12 compared to the situation where the organism does not comprise genetic alterations to improve vitamin B12 uptake efficiency.

In a preferred embodiment, organisms genetically modified in accordance with the invention can be grown to a comparable and preferably the same efficiency as microorganisms without the genetic alteration if the media comprise less than 50%, preferably less than about 60%, more preferably less than about 70%, even more preferably even less than about 80% and even more preferably less than about 90% vitamin B12 compared to the media that are commonly used microorganisms that do not comprise genetic alterations to improve vitamin B12 uptake as described in the present invention.

The microorganisms in accordance with the invention as a consequence of their improved vitamin B12 uptake system can thus be used at lower costs to provide fine chemicals the biosynthesis of which require vitamin B12. Such fine chemicals include methionine, S-adenosyl methionine, and methionine sulfoxide.

In some embodiments, microorganisms with an improved vitamin B12 uptake efficiency as a consequence of genetic alterations as described herein may even improve production of methionine and other fine chemicals which require vitamin B12 for their biosynthesis in microorganisms such as Coryneform bacteria and preferably in C. glutamicum.

Improving production of methionine in Coryneform bacteria means inter alia increasing the production rate, the final titer, or the efficiency of methionine synthesis as well as increasing the amount of methionine produced.

The term “efficiency of methionine synthesis” describes the carbon yield of methionine. This efficiency is calculated as a percentage of the energy input which entered the system in the form of a carbon substrate. Throughout the invention this value is given in percent values ((mol methionine) (mol carbon substrate (⁻¹×100). The term “increased efficiency of methionine synthesis” thus relates to a comparison between the starting organism and the actual Coryneform bacterium in which the amount and/or activity of at least one of the enzymes of the pentose phosphate pathway has been increased.

Preferred carbon sources according to the present invention are sugars such as mono-, di- or polysaccharides. For example, sugars selected from the group comprising glucose, fructose, hanose, galactose, ribose, sorbose, lactose, maltose, sucrose, raffinose, starch or cellulose may serve as particularly preferred carbon sources.

The methods and Coryneform bacteria in accordance with the invention may also be used to produce more methionine compared to the starting organism.

The methods and Coryneform bacteria in accordance with the invention may also be used to produce methionine at a faster rate compared to the starting organism. If, for example, a typical production period is considered, the methods and Coryneform bacteria will allow to produce methionine at a faster rate, i.e. the same amount methionine will be produced at an earlier point in time compared to the starting organism. This particularly applies for the logarithmic growth phase.

Methods and Coryneform bacteria in accordance with the invention allow to produce at least about 3 g methionine/l culture volume if the strain is incubated in shake flask incubations. A titer of at least about 4 g methionine/l culture volume, at least about 5 g methionine/l culture volume or at least about 7 g methionine/l culture volume can be preferred if the strain is incubated in shake flask incubations. A more preferred value amounts to at least about 10 g methionine/l culture volume and even more preferably to at least about 20 g methionine/l cell mass if the strain is incubated in shake flask incubations.

Methods and Coryneform bacteria in accordance with the invention allow to produce at least about 25 g methionine/l culture volume if the strain is incubated in fermentation experiments using a stirred and carbon source fed fermentor. A titer of at least about 30 g methionine/l culture volume, at least about 35 g methionine/l culture volume or at least about 40 g methionine/l culture volume can be preferred if the strain is incubated in fermentation experiments using a stirred and carbon source fed fermentor. A more preferred value amounts to at least about 50 g methionine/l culture volume and even more preferably to at least about 60 g methionine/l cell mass if the strain is incubated in fermentation experiments using a stirred and carbon source fed fermentor.

In a preferred embodiment, the methods and microorganisms of the invention allow to increase the efficiency of methionine synthesis and/or the amount of methionine and/or the titer and/or the rate of methionine synthesis in comparison to the starting organism by at least about 2%, at least about 5%, at least about 10% or at least about 20%. In preferred embodiments the efficiency of methionine synthesis and/or the amount of methionine and/or the titer and/or the rate is increased compared to the starting organism by at least about 30%, at least about 40%, or at least about 50%. Even more preferred is an increase of at least about factor 2, at least about factor 3, at least about factor 5 and at least about factor 10. However, an increase of about 5% may already be considered to be a significant improvement.

The amount and/or activity of the above mentioned sequences as well as their functional homologues and fragments may be increased and/or decreased preferably in C. glutamicum. To this end one can either use a wild type strain such as ATCC13032 or a strain carrying further genetic modifications to increase and improve methionine biosynthesis.

Such a strain can, for example, express a feedback-resistant homoserine dehydrogenase (hom^{fbr}). Such a strain can further express a feedback-resistant aspartate kinase (ask^{fbr}) Such a strain may additionally display increased expression of methionine synthase (metH). A strain which is suitable for production of methionine and which overexpresses a feedback-resistant homoserine dehydrogenase, a feedback-resistant aspartate kinase and methionine synthase is e.g. the aforementioned DSM17322 of Example

Other C. glutamicum starting strains which can be preferably used for the purposes of the present invention carry the aforementioned modifications of DSM17322 and are further optimized with respect to methionine synthesis. Such strains may for example express increased levels of a mutated homoserine kinase (hsk^{mutatedr}), a homoserine succinyltransferase (metA), and a O-Acetylhomoserine sulfhydrylase (metY) A strain which carries all these genetic alterations is e.g. M2014 of Example 1. A particularly promising starting organism in C. glutamicum for the purposes of the present invention will therefore display increased levels of metH, metY and metA, hom^{fbr}, ask^{fbr} and hsk^{mutated}.

An example of a feedback-resistant homoserine dehydrogenase carries a S393F mutation at position 393 of SEQ ID NO. 17. This hom^{fbr} shows reduced feedback inhibition by threonine and or methionine. An example of a feedback-resistant aspartate kinase carries a T311I mutation at position 311 of SEQ ID NO. 18. This ask^{fbr} shows reduced feedback inhibition by lysine and or threonine. A homoserine kinase carrying the aforementioned functional mutation carries a T190A mutation at position 190 of SEQ ID NO. 19 or a T190S mutation at position 190 or a TTG start codon.

The C. glutamicum starting organism which may carry the aforementioned genetic alterations such as M2014 can be further improved by deleting the nucleic acid sequences for the negative regulator (mcbR) (Rey, D. et al. (2005) Mol. Microbiol., 56. 871-887, Rey, D. et al. (2003) J. Biotechnol., 103, 51-65, US2005074802) and the D-methionine binding lipoprotein (metQ) as well as by increasing expression of N5,10-methylene-tetrahydrofolate reductase (metF). A corresponding strain is described in Example 5 as OM469. Strains displaying genetic alterations that are identical to or comparable with those DSM17322, M2014 or OM469 can be preferred as C. glutamicum starting organisms.

Other strains that may be preferable starting organisms comprise in addition to the aforementioned genetic alterations of the starting strains DSM17322, M2014 or OM469 in increased activity and/or amount of transketolase (tkt). In a particularly preferred embodiment, such a starting organism may relate to a C. glutamicum organism in which the endogenous promoter preceding tkt in C. glutamicum is replaced by a strong promoter such an approach is preferably for the reason that the genes for transketolase, 6-phospho-glucono-lactonse, glucose-6-phosphate-dehydrogenase and the gene called OPCA are organised in a single operon. Thus, a strong promoter instead of the endogenous promoter in front of tkt, such as the e.g. λ_{PR} promoter allows to concomitantly increase the amount and/or activity of enzymes that are involved in the pentose phosphate pathway in addition to transketolase and which may be beneficial to methionine surfaces. A starting organism that reflects corresponding genetic alterations is M2543 as described hereinafter.

One can increase the amount of an enzyme of the pentose phosphate pathway in a Coryneform bacterium by e.g. increasing the gene copy number, i.e. the copy number of the nucleic acid sequence encoding said enzyme, by increasing transcription, by increasing translation, and/or a combination thereof.

The person skilled in the art is familiar with the type of genetic alterations that are necessary in order to increase the gene copy number of nucleic acid sequences, to increase transcription and/or to increase translation.

In general, one can increase the copy number of a nucleic acid sequence encoding a polypeptide by expressing a vector in the Coryneform bacterium which comprises the nucleic sequence encoding said polypeptide. Such vectors can be autonomously replicable so that they can be stably kept within the Coryneform bacterium. Typical vectors for expressing polypeptides and enzymes of the pentose phosphate pathway in C. glutamicum include pCliK pB and pEKO as described in Bott, M. and Eggeling, L., eds. Handbook of Corynebacterium glutamicum. CRC Press LLC, Boca Raton, Fla.; Deb, J. K. et al. (FEMS Microbiol. Lett. (1999), 175(1), 11-20), Kirchner O. et al. (J. Biotechnol. (2003), 104 (1-3), 287-299), WO2006069711 and in WO2007012078.

In another approach for increasing the copy number of nucleic acid sequences encoding a polypeptide in a Coryneform bacterium, one can integrate additional copies of nucleic acid sequences encoding such polypeptides into the chromosome of C. glutamicum. Chromosomal integration can e.g. take place at the locus where the endogenous copy of the respective polypeptide is localized. Additionally and/or alternatively, chromosomal multiplication of polypeptide encoding nucleic acid sequences can take place at other loci in the genome of a Coryneform bacterium. In case of C. glutamicum, there are various methods known to the person skilled in the art for increasing the gene copy number by chromosomal integration. One such method makes e.g. use of the vector pK19 sacB and has been described in detail in the publication of Schafer A, et al. J. Bacteriol. 1994 176(23): 7309-7319. Other vectors for chromosomal integration of polypeptide-encoding nucleic acid sequences include or pCLIK int sacB as described in WO2005059093 or WO2007011845.

Increasing the amount of at least one enzyme of the pentose phosphate pathway can also be achieved by increasing transcription of the nucleic acid sequences encoding the respective enzymes. Increased transcription will lead to more mRNA and ultimately to a higher amount of translated protein.

The person skilled in the art is aware that one can increase transcription of a coding sequence in Coryneform bacteria by numerous approaches. Thus, one can increase transcription by using strong promoters and/or strong enhancer elements. One may also use transcriptional activators such as e.g. aptamers or overexpress transcription factors. The use of strong promoters can be preferred in the context of the present invention.

A promoter is considered to be a “strong promoter” in the context of the present invention if it provides a higher degree of transcription for a nucleic acid sequence encoding a respective polypeptide than the endogenous promoter that precedes the respective nucleic acid sequence in the wild-type situation.

For the purposes of the present invention, the use of the following promoter can be considered: P_{SOD} (SEQ ID NO. 9), P_{groES} (SEQ ID NO. 10), P_{EFTu} (SEQ ID NO. 11) and λP_{L} (SEQ ID NO. 12). These promoters are commonly used in C. glutamicum to over-express polypeptides and the strength of the promoters is considered in some contexts, but not necessarily all contexts, to have the following order:

### λP_{L}>P_{EFTu}>P_{SOD}>P_{GRoES}.

Improvement of translation can be achieved e.g. by optimising the codon usage of the nucleic acid sequences encoding for the respective enzymes. If one uses the nucleic acid sequences of the host enzymes, adaptation of the codon usage is typically not necessary but can be also applied. If however, the amount of e.g. ABC-type transporter protein is to be increased by over-expression of the respective sequences of E. coli in C. glutamicum, it may be worth considering adapting the coding sequence of the E. coli proteins to the codon usage of C. glutamicum.

In some embodiments of the invention, it can be preferred to increase the copy numbers of the nucleic acid sequences encoding subunits of an ABC-type transporter protein as observed e.g. in the btu operon of C. glutamicum by integrating the respective nucleic acid sequences in multiple copies at the position of the endogenous gene in the chromosome of the respective Coryneform bacteria and preferably in C. glutamicum. This approach usually preserves the genomic integrity of the genome as much as possible.

The person skilled in the art is also familiar with various methods for down-regulating the amount and/or activity of proteins being encoded by nucleic acid sequences such as e.g. the negative regulatory protein btuR2 of the btu2 operon in C. glutamicum. A typical approach for down regulating the amount and/or activity of e.g. btuR2 would be to genetically disrupt the endogenous gene encoding this vector so that no functional produced is produced any longer. In an alternative embodiment, one may introduce mutations in the endogenous sequence of e.g. SEQ ID No. 1 such that the resulting protein does not excerpt its negative inhibitory effect on the expression of the at least one ABC-type transporter protein or at least to a lesser degree. One may also use an approach where a promoter that is weaker than the endogenous promoter is placed in front of the nucleic acid sequence encoding the negative regulatory protein such as btuR2 in the btu operon in C. glutamicum.

A promoter can be made weaker by changing one base at a time to make the promoter less similar to the consensus promoter at the “−35” or “−10” region. A consensus “−35” region may have the sequence TTGACA, and a consensus “−10” region has the sequence TATAAT.

Of course, the person skilled in the art is also aware that one may combine the above mentioned approaches. Thus, one can envisage a microorganism preferably being selected from Corynebacterium and even more preferably from C. glutamicum wherein the nucleic acid sequences encoding btuR2 or its counterparts are disrupted and where expression of the ABC-type transporter protein being encoded by e.g. SEQ ID Nos. 2, 6 and 8 or its counterparts is over expressed by e.g. use of a strong promoter.

Table 1 below gives an overview on gene bank accession numbers of sequences of enzymes or proteins and the genes that encode them, to be discussed hereinafter in more detail. The gene bank accession numbers recited refer to the GenBank or other public databases which can be found or accessed at the website http://www.ncbi.nlm.nih.gov/. Many homologs of any of the genes or proteins listed in the below table can be found by using the “BLAST” programs found at the same website using a sequence from the table below as the “query”, as is well known in the art.

TABLE 1
Proteins specifically mentioned herein and homologues thereof

| Proteins | Gene bank accession number | Organism |
|---|---|---|
| btuR2 | NCgl2034, geneI: 1020066, NP 601315.1 | Corynebacterium |
| | | glutamicum and others |
| btuF2 | NCgl2033, geneID: 1020065, NP 601313.1 | Corynebacterium |
| | | glutamicum and others |
| btuC2 | NCgl2032, geneID: 1020064, NP 6013152.2 | Corynebacterium |
| | | glutamicum and others |
| btuD2 | NCgl2031, geneID: 1020063, NP 601311.1 | Corynebacterium |
| | | glutamicum and others |
| Glucose-6-phosphate- | Cgl1576, BAB98969, NCgl1514, NCgl1514, cg1778, CE1696, | Corynebacterium |
| dehydrogenase | DIP1304, jk0994, RHA1_ro07184, nfa35750, MSMEG_3101, | glutamicum and others |
| | Mmcs_2412, MAP1176c, Mb1482c, MT1494, Rv1447c, | |
| | SAV6313, Acel_1124, SCO1937, MAV_3329, Lxx11590, | |
| | BL0440, Arth_2094, Tfu_2005, itte weitere angeben | |
| OPCA protein | Cgl1577, NP_738307.1, NP_939658.1, YP_250777.1, YP_707105.1, | Corynebacterium |
| | YP_119788.1, ZP_01192082.1, NP_335942.1, ZP_01276169.1, | glutamicum and others |
| | NP_215962.1, ZP_01684361.1, YP_887415.1, | |
| | ZP_01130849.1, YP_062111.1, ZP_00615668.1, YP_953530.1, | |
| | ZP_00995403.1, YP_882512.1, NP_960109.1, YP_290062.1, | |
| | YP_831573.1, NP_827488.1, YP_947837.1, NP_822945.1, | |
| | NP_626203.1, NP_630735.1, CAH10103.1, ZP_00120910.2, | |
| | NP_695642.1, YP_909493.1, YP_872881.1, YP_923728.1, | |
| | YP_056265.1, ZP_01648612.1, ZP_01430762.1, ZP_00569428.1, | |
| | YP_714762.1, YP_480751.1, NP_301492.1, | |
| | YP_642845.1, ZP_00767699.1 | |
| 6- | Cgl1578, NCgl1516, NCgl1516, cg1780, CE1698, DIP1306, | Corynebacterium |
| phosphogluconolactonase | Mmcs_2410, MSMEG_3099, Mb1480c, MT1492, Rv1445c, | glutamicum and others |
| | MAV_3331, RHA1_ro07182, nfa35770, MAP1174c, ML0579, | |
| | jk0996, Tfu_2007, FRAAL4578, SAV6311, SCO1939, SCC22.21, | |
| | TW464 | |
| 6-phospho-gluconate- | Cgl1452, BAB98845, NCgl1396, cgl1452, NCgl1396, cg1643, | Corynebacterium |
| dehydrogenase | DIP1213, CE1588, jk0912, RHA1_ro07246, nfa11750, Mmcs_2812, | glutamicum and others |
| | MSMEG_3632, MT1892, Rv1844c, MAV_2871, | |
| | MAP1557c, ML2065, SAV724, SCO0975, SCBAC19F3.02, | |
| | BL0444, Lxx17380, Arth_2449, Mb1875c, OB0185 | |
| | Bitte weitere angeben | |
| Ribulose-5-P-epimerase | Cgl1598, cg1801, CE1717, DIP1320, MSMEG_3066, Mb1443, | Corynebacterium |
| | MT1452, Rv1408, MAV_3370, ML0554, jk1011, MAP1135, | glutamicum and others |
| | RHA1_ro07167, Mmcs_2385, nfa36030, SCO1464, | |
| | SAV6880, FRAAL5223, Acel_1276, BL0753 | |
| Ribose-5-P-isomerase | Cgl2423, cg2658, CE2318, DIP1796, nfa13270, jk0541, RHA1_ro01378, | Corynebacterium |
| | MSMEG_4684, Mmcs_3599, Mb2492c, Rv2465c, | glutamicum and others |
| | MT2540, ML1484, MAV_1707, MAP2285c, SCO2627, | |
| | SAV5426, Tfu_2202, Arth_2408, PPA1624, Francci3_1162 | |
| Transketolase | Cgl1574, YP_225858, cg1774, CE1694, DIP1302, jk0992, nfa35730, | Corynebacterium |
| | RHA1_ro07186, MSMEG_3103, MAP1178c, ML0583, | glutamicum and others |
| | MAV_3327, Mb1484c, MT1496, Rv1449c, Mmcs_2414, | |
| | Tfu_2002, Arth_2097, Lxx11620, SAV1766, SCO1935, Acel_1127 | |
| Transaldolase | Cgl1575, cg1776, CE1695, DIP1303, jk0993, Mmcs_2413, | Corynebacterium |
| | MSMEG_3102, MAP1177c, RHA1_ro07185, MAV_3328, | glutamicum and others |
| | Mb1483c, Rv1448c, MT1495, nfa35740, ML0582, Arth_2096, | |
| | Lxx11610, SAV1767, Tfu_2003, SCO1936, Francci3_1648 | |
| Methylene | Cgl2171, CE2066, cg2383, DIP1611, jk0737, RHA1_ro01105, | C. glutamicum and |
| tetrahydrofolate | nfa17400, Tfu_1050, Acel_0991, SAV6100, SCO2103, FRAAL2163, | others |
| reductase (metF) | Francci3_1389, aq_1429, TTC1656, TTHA0327, | |
| | ELI_10095, CT1368, Sala_0035, DP1612, Pcar_1732 | |
| cob(I)alamin dependent | Cgl1507, CE1637, cg1701, DIP1259, RHA1_ro00859, nfa31930, | C. glutamicum and |
| methionine synthase | Rv2124c, Mb2148c, ML1307, SCO1657, Tfu_1825, SAV6667, | others |
| (metH) | Arth_3627, Acel_1174, MT2183, GOX2074, tll1027, | |
| | GbCGDNIH1_0151, Rru_A1531, alr0308, slr0212 | |
| O-acetylhomoserine | Cgl0653, NCgl0625, cg0755, CE0679, DIP0630, | C. glutamicum and |
| sulfhydrolase (metY) | jk1694, MAP3457, Mb3372, MT3443, Rv3340, | others |
| | nfa35960, Lxx18930, Tfu_2823, CAC2783, GK0284, | |
| | BH2603, lmo0595, lin0604, LMOf2365_0624, ABC0432, | |
| | TTE2151, BT2387, STH2782, str0987, stu0987, BF1406, | |
| | SH0593, BF1342, lp_2536, L75975, OB3048, BL0933, | |
| | LIC11852, LA2062, BMAA1890, BPSS0190, | |
| | SMU.1173, BB1055, PP2528, PA5025, PBPRB1415, | |
| | GSU1183, RPA2763, WS1015, TM0882, VP0629, | |
| | BruAb1_0807, BMEI1166, BR0793, CPS_2546, | |
| | XC_1090, XCC3068, plu3517, PMT0875, SYNW0851, | |
| | Pro0800, CT0604, NE1697, RB8221, bll1235, | |
| | syc1143_c, ACIAD3382, ebA6307, RSc1562, | |
| | Daro_2851, DP2506, DR0873, MA2715, PMM0642, | |
| | PMN2A_0083, IL2014, SPO1431, ECA0820, | |
| | AGR_C_2311, Atu1251, mlr8465, SMc01809, CV1934, | |
| | SPBC428.11, PM0738, SO1095, SAR11_1030, | |
| | PFL_0498, CTC01153, BA_0514, BCE5535, BAS5258, | |
| | GBAA5656, BA5656, BCZK5104, TTHA0760, | |
| | TTC0408, BC5406, BT9727_5087, HH0636, YLR303W, | |
| | ADL031W, CJE1895, spr1095, rrnAC2716, orf19.5645, | |
| | Cj1727c, VNG2421G, PSPPH_1663, XOO1390, | |
| | Psyr_1669, PSPTO3810, MCA2488, TDE2200, FN1419, | |
| | PG0343, Psyc_0792, MS1347, CC3168, Bd3795, | |
| | MM3085, 389.t00003, NMB1609, SAV3305, NMA1808, | |
| | GOX1671, APE1226, XAC3602, NGO1149, ZMO0676, | |
| | SCO4958, lpl0921, lpg0890, lpp0951, EF0290, BPP2532, | |
| | CBU2025, BP3528, BLi02853, BL02018, BG12291, | |
| | CG5345-PA, HP0106, ML0275, jhp0098, At3g57050, | |
| | 107869, HI0086, NTHI0100, SpyM3_0133, SPs0136, | |
| | spyM18_0170, M6_Spy0192, SE2323, SERP0095, | |
| | SPy0172, PAB0605, DDB0191318, ST0506, F22B8.6, | |
| | PTO1102, CPE0176, PD1812, XF0864, SAR0460, | |
| | SACOL0503, SA0419, Ta0080, PF1266, MW0415, | |
| | SAS0418, SSO2368, PAE2420, TK1449, 1491, | |
| | TVN0174, PH1093, VF2267, Saci_0971, VV11364, | |
| | CMT389C, VV3008 | |
| Aspartate kinase (ask) | Cgl0251, NCgl0247, CE0220, DIP0277, jk1998, nfa3180, | C. glutamicum and |
| | Mb3736c, MT3812, Rv3709c, ML2323, MAP0311c, | others |
| | Tfu_0043, Francci3_0262, SCO3615, SAV4559, | |
| | Lxx03450, PPA2148, CHY_1909, MCA0390, | |
| | cbdb_A1731, TWT708, TW725, Gmet_1880, DET1633, | |
| | GSU1799, Moth_1304, Tcr_1589, Mfla_0567, | |
| | HCH_05208, PSPPH_3511, Psyr_3555, PSPTO1843, | |
| | CV1018, STH1686, NMA1701, Tbd_0969, NMB1498, | |
| | Pcar_1006, Daro_2515, Csal_0626, Tmden_1650, | |
| | PA0904, PP4473, Sde_1300, HH0618, NGO0956, | |
| | ACIAD1252, PFL_4505, ebA637, Noc_0927, WS1729, | |
| | Pcryo_1639, Psyc_1461, Pfl_4274, LIC12909, LA0693, | |
| | Rru_A0743, NE2132, RB8926, Cj0582, Nmul_A1941, | |
| | SYN_02781, TTHA0534, CJE0685, BURPS1710b_2677, | |
| | BPSL2239, BMA1652, RSc1171, TTC0166, RPA0604, | |
| | BTH_I1945, Bpro_2860, Rmet_1089, Reut_A1126, | |
| | RPD_0099, Bxe_A1630, Bcep18194_A5380, aq_1152, | |
| | RPB_0077, Rfer_1353, RPC_0514, BH3096, BLi02996, | |
| | BL00324, amb1612, tlr1833, jhp1150, blr0216, | |
| | Dde_2048, BB1739, BPP2287, BP1913, DVU1913, | |
| | Nwi_0379, ZMO1653, Jann_3191, HP1229, Saro_3304, | |
| | Nham_0472, CBU_1051, slr0657, SPO3035, | |
| | Synpcc7942_1001, BG10350, BruAb1_1850, | |
| | BAB1_1874, BMEI0189, BT9727_1658, syc0544_d, | |
| | BR1871, gll1774, BC1748, mll3437, BCE1883, | |
| | ELI_14545, RSP_1849, BCZK1623, BAS1676, | |
| | BA_2315, GBAA1811, BA1811, Ava_3642, alr3644, | |
| | PSHAa0533, AGR_L_1357, Atu4172, lin1198, | |
| | BH04030, PMT9312_1740, SMc02438, CYA_1747, | |
| | RHE_CH03758, lmo1235, LMOf2365_1244, | |
| | PMN2A_1246, CC0843, Pro1808, BQ03060, PMT0073, | |
| | Syncc9902_0068, GOX0037, CYB_0217 | |
| Homoserine | Cgl0652, CE0678, CE0678, cg0754, DIP0623, jk1695, nfa9220, | C. glutamicum and |
| Succinyltransferase | RHA1_ro06236, MAP3458, MAV_4316, MSMEG_1651, | others |
| (metA) | Mmcs_1207, ML0682, Mb3373, Rv3341, MT3444, Tfu_2822, | |
| | Arth_1318, Francci3_2831, Lxx18950, FRAAL4363, Cag_1206, | |
| | Adeh_1400, Plut_0593, CT0605, CHY_1903, Moth_1308, | |
| | Ava_4076, STH1685, SRU_0480, Mbur_0798, Mhun_2201, | |
| | RPC_4281 Msp_0676 | |
| homoserine | Cgl1183, CE1289, cg1337, DIP1036, jk1352, nfa10490, RHA1_ro01488, | C. glutamicum and |
| dehydrogenase (hom) | MSMEG_4957, Mmcs_3896, MAV_1509, Mb1326, | others |
| | Rv1294, MT1333, MAP2468c, ML1129, SAV2918, SCO5354, | |
| | FRAAL5951, Francci3_3725, Tfu_2424, Acel_0630 | |
| Homoserine kinase (hsk) | Cgl1184, cg0307, CE0221, DIP0279, jk1997, RHA1_ro04292, | C. glutamicum and |
| | nfa3190, Mmcs_4888, MSMEG_6256, MAP0310c, MAV_0394, | others |
| | Mb3735c, MT3811, Rv3708c, Acel_2011, ML2322, PPA0318, | |
| | Lxx03460, SCO2640, SAV5397, CC3485 | |
| D-methionine binding | YP_224930, NP_599871, NP_737241, NP_938985, NP_938984, | C. glutamicum and |
| lipoprotein (metQ) | YP_701727, YP_251505, YP_120623, YP_062481, YP_056445, | others |
| | ZP_00121548, NP_696133, YP_034633, YP_034633, | |
| | YP_081895, ZP_00390696, YP_016928, YP_026579, | |
| | NP_842863, YP_081895, ZP_00240243, NP_976671 | |
| mcbR | cg3253, CE2788, DIP2274, jk0101, nfa21280, MSMEG_4517Lxx16190, | C. glutamicum and |
| | SCO4454, Bcep18194_A3587, Bamb_0404, Bcen2424_0499, | others |
| | Bcen_2606, Ava_4037, BTH_I2940, RHA1_ro02712, | |
| | BMA10299_A1735, BMASAVP1_A0031, BMA2807, | |
| | BURPS1710b_3614 | |

The above accession numbers are the official accession numbers of Genbank or are synonyms for accession numbers which have cross-references at Genbank. These numbers can be searched and found at http://www.ncbi.nlm.nih.gov/.

A general overview is given below on how to increase and decrease the amount and/or activity of polypeptides and genes in C. glutamicum. The skilled person can rely on this information when putting embodiments besides those disclosed in the examples below into practice.

Increasing or Introducing the Amount and/or Activity

With respect to increasing the amount, two basic scenarios can be differentiated. In the first scenario, the amount of the enzyme is increased by expression of an exogenous version of the respective protein. In the other scenario, expression of the endogenous protein is increased by influencing the activity of e.g. the promoter and/or enhancers ribosomal binding sites element and/or other regulatory activities that regulate the activities of the respective proteins either on a transcriptional, translational or post-translational level.

Thus, the increase of the activity and the amount of a protein may be achieved via different routes, e.g. by switching off inhibitory regulatory mechanisms at the transcriptional, translational, and protein level or by increase of gene expression of a nucleic acid coding for these proteins in comparison with the starting organism, e.g. by inducing endogenous transketolase by a strong promoter and/or by introducing nucleic acids encoding for transketolase.

In one embodiment, the increase of the amount and/or activity of the enzymes of Table 1 is achieved by introducing nucleic acids encoding the enzymes of Table 1 or into the Coryneform bacteria, preferably C. glutamicum.

In principle, every protein of different organisms with an enzymatic activity of the proteins listed in Table 1 or 2, can be used. With genomic nucleic acid sequences of such enzymes from eukaryotic sources containing introns, already processed nucleic acid sequences like the corresponding cDNAs are to be used in the case as the host organism is not capable or cannot be made capable of splicing the corresponding mRNAs. All nucleic acids mentioned in the description can be, e.g., an RNA, DNA or cDNA sequence.

According to the present invention, increasing or introducing the amount of a protein typically comprises the following steps:
a) production of a vector comprising the following nucleic acid sequences, preferably DNA sequences, in 5′-3′-orientation:
   a promoter sequence functional in the organisms of the invention,
   operatively linked thereto a DNA sequence coding for a protein of e.g. Table 1, functional homologues, functional fragments or functional mutated versions thereof
   a termination sequence functional in the organisms of the invention
b) transfer of the vector from step a) to the organisms of the invention such as C. glutamicum and, optionally, integration into the respective genomes.

As set out above, functional fragments relate to fragments of nucleic acid sequences coding for enzymes of e.g. Table 1 or 2, the expression of which still leads to proteins having the enzymatic activity of the respective full length protein.

The above-mentioned method can be used for increasing the expression of DNA sequences coding for enzymes of e.g. Table 1 or functional fragments thereof. The use of such vectors comprising regulatory sequences, like promoter and termination sequences are, is known to the person skilled in the art. Furthermore, the person skilled in the art knows how a vector from step a) can be transferred to organisms such as C. glutamicum and which properties a vector must have to be able to be integrated into their genomes.

According to the present invention, an increase of the gene expression of a nucleic acid encoding an enzyme of Table 1 is also understood to be the manipulation of the expression of the endogenous respective endogenous enzymes of an organism, in particular of C. glutamicum. This can be achieved, e.g., by altering the promoter DNA sequence for genes encoding these enzymes. Such an alteration, which causes an altered, preferably increased, expression rate of these enzymes can be achieved by replacement with strong promoters and by deletion and/or insertion of DNA sequences.

An alteration of the promoter sequence of endogenous genes usually causes an alteration of the expressed amount of the gene and therefore also an alteration of the activity detectable in the cell or in the organism.

Furthermore, an altered and increased expression, respectively, of an endogenous gene can be achieved by a regulatory protein, which does not occur in the transformed organism, and which interacts with the promoter of these genes. Such a regulator can be a chimeric protein consisting of a DNA binding domain and a transcription activator domain, as e.g. described in WO 96/06166.

A further possibility for increasing the activity and the content of endogenous genes is to up-regulate transcription factors involved in the transcription of the endogenous genes, e.g. by means of overexpression. The measures for overexpression of transcription factors are known to the person skilled in the art.

The expression of endogenous enzymes such as those of Table 1 can e.g. be regulated via the expression of aptamers specifically binding to the promoter sequences of the genes. Depending on the aptamer binding to stimulating or repressing promoter regions, the amount of the enzymes of Table 1 can e.g. be increased.

Furthermore, an alteration of the activity of endogenous genes can be achieved by targeted mutagenesis of the endogenous gene copies.

An alteration of the endogenous genes coding for the enzymes of e.g. Table 1 can also be achieved by influencing the post-translational modifications of the enzymes. This can happen e.g. by regulating the activity of enzymes like kinases or phosphatases involved in the post-translational modification of the enzymes by means of corresponding measures like overexpression or gene silencing.

In another embodiment, an enzyme may be improved in efficiency, or its allosteric control region destroyed such that feedback inhibition of production of the compound is prevented. Similarly, a degradative enzyme may be deleted or modified by substitution, deletion, or addition such that its degradative activity is lessened for the desired enzyme of Table 1 without impairing the viability of the cell. In each case, the overall yield, rate of production or amount of methionine be increased.

It is also possible that such alterations in the proteins of e.g. Table 1 may improve the production of other fine chemicals such as other sulfur containing compounds like cysteine or glutathione, other amino acids, vitamins, cofactors, nutraceuticals, nucleic acids, nucleosides, and trehalose. Metabolism of any one compound can be intertwined with other biosynthetic and degradative pathways within the cell, and necessary cofactors, intermediates, or substrates in one pathway may be supplied or limited by another such pathway. Therefore, by modulating the activity of one or more of the proteins of Table 1, the amount, efficiency and rate of other fine chemicals besides methionine may be positively impacted.

These aforementioned strategies for increasing or introducing the amount and/or activity of the enzymes of Table 1 are not meant to be limiting; variations on these strategies will be readily apparent to one of ordinary skill in the art.

Reducing the Amount and/or Activity of Enzymes

It has been set out above that it may be preferred to use starting organism which have already been optimized for methionine production. In C. glutamicum one may, for example, downregulate the activity of metQ.

For reducing the amount and/or activity of enzymes, various strategies are available.

The expression of endogenous enzymes such as those of Table 1 can e.g. be regulated via the expression of aptamers specifically binding to the promoter sequences of the genes. Depending on the aptamer binding to stimulating or repressing promoter regions, the amount and thus, in this case, the activity of the enzymes of Table 1 can e.g. be reduced.

Aptamers can also be designed in a way as to specifically bind to the enzymes themselves and to reduce the activity of the enzymes by e.g. binding to the catalytic center of the respective enzymes. The expression of aptamers is usually achieved by vector-based overexpression (see above) and is, as well as the design and the selection of aptamers, well known to the person skilled in the art (Famulok et al., (1999) Curr Top Microbiol Immunol., 243, 123-36).

Furthermore, a decrease of the amount and the activity of the endogenous enzymes of Table 1 can be achieved by means of various experimental measures, which are well known to the person skilled in the art. These measures are usually summarized under the term “gene silencing”. For example, the expression of an endogenous gene can be silenced by transferring an above-mentioned vector, which has a DNA sequence coding for the enzyme or parts thereof in antisense order, to organisms such as C. glutamicum. This is based on the fact that the transcription of such a vector in the cell leads to an RNA, which can hybridize with the mRNA transcribed by the endogenous gene and therefore prevents its translation.

In principle, the antisense strategy can be coupled with a ribozyme method. Ribozymes are catalytically active RNA sequences, which, if coupled to the antisense sequences, cleave the target sequences catalytically (Tanner et al., (1999) FEMS Microbiol Rev. 23 (3), 257-75). This can enhance the efficiency of an antisense strategy.

To create a homologous recombinant microorganism, a vector is prepared which contains at least a portion of gene coding for an enzyme of Table 1 into which a deletion, addition or substitution has been introduced to thereby alter, e.g., functionally disrupt, the endogenous gene.

In one embodiment, the vector is designed such that, upon homologous recombination, the endogenous gene is functionally disrupted (i.e., no longer encodes a functional protein). Alternatively, the vector can be designed such that, upon homologous recombination, the endogenous gene is mutated or otherwise altered but still encodes functional protein, e.g., the upstream regulatory region can be altered to thereby alter the expression of the endogenous enzymes of e.g. Table 1. This approach can have the advantage that expression of an enzyme is not completely abolished, but reduced to the required minimum level. The skilled person knows which vectors can be used to replace or delete endogenous sequences. For. C. glutamicum, such vectors include pK19 and pCLIK int sacB. A specific description for disrupting chromosomal sequences in C. glutamicum is provided below.

Furthermore, gene repression is possible by reducing the amount of transcription factors.

Factors inhibiting the target protein itself can also be introduced into a cell. The protein-binding factors may e.g. be the above-mentioned aptamers (Famulok et al., (1999) Curr Top Microbiol Immunol 243, 123-36).

As further protein-binding factors, the expression of which can cause a reduction of the amount and/or the activity of the enzymes of table 1, enzyme-specific antibodies may be considered. The production of recombinant enzyme-specific antibodies such as single chain antibodies is known in the art. The expression of antibodies is also known from the literature (Fiedler et al., (1997) Immunotechnology 3, 205-216; Maynard and Georgiou (2000) Annu. Rev. Biomed. Eng. 2, 339-76).

The mentioned techniques are well known to the person skilled in the art. Therefore, the skilled also knows the typical size that a nucleic acid constructs used for e.g. antisense methods must have and which complementarity, homology or identity, the respective nucleic acid sequences must have. The terms complementarity, homology, and identity are known to the person skilled in the art.

The term complementarity describes the capability of a nucleic acid molecule to hybridize with another nucleic acid molecule due to hydrogen bonds between two complementary bases. The person skilled in the art knows that two nucleic acid molecules do not have to display a complementarity of 100% in order to be able to hybridize with each other. A nucleic acid sequence, which is to hybridize with another nucleic acid sequence, is preferably at least 30%, at least 40%, at least 50%, at least 60%, preferably at least 70%, particularly preferred at least 80%, also particularly preferred at least 90%, in particular preferred at least 95% and most preferably at least 98 or 100%, respectively, complementary with said other nucleic acid sequence.

The hybridization of an antisense sequence with an endogenous mRNA sequence typically occurs in vivo under cellular conditions or in vitro. According to the present invention, hybridization is carried out in vivo or in vitro under conditions that are stringent enough to ensure a specific hybridization.

Stringent in vitro hybridization conditions are known to the person skilled in the art and can be taken from the literature (see e.g. Sambrook et al., Molecular Cloning, Cold Spring Harbor Press (2001)). The term “specific hybridization” refers to the case wherein a molecule preferentially binds to a certain nucleic acid sequence under stringent conditions, if this nucleic acid sequence is part of a complex mixture of e.g. DNA or RNA molecules.

The term “stringent conditions” therefore refers to conditions, under which a nucleic acid sequence preferentially binds to a target sequence, but not, or at least to a significantly reduced extent, to other sequences.

Stringent conditions are dependent on the circumstances. Longer sequences specifically hybridize at higher temperatures. In general, stringent conditions are chosen in such a way that the hybridization temperature lies about 5° C. below the melting point (Tm) of the specific sequence with a defined ionic strength and a defined pH value. Tm is the temperature (with a defined pH value, a defined ionic strength and a defined nucleic acid concentration), at which 50% of the molecules, which are complementary to a target sequence, hybridize with said target sequence. Typically, stringent conditions comprise salt concentrations between 0.01 and 1.0 M sodium ions (or ions of another salt) and a pH value between 7.0 and 8.3. The temperature is at least 30° C. for short molecules (e.g. for such molecules comprising between 10 and 50 nucleic acids). In addition, stringent conditions can comprise the addition of destabilizing agents like e.g. form amide. Typical hybridization and washing buffers are of the following composition.

| | |
|---|---|
| Pre-hybridization solution: | 0.5% SDS |
| | 5x SSC |
| | 50 mM NaPO₄, pH 6.8 |
| | 0.1% Na-pyrophosphate |
| | 5x Denhardt's reagent |
| | 100 μg/salmon sperm |
| Hybridization solution: | Pre-hybridization solution |
| | 1 × 10⁶ cpm/ml probe (5-10 min 95° C.) |
| 20x SSC: | 3 M NaCl |
| | 0.3 M sodium citrate |
| | ad pH 7 with HCl |
| 50x Denhardt's reagent: | 5 g Ficoll |
| | 5 g polyvinylpyrrolidone |
| | 5 g Bovine Serum Albumin |
| | ad 500 ml A. dest. |

A typical procedure for the hybridization is as follows:

| Optional: | wash Blot 30 min in 1x SSC/0.1% SDS at 65° C. | | |
|---|---|---|---|
| Pre-hybridization: | at least 2 h at 50-55° C. | | |
| Hybridization: | over night at 55-60° C. | | |
| Washing: | 05 min | 2x SSC/0.1% SDS | |
| | Hybridization temperature | | |
| | 30 min | 2x SSC/0.1% SDS | |
| | Hybridization temperature | | |
| | 30 min | 1x SSC/0.1% SDS | |
| | Hybridization temperature | | |
| | 45 min | 0.2x SSC/0.1% SDS | 65° C. |
| | 5 min | 0.1x SSC | room temperature |

For antisense purposes complementarity over sequence lengths of 100 nucleic acids, 80 nucleic acids, 60 nucleic acids, 40 nucleic acids and 20 nucleic acids may suffice. Longer nucleic acid lengths will certainly also suffice. A combined application of the above-mentioned methods is also conceivable.

If, according to the present invention, DNA sequences are used, which are operatively linked in 5′-3′-orientation to a promoter active in the organism, vectors can, in general, be constructed, which, after the transfer to the organism's cells, allow the overexpression of the coding sequence or cause the suppression or competition and blockage of endogenous nucleic acid sequences and the proteins expressed there from, respectively.

The activity of a particular enzyme may also be reduced by over-expressing a non-functional mutant thereof in the organism. Thus, a non-functional mutant which is not able to catalyze the reaction in question, but that is able to bind e.g. the substrate or co-factor, can, by way of over-expression out-compete the endogenous enzyme and therefore inhibit the reaction. Further methods in order to reduce the amount and/or activity of an enzyme in a host cell are well known to the person skilled in the art.

According to the present invention, non-functional enzymes have essentially the same nucleic acid sequences and amino acid sequences, respectively, as functional enzymes and functionally fragments thereof, but have, at some positions, point mutations, insertions or deletions of nucleic acids or amino acids, which have the effect that the non-functional enzyme are not, or only to a very limited extent, capable of catalyzing the respective reaction. These non-functional enzymes may not be intermixed with enzymes that still are capable of catalyzing the respective reaction, but which are not feedback regulated anymore. According to the present invention, the term “non-functional enzyme” does not comprise such proteins having no substantial sequence homology to the respective functional enzymes at the amino acid level and nucleic acid level, respectively. Proteins unable to catalyse the respective reactions and having no substantial sequence homology with the respective enzyme are therefore, by definition, not meant by the term “non-functional enzyme” of the present invention. Non-functional enzymes are, within the scope of the present invention, also referred to as inactivated or inactive enzymes.

Therefore, non-functional enzymes of e.g. Table 1 according to the present invention bearing the above-mentioned point mutations, insertions, and/or deletions are characterized by an substantial sequence homology to the wild type enzymes of e.g. Table 1 according to the present invention or functionally equivalent parts thereof. For determining a substantial sequence homology, the above described identity grades are to applied.

### Vectors and Host Cells

One aspect of the invention pertains to vectors, preferably expression vectors, containing a nucleic acid sequences as mentioned above. As used herein, the term “vector” refers to a nucleic acid molecule capable of transporting another nucleic acid to which it has been linked.

One type of vector is a “plasmid”, which refers to a circular double stranded DNA loop into which additional DNA segments can be ligated. Another type of vector is a viral vector, wherein additional DNA segments can be ligated into the viral genome.

Certain vectors are capable of autonomous replication in a host cell into which they are introduced (e.g., bacterial vectors having a bacterial origin of replication and episomal mammalian vectors). Other vectors are integrated into the genome of a host cell upon introduction into the host cell, and thereby are replicated along with the host genome. Moreover, certain vectors are capable of directing the expression of genes to which they are operatively linked.

Such vectors are referred to herein as “expression vectors”.

In general, expression vectors of utility in recombinant DNA techniques are often in the form of plasmids. In the present specification, “plasmid” and “vector” can be used interchangeably as the plasmid is the most commonly used form of vector. However, the invention is intended to include other forms of expression vectors, such as viral vectors, which serve equivalent functions.

The recombinant expression vectors of the invention may comprise a nucleic acid as mentioned above in a form suitable for expression of the respective nucleic acid in a host cell, which means that the recombinant expression vectors include one or more regulatory sequences, selected on the basis of the host cells to be used for expression, which are operatively linked to the nucleic acid sequence to be expressed.

For the purposes of the present invention, an operative link is understood to be the sequential arrangement of promoter, coding sequence, terminator and, optionally, further regulatory elements in such a way that each of the regulatory elements can fulfill its function, according to its determination, when expressing the coding sequence.

Within a recombinant expression vector, “operably linked” is thus intended to mean that the nucleic acid sequence of interest is linked to the regulatory sequence (s) in a manner which allows for expression of the nucleic acid sequence (e.g., in an in vitro transcription/translation system or in a host cell when the vector is introduced into the host cell). The term “regulatory sequence” is intended to include promoters, repressor binding sites, activator binding sites, enhancers and other expression control elements (e.g., terminators or other elements of mRNA secondary structure). Such regulatory sequences are described, for example, in Goeddel; Gene Expression Technology: Methods in Enzymology 185, Academic Press, San Diego, Calif. (1990). Regulatory sequences include those which direct constitutive expression of a nucleic acid sequence in many types of host cell and those which direct expression of the nucleic acid sequence only in certain host cells. Preferred regulatory sequences are, for example, promoters such as cos-, tac-, trp-, tet-, trp-, tet-, lpp-, lac-, lpp-lac-, lacIq-, T7-, T5-, T3-, gal-, trc-, ara-, SP6-, amy, SP02, phage lambdaP_{R} (also known as λP_{R}), phage lambdaP_{L} (also known as λP_{L}), phage SP01 P₁₅, phage SP01 P₂₆, pSOD, EFTu, EFTs, GroEL, MetZ (last 5 from C. glutamicum), which are used preferably in bacteria. Additional regulatory sequences are, for example, promoters from yeasts and fungi, such as ADC1, MFa, AC, P-60, CYC1, GAPDH, TEF, rp28, ADH, ENO2, promoters from plants such as CaMV/35S, SSU, OCS, lib4, usp, STLS1, B33, nos or ubiquitin- or phaseolin-promoters. It is also possible to use artificial promoters. It will be appreciated by one of ordinary skill in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by the above-mentioned modified nucleic acid sequences.

Expression of proteins in prokaryotes is most often carried out with vectors containing constitutive or inducible promoters directing the expression of either fusion or non-fusion proteins.

Fusion vectors add a number of amino acids to a protein encoded therein, usually to the amino terminus of the recombinant protein but also to the C-terminus or fused within suitable regions in the proteins. Such fusion vectors typically serve three 4 purposes: 1) to increase expression of recombinant protein; 2) to increase the solubility of the recombinant protein; and 3) to aid in the purification of the recombinant protein by acting as a ligand in affinity purification 4) to provide a “tag” for later detection of the protein. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase.

Typical fusion expression vectors include pGEX (Pharmacia Biotech Inc; Smith, D. B. and Johnson, K. S. (1988) Gene 67: 31-40), pMAL (New England Biolabs, Beverly, Mass.) and pRIT5 (Pharmacia, Piscataway, N.J.) which fuse glutathione S-transferase (GST), maltose E binding protein, or protein A, respectively.

Examples of suitable inducible non-fusion expression vectors for Coryneform bacteria include pHM1519, pBL1, pSA77 or pAJ667 (Pouwels et al., eds. (1985) Cloning Vectors. Elsevier: New York IBSN 0 444 904018). Examples of suitable C. glutamicum and E. coli shuttle vectors are e.g. pK19, pClik5aMCS pCLIKint sacB or can be found in Eikmanns et al (Gene. (1991) 102, 93-8) and in the following publications and patent applications (Schafer A, et al. J Bacteriol. 1994 176: 7309-7319, Bott, M. and Eggeling, L., eds. Handbook of Corynebacterium glutamicum. CRC Press LLC, Boca Raton, Fla. WO2006069711, WO2006069711). For other suitable expression systems for both prokaryotic and eukaryotic cells see chapters 16 and 17 of Sambrook, J. et al. Molecular Cloning: A Laboratory Manual. 3rd ed., Cold Spring Harbor Laboratory, Cold Spring Harbor Laboratory Press, Cold Spring Harbor, N.Y., 2003.

Vector DNA can be introduced into prokaryotic via conventional transformation or transfection techniques. As used herein, the terms “transformation” and “transfection”, “conjugation” and “transduction” are intended to refer to a variety of art-recognized techniques for introducing foreign nucleic acid (e.g., linear DNA or RNA (e.g., a linearized vector or a gene construct alone without a vector) or nucleic acid in the form of a vector (e.g., a plasmid, phage, phasmid, phagemid, transposon or other DNA into a host cell, including calcium phosphate or calcium chloride co-precipitation, DEAE-dextran-mediated transfection, lipofection, natural competence, chemical-mediated transfer, or electroporation. Suitable methods for transforming or transfecting host cells can be found in Sambrook, et al. (Molecular Cloning: A Laboratory Manual. 3rd ed., Cold Spring Harbor Laboratory, Cold Spring Harbor Laboratory Press, Cold Spring Harbor, N.Y., 2003), and other laboratory manuals.

In order to identify and select these integrants, a gene that encodes a selectable marker (e.g., resistance to antibiotics) is generally introduced into the host cells along with the gene of interest. Preferred selectable markers include those which confer resistance to drugs, such as G418, hygromycin, kanamycine, tetracycline, chloramphenicol, ampicillin and methotrexate. Nucleic acid encoding a selectable marker can be introduced into a host cell on the same vector as that encoding the above-mentioned modified nucleic acid sequences or can be introduced on a separate vector. Cells stably transfected with the introduced nucleic acid can be identified by drug selection (e.g., cells that have incorporated the selectable marker gene will survive, while the other cells die).

In another embodiment, recombinant microorganisms can be produced which contain selected systems which allow for regulated expression of the introduced gene. For example, inclusion of one of the above-mentioned nucleic acid sequences on a vector placing it under control of the lac operon permits expression of the gene only in the presence of IPTG. Such regulatory systems are well known in the art.

Another aspect of the invention pertains to organisms or host cells into which a recombinant expression vector of the invention has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

Growth of C. glutamicum-Media and Culture Conditions

A general teaching will be given below as to the cultivation of C. glutamicum. Adaptions will be obvious to the skilled person Corresponding information may be retrieved from standard textbooks for cultivation of E. coli.

Genetically modified Corynebacteria are typically cultured in synthetic or natural growth media. A number of different growth media for Corynebacteria are both well-known and readily available (Lieb et al. (1989) Appl. Microbiol. Biotechnol., 32: 205-210; von der Osten et al. (1998) Biotechnology Letters, 11: 11-16; U.S. Pat. No. DE 4,120,867; Liebl (1992) “The Genus Corynebacterium, in: The Procaryotes, Volume II, Balows, A. et al., eds. Springer-Verlag).

These media consist of one or more carbon sources, nitrogen sources, inorganic salts, vitamins and trace elements. Preferred carbon sources are sugars, such as mono-, di-, or polysaccharides. For example, glucose, fructose, mannose, galactose, ribose, sorbose, ribose, lactose, maltose, sucrose, raffinose, starch or cellulose serve as very good carbon sources.

It is also possible to supply sugar to the media via complex compounds such as molasses or other by-products from sugar refinement. It can also be advantageous to supply mixtures of different carbon sources. Other possible carbon sources are alcohols and organic acids, such as methanol, ethanol, acetic acid or lactic acid. Nitrogen sources are usually organic or inorganic nitrogen compounds, or materials which contain these compounds. Exemplary nitrogen sources include ammonia gas or ammonia salts, such as NH₄Cl or (NH₄)₂S0₄, NH₄OH, nitrates, urea, amino acids or complex nitrogen sources like corn steep liquor, soy bean flour, soy bean protein, yeast extract, meat extract and others.

Inorganic salt compounds which may be included in the media include the chloride-, phosphorous- or sulfate-salts of calcium, magnesium, sodium, cobalt, molybdenum, potassium, manganese, zinc, copper and iron. Chelating compounds can be added to the medium to keep the metal ions in solution. Particularly useful chelating compounds include dihydroxyphenols, like catechol or protocatechuate, or organic acids, such as citric acid. It is typical for the media to also contain other growth factors, such as vitamins or growth promoters, examples of which include biotin, riboflavin, thiamine, folic acid, nicotinic acid, pantothenate and pyridoxine. Growth factors and salts frequently originate from complex media components such as yeast extract, molasses, corn steep liquor and others. The exact composition of the media compounds depends strongly on the immediate experiment and is individually decided for each specific case. Information about media optimization is available in the textbook “Applied Microbiol. Physiology, A Practical Approach (Eds. P. M. Rhodes, P. F. Stanbury, IRL Press (1997) pp. 53-73, ISBN 0 19 963577 3). It is also possible to select growth media from commercial suppliers, like standard 1 (Merck) or BHI (grain heart infusion, DIFCO) or others.

All medium components should be sterilized, either by heat (20 minutes at 1.5 bar and 121° C.) or by sterile filtration. The components can either be sterilized together or, if necessary, separately.

All media components may be present at the beginning of growth, or they can optionally be added continuously or batch wise. Culture conditions are defined separately for each experiment.

The temperature should be in a range between 15° C. and 45° C. The temperature can be kept constant or can be altered during the experiment. The pH of the medium may be in the range of 5 to 8.5, preferably around 7.0, and can be maintained by the addition of buffers to the media. An exemplary buffer for this purpose is a potassium phosphate buffer. Synthetic buffers such as MOPS, HEPES, ACES and others can alternatively or simultaneously be used. It is also possible to maintain a constant culture pH through the addition of NaOH or NH₄OH during growth. If complex medium components such as yeast extract are utilized, the necessity for additional buffers may be reduced, due to the fact that many complex compounds have high buffer capacities. If a fermentor is utilized for culturing the microorganisms, the pH can also be controlled using gaseous ammonia.

The incubation time is usually in a range from several hours to several days. This time is selected in order to permit the maximal amount of product to accumulate in the broth. The disclosed growth experiments can be carried out in a variety of vessels, such as microtiter plates, glass tubes, glass flasks or glass or metal fermentors of different sizes. For screening a large number of clones, the microorganisms should be cultured in microtiter plates, glass tubes or shake flasks, either with or without baffles. Preferably 100 ml or 250 ml shake flasks are used, filled with 10% (by volume) of the required growth medium. The flasks should be shaken on a rotary shaker (amplitude 25 mm) using a speed-range of 100-300′ rpm. Evaporation losses can be diminished by the maintenance of a humid atmosphere; alternatively, a mathematical correction for evaporation losses should be performed.

If genetically modified clones are tested, an unmodified control clone or a control clone containing the basic plasmid without any insert should also be tested. The medium is inoculated to an OD600 of 0.5-1.5 using cells grown on agar plates, such as CM plates (10 g/l glucose, 2.5 g/l NaCl, 2 g/l urea, 10 g/l polypeptone, 5 g/l yeast extract, 5 g/l meat extract, 2 g/l urea, 10 g/l polypeptone, 5 g/l yeast extract, 5 g/l meat extract, 22 g/l agar, pH 6.8 with 2M NaOH) that had been incubated at 30 C. Inoculation of the media is accomplished by either introduction of a saline suspension of C. glutamicum cells from CM plates or addition of a liquid preculture of this bacterium. Other incubation methods can be taken from WO2007012078.

### General Methods

Protocols for general methods can be found in Handbook on Corynebacterium glutamicum, (2005) eds.: L. Eggeling, M. Bott., Boca Raton, CRC Press, at Martin et al. (Biotechnology (1987) 5, 137-146), Guerrero et al. (Gene (1994), 138, 35-41), Tsuchiya und Morinaga (Biotechnology (1988), 6, 428-430), Eikmanns et al. (Gene (1991), 102, 93-98), EP 0 472 869, U.S. Pat. No. 4,601,893, Schwarzer and Pühler (Biotechnology (1991), 9, 84-87, Reinscheid et al. (Applied and Environmental Microbiology (1994), 60, 126-132), LaBarre et al. (Journal of Bacteriology (1993), 175, 1001-1007), WO 96/15246, Malumbres et al. (Gene (1993), 134, 15-24), in JP-A-10-229891, at Jensen und Hammer (Biotechnology and Bioengineering (1998), 58, 191-195), Makrides (Microbiological Reviews (1996), 60, 512-538) in WO2006069711, in WO2007012078 and in well known textbooks of genetic and molecular biology.

### Strains, Media and Plasmids

Strains can be taken e.g. from the following list:

Corynebacterium glutamicum ATCC 13032,
Corynebacterium acetoglutamicum ATCC 15806,
Corynebacterium acetoacidophilum ATCC 13870,
Corynebacterium thermoaminogenes FERM BP-1539,
Corynebacterium melassecola ATCC 17965,
Brevibacterium flavum ATCC 14067,
Brevibacterium lactofermentum ATCC 13869, and
Brevibacterium divaricatum ATCC 14020 or strains which have been derived therefrom such as Corynebacterium glutamicum KFCC10065, DSM 17322 or
Corynebacterium glutamicum ATCC21608

### Recombinant DNA Technology

Protocols can be found in: Sambrook, J., Fritsch, E. F., and Maniatis, T., in Molecular Cloning: A Laboratory Manual, 3^{rd} edition (2001) Cold Spring Harbor Laboratory Press, NY, Vol. 1, 2, 3, and Handbook on Corynebacterium glutamicum (2005) eds. L. Eggeling, M. Bott., Boca Raton, CRC Press.

### Quantification of Amino Acids and Methionine Intermediates.

The analysis is done by HPLC (Agilent 1100, Agilent, Waldbronn, Germany) with a guard cartridge and a Synergi 4 μm column (MAX-RP 80 Å, 150*4.6 mm) (Phenomenex, Aschaffenburg, Germany). Prior to injection the analytes are derivatized using o-phthaldialdehyde (OPA) and mercaptoethanol as reducing agent (2-MCE). Additionally sulfhydryl groups are blocked with iodoacetic acid. Separation is carried out at a flow rate of 1 ml/min using 40 mM NaH₂PO₄ (eluent A, pH=7.8, adjusted with NaOH) as polar and a methanol water mixture (100/1) as non-polar phase (eluent B). The following gradient is applied: Start 0% B; 39 min 39% B; 70 min 64% B; 100% B for 3.5 min; 2 min 0% B for equilibration. Derivatization at room temperature is automated as described below. Initially 0.5 μl of 0.5% 2-MCE in bicine (0.5M, pH 8.5) are mixed with 0.5 μl cell extract. Subsequently 1.5 μl of 50 mg/ml iodoacetic acid in bicine (0.5M, pH 8.5) are added, followed by addition of 2.5 μl bicine buffer (0.5M, pH 8.5). Derivatization is done by adding 0.5 μl of 10 mg/ml OPA reagent dissolved in Jan. 45, 1954 v/v/v of 2-MCE/MeOH/bicine (0.5M, pH 8.5). Finally the mixture is diluted with 32 μl H₂O. Between each of the above pipetting steps there is a waiting time of 1 min. A total volume of 37.5 μl is then injected onto the column. Note, that the analytical results can be significantly improved, if the auto sampler needle is periodically cleaned during (e.g. within waiting time) and after sample preparation. Detection is performed by a fluorescence detector (340 nm excitation, emission 450 nm, Agilent, Waldbronn, Germany). For quantification α-amino butyric acid (ABA) was is as internal standard

### DEFINITION OF RECOMBINATION PROTOCOL

In the following it will be described how a strain of C. glutamicum with increased efficiency of methionine production can be constructed implementing the findings of the above predictions. Before the construction of the strain is described, a definition of a recombination event/protocol is given that will be used in the following.

“Campbell in,” as used herein, refers to a transformant of an original host cell in which an entire circular double stranded DNA molecule (for example a plasmid being based on pCLIK int sacB or pK19 has integrated into a chromosome by a single homologous recombination event (a cross-in event), and that effectively results in the insertion of a linearized version of said circular DNA molecule into a first DNA sequence of the chromosome that is homologous to a first DNA sequence of the said circular DNA molecule. “Campbelled in” refers to the linearized DNA sequence that has been integrated into the chromosome of a “Campbell in” transformant. A “Campbell in” contains a duplication of the first homologous DNA sequence, each copy of which includes and surrounds a copy of the homologous recombination crossover point. The name comes from Professor Alan Campbell, who first proposed this kind of recombination.

“Campbell out,” as used herein, refers to a cell descending from a “Campbell in” transformant, in which a second homologous recombination event (a cross out event) has occurred between a second DNA sequence that is contained on the linearized inserted DNA of the “Campbelled in” DNA, and a second DNA sequence of chromosomal origin, which is homologous to the second DNA sequence of said linearized insert, the second recombination event resulting in the deletion (jettisoning) of a portion of the integrated DNA sequence, but, importantly, also resulting in a portion (this can be as little as a single base) of the integrated Campbelled in DNA remaining in the chromosome, such that compared to the original host cell, the “Campbell out” cell contains one or more intentional changes in the chromosome (for example, a single base substitution, multiple base substitutions, insertion of a heterologous gene or DNA sequence, insertion of an additional copy or copies of a homologous gene or a modified homologous gene, or insertion of a DNA sequence comprising more than one of these aforementioned examples listed above).

A “Campbell out” cell or strain is usually, but not necessarily, obtained by a counter-selection against a gene that is contained in a portion (the portion that is desired to be jettisoned) of the “Campbelled in” DNA sequence, for example the Bacillus subtilis sacB gene, which is lethal when expressed in a cell that is grown in the presence of about 5% to 10% sucrose. Either with or without a counter-selection, a desired “Campbell out” cell can be obtained or identified by screening for the desired cell, using any screenable phenotype, such as, but not limited to, colony morphology, colony color, presence or absence of antibiotic resistance, presence or absence of a given DNA sequence by polymerase chain reaction, presence or absence of an auxotrophy, presence or absence of an enzyme, colony nucleic acid hybridization, antibody screening, etc. The term “Campbell in” and “Campbell out” can also be used as verbs in various tenses to refer to the method or process described above.

It is understood that the homologous recombination events that leads to a “Campbell in” or “Campbell out” can occur over a range of DNA bases within the homologous DNA sequence, and since the homologous sequences will be identical to each other for at least part of this range, it is not usually possible to specify exactly where the crossover event occurred. In other words, it is not possible to specify precisely which sequence was originally from the inserted DNA, and which was originally from the chromosomal DNA. Moreover, the first homologous DNA sequence and the second homologous DNA sequence are usually separated by a region of partial non-homology, and it is this region of non-homology that remains deposited in a chromosome of the “Campbell out” cell.

For practicality, in C. glutamicum, typical first and second homologous DNA sequence are at least about 200 base pairs in length, and can be up to several thousand base pairs in length, however, the procedure can be made to work with shorter or longer sequences. For example, a length for the first and second homologous sequences can range from about 500 to 2000 bases, and the obtaining of a “Campbell out” from a “Campbell in” is facilitated by arranging the first and second homologous sequences to be approximately the same length, preferably with a difference of less than 200 base pairs and most preferably with the shorter of the two being at least 70% of the length of the longer in base pairs. A description of the Campbell in and out method can be taken from WO2007012078.

### Examples

The following experiments demonstrate how overexpression of C. glutamicum transketolase leads to increased methionine production. These examples are however in no way meant to limit the invention in any way.

### Shake Flask Experiments and HPLC Assay

Shake flasks experiments, with the standard Molasses Medium, were performed with strains in duplicate or quadruplicate. Molasses Medium contained in one liter of medium: 40 g glucose; 60 g molasses; 20 g (NH₄)₂SO₄; 0.4 g MgSO₄*7H₂O; 0.6 g KH₂PO₄; 10 g yeast extract (DIFCO); 5 ml of 400 mM threonine; 2 mgFeSO₄.7H₂O; 2 mg of MnSO₄.H₂O; and 50 g CaCO₃ (Riedel-de Haen), with the volume made up with ddH₂O. The pH was adjusted to 7.8 with 20% NH₄OH, 20 ml of continuously stirred medium (in order to keep CaCO₃ suspended) was added to 250 ml baffled Bellco shake flasks and the flasks were autoclaved for 20 min. Subsequent to autoclaving, 4 ml of “4B solution” was added per liter of the base medium (or 80 μl/flask). The “4B solution” contained per liter: 0.25 g of thiamine hydrochloride (vitamin B1), 50 mg of cyanocobalamin (vitamin B12), 25 mg biotin, 1.25 g pyridoxine hydrochloride (vitamin B6) and was buffered with 12.5 mM KPO₄, pH 7.0 to dissolve the biotin, and was filter sterilized. In some experiments, the final concentration of vitamin B12 was varied to be more or less than the standard concentration. Cultures were grown in baffled flasks covered with Bioshield paper secured by rubber bands for 48 hours at 28° C. or 30° C. and at 200 or 300 rpm in a New Brunswick Scientific floor shaker. Samples were taken at 24 hours and/or 48 hours. Cells were removed by centrifugation followed by dilution of the supernatant with an equal volume of 60% acetonitrile and then membrane filtration of the solution using Centricon 0.45 μm spin columns. The filtrates were assayed using HPLC for the concentrations of methionine, glycine plus homoserine, O-acetylhomoserine, threonine, isoleucine, lysine, and other indicated amino acids.

For the HPLC assay, filtered supernatants were diluted 1:100 with 0.45 μm filtered 1 mM Na₂EDTA and 1 μl of the solution was derivatized with OPA reagent (AGILENT) in Borate buffer (80 mM NaBO₃, 2.5 mM EDTA, pH 10.2) and injected onto a 200×4.1 mm Hypersil 5μ AA-ODS column run on an Agilent 1100 series HPLC equipped with a G1321A fluorescence detector (AGILENT). The excitation wavelength was 338 nm and the monitored emission wavelength was 425 nm. Amino acid standard solutions were chromatographed and used to determine the retention times and standard peak areas for the various amino acids. Chem Station, the accompanying software package provided by Agilent, was used for instrument control, data acquisition and data manipulation. The hardware was an HP Pentium 4 computer that supports Microsoft Windows NT 4.0 updated with a Microsoft Service Pack (SP6a).

### Experiment 1—Generation of the M2014 Strain

C. glutamicum strain ATCC 13032 was transformed with DNA A (also referred to as pH273) (SEQ ID NO: 13) and “Campbelled in” to yield a “Campbell in” strain. The “Campbell in” strain was then “Campbelled out” to yield a “Campbell out” strain, M440, which contains a gene encoding a feedback resistant homoserine dehydrogenase enzyme (hom^{fbr}). The resultant homoserine dehydrogenase protein included an amino acid change where S393 was changed to F393 (referred to as Hsdh S393F).

The strain M440 was subsequently transformed with DNA B (also referred to as pH373) (SEQ ID NO: 14) to yield a “Campbell in” strain. The “Campbell in” strain were then “Campbelled out” to yield a “Campbell out” strain, M603, which contains a gene encoding a feedback resistant aspartate kinase enzyme (Ask^{fbr}) (encoded by lysC). In the resulting aspartate kinase protein, T311 was changed to I311 (referred to as LysC T311I).

It was found that the strain M603 produced about 17.4 mM lysine, while the ATCC13032 strain produced no measurable amount of lysine. Additionally, the M603 strain produced about 0.5 mM homoserine, compared to no measurable amount produced by the ATCC13032 strain, as summarized in Table 2.

TABLE 2
Amounts of homoserine, O-acetylhomoserine, methionine and
lysine produced by strains ATCC13032 and M603

| | | O-acetyl | | |
|---|---|---|---|---|
| | Homoserine | homoserine | Methionine | Lysine |
| Strain | (mM) | (mM) | (mM) | (mM) |
| ATCC13032 | 0.0 | 0.4 | 0.0 | 0.0 |
| M603 | 0.5 | 0.7 | 0.0 | 17.4 |

The strain M603 was transformed with DNA C (also referred to as pH304) (SEQ ID NO:15) to yield a “Campbell in” strain, which was then “Campbelled out” to yield a “Campbell out” strain, M690. The M690 strain contained a PgroES promoter upstream of the metH gene (referred to as P₄₉₇ metH). The sequence of the P₄₉₇ promoter is depicted in SEQ ID NO: 10. The M690 strain produced about 77.2 mM lysine and about 41.6 mM homoserine, as shown below in Table 3.

TABLE 3
Amounts of homoserine, O-acetyl homoserine, methionine and
lysine produced by the strains M603 and M690

| | | O-acetyl | | |
|---|---|---|---|---|
| | Homoserine | homoserine | Methionine | Lysine |
| Strain | (mM) | (mM) | (mM) | (mM) |
| M603 | 0.5 | 0.7 | 0.0 | 17.4 |
| M690 | 41.6 | 0.0 | 0.0 | 77.2 |

The M690 strain was subsequently mutagenized as follows: an overnight culture of M603, grown in BHI medium (BECTON DICKINSON), was washed in 50 mM citrate buffer pH 5.5, treated for 20 min at 30° C. with N-methyl-N-nitrosoguanidine (10 mg/ml in 50 mM citrate pH 5.5). After treatment, the cells were again washed in 50 mM citrate buffer pH 5.5 and plated on a medium containing the following ingredients: (all mentioned amounts are calculated for 500 ml medium) 10 g (NH₄)₂SO₄; 0.5 g KH₂PO₄; 0.5 g K₂HPO₄; 0.125 g MgSO₄*7H₂O; 21 g MOPS; 50 mg CaCl₂; 15 mg protocatechuic acid; 0.5 mg biotin; 1 mg thiamine; and 5 g/l D,L-ethionine (SIGMA CHEMICALS, CATALOG #E5139), adjusted to pH 7.0 with KOH. In addition the medium contained 0.5 ml of a trace metal solution composed of: 10 g/l FeSO₄*7H₂O; 1 g/l MnSO₄*H₂O; 0.1 g/l ZnSO₄*7H₂O; 0.02 g/l CuSO₄; and 0.002 g/l NiCl₂*6H₂O, all dissolved in 0.1 M HCl. The final medium was sterilized by filtration and to the medium, 40 mls of sterile 50% glucose solution (40 ml) and sterile agar to a final concentration of 1.5% were added. The final agar containing medium was poured to agar plates and was labeled as minimal-ethionine medium. The mutagenized strains were spread on the plates (minimal-ethionine) and incubated for 3-7 days at 30° C. Clones that grew on the medium were isolated and restreaked on the same minimal-ethionine medium. Several clones were selected for methionine production analysis.

Methionine production was analyzed as follows. Strains were grown on CM-agar medium for two days at 30° C., which contained: 10 g/l D-glucose, 2.5 g/l NaCl; 2 g/l urea; 10 g/l Bacto Peptone (DIFCO); 5 g/l Yeast Extract (DIFCO); 5 g/l Beef Extract (DIFCO); 22 g/l Agar (DIFCO); and which was autoclaved for 20 min at about 121° C.

After the strains were grown, cells were scraped off and resuspended in 0.15 M NaCl. For the main culture, a suspension of scraped cells was added at a starting OD of 600 nm to about 1.5 to 10 ml of Medium II (see below) together with 0.5 g solid and autoclaved CaCO₃ (RIEDEL DE HAEN) and the cells were incubated in a 100 ml shake flask without baffles for 72 h on a orbital shaking platform at about 200 rpm at 30° C. Medium II contained: 40 g/l sucrose; 60 g/l total sugar from molasses (calculated for the sugar content); 10 g/l (NH₄)₂SO₄; 0.4 g/l MgSO₄*7H₂O; 0.6 g/l KH₂PO₄; 0.3 mg/l thiamine*HCl; 1 mg/l biotin; 2 mg/l FeSO₄; and 2 mg/l MnSO₄. The medium was adjusted to pH 7.8 with NH₄OH and autoclaved at about 121° C. for about 20 min). After autoclaving and cooling, vitamin B₁₂ (cyanocobalamine) (SIGMA CHEMICALS) was added from a filter sterile stock solution (200 μg/ml) to a final concentration of 100 μg/l.

Samples were taken from the medium and assayed for amino acid content. Amino acids produced, including methionine, were determined using the Agilent amino acid method on an Agilent 1100 Series LC System HPLC. (AGILENT). A pre-column derivatization of the sample with ortho-phthalaldehyde allowed the quantification of produced amino acids after separation on a Hypersil AA-column (AGILENT).

Clones that showed a methionine titer that was at least twice that in M690 were isolated. One such clone, used in further experiments, was named M1197 and was deposited on May 18, 2005, at the DSMZ strain collection as strain number DSM 17322. Amino acid production by this strain was compared to that by the strain M690, as summarized below in Table 4.

TABLE 4
Amounts of homoserine, O-acetylhomoserine, methionine and
lysine produced by strains M690 and M1197

| | | O-acetyl- | | |
|---|---|---|---|---|
| | Homoserine | homoserine | Methionine | Lysine |
| Strain | (mM) | (mM) | (mM) | (mM) |
| M690 | 41.6 | 0.0 | 0.0 | 77.2 |
| M1179 | 26.4 | 1.9 | 0.7 | 79.2 |

The strain M1197 was transformed with DNA F (also referred to as pH399, SEQ ID NO: 16) to yield a “Campbell in” strain, which was subsequently “Campbelled out” to yield strain M1494. This strain contains a mutation in the gene for the homoserine kinase, which results in an amino acid change in the resulting homoserine kinase enzyme from T190 to A190 (referred to as HskT190A). Amino acid production by the strain M1494 was compared to the production by strain M197, as summarized below in Table 5.

TABLE 5
Amounts of homoserine, O-acetylhomoserine, methionine and
lysine produced by strains M1197 and M1494

| | | O-acetyl- | | |
|---|---|---|---|---|
| | Homoserine | homoserine | Methionine | Lysine |
| Strain | (mM) | (mM) | (mM) | (mM) |
| M1197 | 26.4 | 1.9 | 0.7 | 79.2 |
| M1494 | 18.3 | 0.2 | 2.5 | 50.1 |

The strain M1494 was transformed with DNA D (also referred to as pH484, SEQ ID NO:17) to yield a “Campbell in” strain, which was subsequently “Campbelled out” to yield the M1990 strain. The M1990 strain overexpresses a metY allele using both a groES-promoter and an EFTU (elongation factor Tu)-promoter (referred to as P₄₉₇ P₁₂₈₄ metY). The sequence of P₄₉₇P₁₂₈₄ promoter is set forth in SEQ ID NO:18. Amino acid production by the strain M1494 was compared to the production by strain M1990, as summarized below in Table 6.

TABLE 6
Amounts of homoserine, O-acetylhomoserine, methionine and
lysine produced by strains M1494 and M1990

| | | O-acetyl- | | |
|---|---|---|---|---|
| | Homoserine | homoserine | Methionine | Lysine |
| Strain | (mM) | (mM) | (mM) | (mM) |
| M1494 | 18.3 | 0.2 | 2.5 | 50.1 |
| M1990 | 18.2 | 0.3 | 5.6 | 48.9 |

The strain M1990 was transformed with DNA E (also referred to as pH 491, SEQ ID NO: 19) to yield a “Campbell in” strain, which was then “Campbelled out” to yield a “Campbell out” strain M2014. The M2014 strain overexpresses a metA allele using a superoxide dismutase promoter (referred to as P₃₁₁₉ metA). The sequence of P₃₁₁₉ promoter is set forth in SEQ ID NO: 9. Amino acid production by the strain M2014 was compared to the production by strain M11990, as summarized below in Table 7.

TABLE 7
Amounts of homoserine, O-acetylhomoserine, methionine and
lysine produced by strains M1494 and M1990

| | | O-acetyl- | | |
|---|---|---|---|---|
| | Homoserine | homoserine | Methionine | Lysine |
| Strain | (mM) | (mM) | (mM) | (mM) |
| M1990 | 18.2 | 0.3 | 5.6 | 48.9 |
| M2014 | 12.3 | 1.2 | 5.7 | 49.2 |

Experiment 2—Deletion of mcbR from M2014

Plasmid pH429 containing an RXA00655 deletion, (SEQ ID No. 20) was used to introduce the mcbR deletion into C. glutamicum via integration and excision (see WO 2004/050694 A1).

Plasmid pH429 was transformed into the M2014 strain with selection for kanamycin resistance (Campbell in). Using sacB counter-selection, kanamycin-sensitive derivatives of the transformed strain were isolated which presumably had lost the integrated plasmid by excision (Campbell out). The transformed strain produced kanamycin-sensitive derivatives that made small colonies and larger colonies. Colonies of both sizes were screened by PCR to detect the presence of mcbR deletion. None of the larger colonies contained the deletion, whereas 60-70% of the smaller colonies contained the expected mcbR deletion.

When an original isolate was streaked for single colonies on BHI plates, a mixture of tiny and small colonies appeared. When the tiny colonies were restreaked on BHI, once again a mixture of tiny and small colonies appeared. When the small colonies were restreaked on BHI, the colony size was usually small and uniform. Two small single colony isolates, called OM403-4 and OM403-8, were selected for further study.

Shake flask experiments (Table 8) showed that OM403-8 produced at least twice the amount of methionine as the parent M2014. This strain also produced less than one-fifth the amount of lysine as M2014, suggesting a diversion of the carbon flux from aspartate semialdehyde towards homoserine. A third striking difference was a greater than 10-fold increase in the accumulation of isoleucine by OM403 relative to M2014. Cultures were grown for 48 hours in standard molasses medium.

TABLE 8
Amino acid production by isolates of the OM403 strain
in shake flask cultures inoculated with freshly grown cells

| | Colony | Deletion | Met | Lys | Hse + Gly | Ile |
|---|---|---|---|---|---|---|
| Strain | size | ΔmcbR | (g/l) | (g/l) | (g/l) | (g/l) |
| M2014 | Large | none | 0.2 | 2.4 | 0.3 | 0.04 |
| | | | 0.2 | 2.5 | 0.3 | 0.03 |
| | | | 0.2 | 2.4 | 0.3 | 0.03 |
| | | | 0.4 | 3.1 | 0.4 | 0.03 |
| OM403-8 | Small | ΔRXA0655 | 1.0 | 0.3 | 0.8 | 0.8 |
| | | | 1.0 | 0.3 | 0.8 | 0.8 |
| | | | 0.9 | 0.3 | 0.8 | 0.8 |
| | | | 1.0 | 0.3 | 0.8 | 0.6 |

Also as shown in Table 9, there was a greater than 15-fold decrease in the accumulation of O-acetylhomoserine by OM403 relative to M2014. The most likely explanation for this result is that most of the O-acetylhomoserine that accumulates in M2014 is being converted to methionine, homocysteine, and isoleucine in OM403. Cultures were grown for 48 hours in standard molasses medium.

TABLE 9
Amino acid production by two isolates of OM403 in shake
flask cultures inoculated with freshly grown cells.

| | Deletion | Met | OAc-Hse | Ile |
|---|---|---|---|---|
| Strain | ΔmcbR | (g/l) | (g/l) | (g/l) |
| M2014 | None | 0.4 | 3.4 | 0.1 |
| | | 0.4 | 3.2 | 0.1 |
| OM403-4 | ΔRXA0655 | 1.7 | 0.2 | 0.3 |
| | | 1.5 | 0.1 | 0.3 |
| OM403-8 | ΔRXA0655 | 2.2 | <0.05 | 0.6 |
| | | 2.5 | <0.05 | 0.6 |

Experiment 3—Decreasing metQ Expression

In order to decrease the import of methionine in OM403-8, the promoter and 5′ portion of the metQ gene were deleted. The metQ gene encodes a subunit of a methionine import complex that is required for the complex to function. This was accomplished using the standard Campbelling in and Campbelling out technique with plasmid pH449 (SEQ ID NO: 21). OM403-8 and OM456-2 were assayed for methionine production in shake flask assays. The results (Table 10) show that OM456-2 produced more methionine than OM403-8. Cultures were grown for 48 hours in standard molasses medium.

TABLE 10
Shake flask assays of OM456-2

| | | [Met] | [Lys] | [Gly/Hse] | [OAcHS] | [Ile] |
|---|---|---|---|---|---|---|
| Strain | vector | (g/l) | (g/l) | (g/l) | (g/l) | (g/l) |
| OM403-8 | none | 4.0 | 0.8 | 2.2 | 0.4 | 1.9 |
| | | 3.9 | 0.6 | 2.2 | 0.4 | 1.9 |
| OM456-2 | none | 4.2 | 0.4 | 2.3 | 0.4 | 2.3 |
| | | 4.3 | 0.5 | 2.4 | 0.4 | 2.3 |

### Experiment 4—Construction of OM469

A strain referred to as OM469 was constructed which included both deletion of metQ and overexpression of metF by replacing the metF promoter with the phage lambda P_{R} promoter in OM456-2. This was accomplished using the standard Campbelling in and Campbelling out technique with plasmid pOM427 (SEQ ID NO 22). Four isolates of OM469 were assayed for methionine production in shake flask culture assays where they all produced more methionine than OM456-2, as shown in Table 11. Cultures were grown for 48 hours in standard molasses medium containing 2 mM threonine.

TABLE 11
Shake flask assays of OM469, a derivative of OM456-2 containing
the phage lambda P_{R} promoter in place of the metF promoter.

| | | metF | | | | [Gly/ | | |
|---|---|---|---|---|---|---|---|---|
| | | pro- | | [Met] | [Lys] | Hse] | [OAcHS] | [Ile] |
| Strain | | moter | MetQ | (g/l) | (g/l) | (g/l) | (g/l) | (g/l) |
| OM428-2 | | λP_{R} | native | 4.5 | 0.5 | 2.6 | 0.4 | 2.6 |
| | | | | 4.6 | 0.4 | 2.6 | 0.3 | 2.5 |
| OM456-2 | | Native | [symbol] metQ | 4.2 | 0.4 | 2.4 | 0.3 | 2.5 |
| | | | | 4.2 | 0.5 | 2.4 | 0.3 | 2.5 |
| OM469 | 1 | λP_{R} | [symbol] metQ | 5.0 | 0.5 | 2.7 | 0.4 | 3.1 |
| | 2 | | | 4.9 | 0.5 | 2.7 | 0.4 | 2.8 |
| | 3 | | | 4.8 | 0.4 | 2.6 | 0.4 | 2.7 |
| | 4 | | | 4.7 | 0.5 | 2.6 | 0.4 | 2.8 |

### Experiment 5—Construction of M 2543

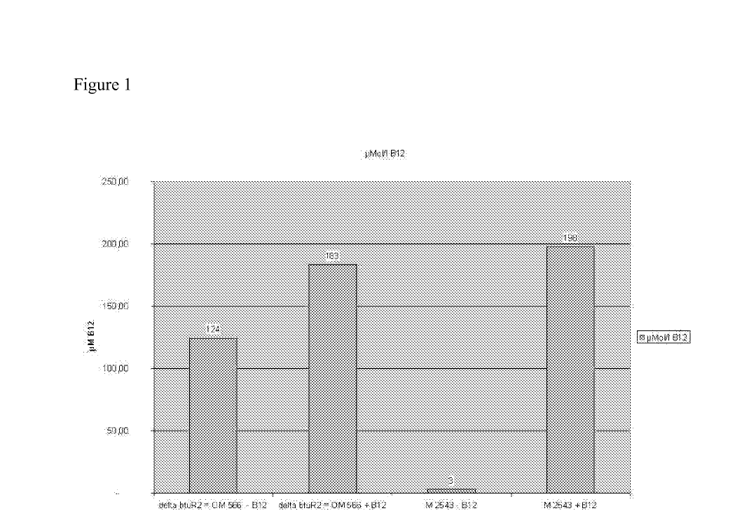

The strain OM469-2 was transformed by electroporation with the plasmid pCLIK5A int sacB PSOD TKT as depicted in SEQ ID NO. 23 (FIG. 1 a)). This was accomplished using the standard Campbelling in and Campbelling out technique.

Isolates of OM 469 PSOD TKT which were labelled M2543 were assayed for methionine production in shake flask culture assays, where they produced more methionine than OM469-2. The results of strain M2543 Are shown in Table 12.

TABLE 12
Shake flask assays of OM469 and M2543

| | | | | | | | | |
|---|---|---|---|---|---|---|---|---|
| | | met | | | | | | |
| | | genes | | | | | | |
| | plas- | on | [Met] | [Lys] | [Gly] | [Hse] | [AHs] | [Ile] |
| Strain | mid | plasmid | (mM) | (mM) | (mM) | (mM) | (mM) | (mM) |

| | | | | | | | |
|---|---|---|---|---|---|---|---|
| OM469-2 | None | 14 | 3.4 | 16 | 1.7 | 0.3 | 11.8 |
| M2543# | None | 20.4 | 1.9 | 21.8 | 0.8 | <0.1 | 12.4 |

### Experiment 6—Construction of OM513, OM589, OM590, OM597 and OM598

The strain OM469 was transformed by electroporation with the replicating plasmid pOM474 (SEQ ID No. 24) which increases expression of the proteins metX, metY and metF. The resulting strain was designated OM513.

Next, two integrating plasmids pOM511 (SEQ ID No. 25) and pOM512 (SEQ ID No. 26) were constructed to enable the construction of C. glutamicum strains with a λP_{L} promoter in place of the btu2 operon promoter. Plasmid pOM511 was used to construct the strain OM589, a derivative OM469 that contains just the λP_{L} promoter replacement. Plasmid pOM512 was used similarly to construct a strain OM590, a derivative of OM469 containing both ΔbtuR2 and the λP_{L} promoter replacements.

The strains were obtained by transforming aforementioned plasmids pOM511 and pOM512 into OM469 by using the standard Campbelling in and out technique.

Subsequently, aforementioned replicating plasmid pOM474 was transformed into OM589 and OM590 to yield OM597 and OM598, respectively.

One approach to assess the effect of modifications assumed to affect btu2 operon expression is to examine the effects of lowered vitamin B12 concentrations on the growth of strains and methionine production in shake flask cultures. Deregulation of vitamin B12 imports should allow strains to more efficiently accumulate vitamin B12 at suboptimal concentration if the suboptimal concentration is greater than the concentration required to repress expression of the native vitamin B12 uptake system. The strains as described above with btu2 operon modifications were assayed at four different vitamin B12 concentrations in shake flask assays. The results as depicted in Table 13 indicate that the strains showed a significant variation in their response to lowered vitamin B12 at a concentration of 100 μg/L. At this vitamin B12 concentration the two strains containing the λP_{L} promoter modification (OM597 and OM598) were able to utilise more glucose and produce more methionine than the two strains lacking this modification.

TABLE 13
Shake flask assay of OM513, OM597 and OM 598

| | | btu2 | | | |
|---|---|---|---|---|---|
| Strain | btuR2 | promoter | [B12] (μg/l) | Glu* | [met] (g/l) |
| OM513 | — | — | 10000 | 0.2 | 5.0 |
| | | | 1000 | 0.3 | 5.3 |
| | | | 100 | 4.8 | 3.6 |
| | | | 10 | 12.1 | 0.7 |
| OM597 | — | λP_{L} | 10000 | 0.3 | 5.1 |
| | | | 1000 | 0.2 | 5.1 |
| | | | 100 | 0.4 | 3.9 |
| | | | 10 | 12.7 | 0.7 |
| OM598 | Δ | λP_{L} | 10000 | 0.4 | 5.0 |
| | | | 1000 | 0.2 | 5.4 |
| | | | 100 | 0.4 | 3.8 |
| | | | 10 | 11.4 | 0.5 |
| * | | | | | |

* remaining glucose (g/l) at end of 48 hours incubation.

Further, two strains, namely OM597 and OM598 were compared in fermentors at vitamin B12 concentrations of 1 mg/l. The strain containing the ΔbtuR2 deletion gave the highest methionine titer of the two strains (23 g/l versus 20 g/l at 36 hours).

### Experiment 7—Construction of OM599

A strain referred to as OM542 was obtained by deleting btuR2 from OM469. To this end, plasmidpOM495 (SEQ ID No. 27) was transformed by electroporation into OM469 and subsequently Campbelled in and Campbelled out to give strain OM542. OM542 was transformed with pOM474 to give strain OM462.

Further, plasmid pOM513 was electroporated and Campbelled in and Campbelled out of OM542 leading to strain OM592, which contained the P_{SOD} promoter driving the tkt gene and downstream genes. Finally, OM592 was transformed with the replicating plasmid, pOM474, to give strain OM599.

Subsequently, strains OM562 and OM599 were compared for their methionine production in shake flasks using the standard molasses medium containing 10 mg/l vitamin B12. The control was OM513, which is OM469 transformed with pOM474. OM562 produced slightly more methionine than OM513, and OM599 produced more than OM562 (see Table 14 below).

TABLE 14
Shake flask assay of OM513, OM562 and OM599
(all containing pOM474)

| | | | tkt | | [Met] |
|---|---|---|---|---|---|
| Strain | | btuR2 | promoter | Glu* | (g/l) |
| OM513-1 | | + | native | 0.3 | 5.2 |
| | | | | 0.3 | 5.6 |
| OM562-1 | | Δ | native | 0.2 | 5.5 |
| OM599- | 1 | Δ | P_{sod} tkt | 0.2 | 6.5 |
| | 2 | | | 0.3 | 6.7 |
| | 3 | | | 0.3 | 6.8 |
| | 4 | | | 0.3 | 6.2 |

*remaining glucose (g/l) at end of 48 hour incubation.

As one can see from Table 14, deletion of btuR2 leads to improved vitamin B12 accumulation.

### Experiment 8—Construction of OM566

A strain referred to as OM566 was obtained by deleting btuR2 from M2543. To this end, plasmidpOM495 (SEQ ID No. 27) was transformed by electroporation into M2543 and subsequently Campbelled in and Campbelled out to give strain OM566.

For the determination of vitamin B12 vitamer uptake, cells were grown in BHI medium with and without the addition of cyanocobalamin 1 mg/l. After overnight growth cells were harvested by centrifugation and washed twice with saline solution (0.85% NaCl). The cells were lyzed using the Ribolyzer procedure as described by the manufacturer Hybaid. Lyzed cells were centrifuged and the supernatent was diluted for the analysis by the vitamin B12 ELISA. The vitamin B12 ELISA Kit from R-Biopharm (Germany) was used according to the description of the manufacturer

Standards and cell lysate samples were diluted in Buffer according to the manufacturer. Samples from C. glutamicum were diluted 1:100 to 1.800 in the ELISA. Values from the linear response curve of the ELISA were taken and the contained B12 vitamer concentration was determined. A protein concentration of 250 mg/ml was taken as a general intracellular protein concentration value for the cytosol of C. glutamicum. From this value and the protein concentration of the lysate the value of sample dilution after cell lysis was determined. Generally the value of error for the B12 determination was found to be approximately 10% from different experiments.

The values of B12 vitamers inside of the cells are reflecting the intracellular hydroxcobalamin concentration, since cyanocobalamin will be converted to hydroxcobalamin.

It was found that the strain M2543 showed a strong increase of hydroxcobalamin from 3 μM to 198 μM in the cell when vitamin B12 was added to the medium at a concentration of 1 mg/l.

The strain OM566 which is M2543 with a deleted btuR2 was found to have a 40 fold increased hydroxcobalamin concentration (124 μM) with no Vitamin B12 added to the BHI Medium. When B12 was added the hydroxcobalamin concentration further increased to 183 μM in OM566 (See also FIG. 1). The result shows that deletion of btuR2 functionally induces the B12 uptake system of. C. glutamicum strains.

The following publications are considered in the context of the invention:
1. Marsh, E N Essays Biochem. 1999; 34: 139-54. Coenzyme B12 (cobalamin)-dependent enzymes.
2. Ludwig M L, Mathhews R G. Annu Rev Biochem. 1997; 66: 269-313. Structure-based perspectives on B12-dependent enzymes.
3. Bandarian V, Matthews R G. Biochemistry. 2001 Apr. 24; 40(16): 5056-64. Quantititation of rate enhancements attained by the binding of cobalamin to methionine synthase.
4. Martens J H, Barg H, Warren M J, Jahn D. Appl. Microbial Biotechnol. 2002 March; 58(3): 275-285. Epub 2001 Dec. 20. Microbial production of vitamin B12.
5. Cadieux N. Bradbeer C, Reeger-Schneider E, Koster W, Mohanty A K, Wiener M C, Kadner R J. J. Bacteriol. 2002 February; 184(3): 706-17.
6. Woodson J D, Reynolds A, Escalante-Semerena J C. J. Bacteriol. 2005 September; 187(17): 5901-9 ABC transporter for corrinoids in Halobacterium sp. strain NRC-1.
7. Johnson C L, Pechonick E, Park S D, Havemann G D, Leal N A, Bobik T A. J. Bacteriol. 2001 March; 183(5): 1577-84. Functional geonomic, biochemical, and genetic characterisation of the Salmonella pduO gene, an ATP:cob(1)alamin adenosyltransferase gene.
8. Johnson C L, Buszko M L, Bobik T A. J. Bacteriol. 2004 December; 186(23): 7881-7 Purification and initial characterisation of the Salmonella enterica PduO ATP:Cob(1)alamin adenosyltransferase.
9. St Maurice M. Mera P E, Taranto M P, Sesma F, Escalante-Semerena J C, Rayment I. J. Biol Chem. 2007 Jan. 26; 282(4): 2596-605. Epub 2006 Nov. 22. Structural characterisation of the active site of the PduO-type ATP: Co(1)rrinoid adenosyl-transferase from Lactobacillus reuteri.

1. A microorganism which is genetically modified to provide increased efficiency of uptake of vitamin B12.

2. The microorganism of claim 1, wherein said increased efficiency is achieved by a deregulated vitamin B12 uptake system.

3. The microorganism of claim 2, wherein said vitamin B12 uptake system comprises nucleic acid sequences encoding at least one negative regulatory protein of SEQ ID No. 2 of functional homologues or fragments thereof and/or at least one ABC-type transporter protein being formed from sub-units of SEQ ID Nos. 4, 6 and 8 or functional homologues or fragments thereof.

4. The microorganism of claim 3, wherein said nucleic acid sequences encoding at least one negative regulatory protein and at least one ABC-type transporter protein are organized as an operon such that said at least one negative regulatory protein modulates expression of said at least one ABC-type transporter protein.

5. The microorganism of claim 3, wherein the amount and/or activity of said at least one negative regulatory protein is at least partially reduced by genetic alteration compared to a respective starting organism not displaying said genetic alteration.

6. The microorganism of claim 3, wherein the amount and/or activity of said at least one ABC-type transporter protein is at least partially increased by genetic alteration compared to a respective starting organism not displaying said genetic alteration.

7. The microorganism of claim 1, wherein the microorganism is selected from gram-positive microorganisms.

8. The microorganism of claim 7, wherein the microorganism is selected from the genus of Corynebacterium.

9. The microorganism of claim 1, wherein the microorganism is a gram-positive microorganism and wherein the vitamin B12 uptake system comprises an operon with nucleic acid sequences encoding at least one negative regulatory protein of SEQ ID No. 2 or functional homologues or fragments thereof and at least one ABC-type transporter protein comprising subunits of SEQ ID Nos. 4, 6 and 8 or functional homologues or fragments thereof.

10. The microorganism of claim 9, wherein the microorganism is selected from the species of C. glutamicum and wherein the vitamin B12 uptake system comprises an operon with nucleic acid sequences encoding at least one negative regulatory protein having at least 50% sequence identity to SEQ ID No. 2 and at least one ABC-type transporter protein comprising subunits having at least 50% sequence identity to SEQ ID Nos. 4, 6 and 8.

11. The microorganism of claim 10, wherein expression of said at least one negative regulatory protein having at least 50% sequence identity to SEQ ID No. 2 is at least partially reduced by genetic alteration and wherein expression of said at least one ABC-type transporter protein comprising subunits having at least 50% sequence identity to SEQ ID Nos. 4, 6 and 8 is at least partially increased by genetic alteration compared to a starting organism not displaying said genetic alterations.

12. The microorganism of claim 11, wherein expression of said at least one negative regulatory protein having at least 50% sequence identity to SEQ ID No. 2 is completely reduced by genetic alteration compared to a starting organism not displaying said genetic alterations.

13. The microorganism of claim 11, wherein expression of said at least one ABC-type transporter protein comprising subunits having at least 50% sequence identity to SEQ ID Nos. 4, 6 and 8 is at least partially increased by a strong promoter and/or by an increased copy number of nucleic acid sequences encoding said subunits compared to a starting organism not displaying said genetic alterations.

14. The microorganism according to claim 1, wherein additionally the amount and/or activity of one or more of the following factors functional homologues and/or functional fragments is increased by genetic alteration compared to a starting organism not displaying said genetic alteration:
metA/X,
metZ/Y,
metF,
metH,
thrA,
metE,
and/or wherein the amount and/or activity of one or more of the following factors functional homologues and/or functional fragments is decreased by genetic alteration compared to a starting organism not displaying said genetic alteration:
metK,
thrB.

15-16. (canceled)

17. A method of obtaining a fine chemical the biological synthesis of which requires vitamin B12 comprising the steps of:
Cultivating a microorganism according to claim 1;
Obtaining said fine chemical.

18. The method of claim 17, wherein said fine chemical is selected from the group comprising methionine, S-adenosyl methionine, and methionine sulfoxide.

19. The microorganism of claim 1, wherein the microorganism is selected from actinobacilli.

20. The microorganism of claim 1, wherein the microorganism is selected from actinomycetes.

21. The microorganism of claim 7, wherein the microorganism is selected from the species of C. glutamicum.
